(19) United States
(12) Patent Application Publication
(10) Pub. No.: US 2025/0059236 A1
(43) Pub. Date: Feb. 20, 2025

(54) BICYCLIC PEPTIDE LIGANDS SPECIFIC FOR CD137

(71) Applicant: BicycleTx Limited, Cambridge (GB)

(72) Inventors: Liuhong Chen, Cambridge (GB); Rachid Lani, Cambridge (GB); Kevin McDonnell, Lexington, MA (US); Gemma Elizabeth Mudd, Cambridge (GB); Peter Park, Lincoln, MA (US)

(21) Appl. No.: 18/742,691

(22) Filed: Jun. 13, 2024

Related U.S. Application Data
(63) Continuation of application No. 17/648,560, filed on Jan. 21, 2022, now Pat. No. 12,049,520.
Application No. 17/648,560 is a division of application No. 16/636,105, filed on Feb. 3, 2020, now Pat. No. 11,261,214.

(30) Foreign Application Priority Data
Aug. 4, 2017 (GB) 1712589.9
Feb. 23, 2018 (GB) 1802934.8
Apr. 9, 2018 (GB) 1805850.3

(51) Int. Cl. C07K 7/64; A61K 38/00; A61K 47/64

(52) U.S. Cl. CPC C07K 7/64; A61K 47/64; A61K 38/00

(57) ABSTRACT

The present invention relates to polypeptides which are covalently bound to molecular scaffolds such that two or more peptide loops are subtended between attachment points to the scaffold. In particular, the invention describes peptides which are high affinity binders of CD137. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by CD137.

### CROSS REFERENCE TO RELATED APPLICATIONS

This application is a continuation application of U.S. application Ser. No. 17/648,560, filed Jan. 21, 2022, which is a divisional application of United States Application No. 16/636, 105, filed Feb. 3, 2020, which is a 371 of International Application No. PCT/GB2018/052222, filed Aug. 3, 2018, which claims the benefit of United Kingdom Application No. GB1805850.3, filed Apr. 9, 2018, United Kingdom Application No. GB1802934.8, filed Feb. 23, 2018, and United Kingdom Application No. GB1712589.9, filed Aug. 4, 2017, the entire contents of each of which are incorporated herein by reference.

### SEQUENCE LISTING

The instant application contains a Sequence Listing which has been submitted electronically in XML file format and is hereby incorporated by reference in its entirety. Said XML copy, created on Oct. 16, 2024, is named 392664-045USD1C1_210462_SL.xml and is 685,876 bytes in size.

### FIELD OF THE INVENTION

The present invention relates to polypeptides which are covalently bound to molecular scaffolds such that two or more peptide loops are subtended between attachment points to the scaffold. In particular, the invention describes peptides which are high affinity binders of CD137. The invention also includes drug conjugates comprising said peptides, conjugated to one or more effector and/or functional groups, to pharmaceutical compositions comprising said peptide ligands and drug conjugates and to the use of said peptide ligands and drug conjugates in preventing, suppressing or treating a disease or disorder mediated by CD137.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7(7), 608-24). Good binding properties result from a relatively large interaction surface formed between the peptide and the target as well as the reduced conformational flexibility of the cyclic structures. Typically, macrocycles bind to surfaces of several hundred-square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favourable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris (bromomethyl) benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a peptide ligand specific for CD137 comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a further aspect of the invention, there is provided a drug conjugate comprising a peptide ligand as defined herein conjugated to one or more effector and/or functional groups.

According to a further aspect of the invention, there is provided a pharmaceutical composition comprising a peptide ligand or a drug conjugate as defined herein in combination with one or more pharmaceutically acceptable excipients.

According to a further aspect of the invention, there is provided a peptide ligand or drug conjugate as defined herein for use in preventing, suppressing or treating a disease or disorder mediated by CD137.

### BRIEF DESCRIPTION OF THE FIGURES

FIG. 1: Results of CD137 cell activity assay using bicyclic peptide BCY592 (SEQ ID NO: 189).

FIG. 2: Competition of CD137L or monoclonal antibody Utomilumab with a fluorescently labelled peptide BCY640 (SEQ ID NO: 49) for binding to CD137 as measured by fluorescence polarization.

FIG. 3: Competition of monoclonal antibodies Utomilumab or Urelumab with a fluorescently labelled peptide BCY640 (SEQ ID NO: 49) for binding to CD137 as measured by fluorescence polarization.

### DETAILED DESCRIPTION OF THE INVENTION

According to one particular aspect of the invention which may be mentioned, there is provided a peptide ligand specific for CD137 comprising a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

In one embodiment, said loop sequences comprise 5 or 6 amino acid acids.

In a further embodiment, said peptide ligand comprises three cysteine residues separated by two loop sequences both of which consist of 6 amino acids.

In a further embodiment, said peptide ligand comprises three cysteine residues separated by two loop sequences one of which consists of 5 amino acids and the other of which consists of 6 amino acids.

In one embodiment, said peptide ligand comprises an amino acid sequence selected from:

(SEQ ID NO: 20)
Cᵢ-I-E-E-G-Q-Y-Cᵢᵢ-X₁-X₂-D-X₃-Y/Q/M-X₄-Cᵢᵢᵢ;
(SEQ ID NO: 21)
Cᵢ-D-I-G-P-P-Y-Cᵢᵢ-Y-R/A-D-M/P-Y-M-Cᵢᵢᵢ;
(SEQ ID NO: 22)
Cᵢ-D-E-W-G-L-F/Y-C-I/F-P/A-H-S/P-D-Cᵢᵢᵢ;
and
(SEQ ID NO: 19)
CᵢIEPGPFCᵢᵢYADPYMCᵢᵢ;

wherein X₁-X₄ represent any amino acid residue and Cᵢ, Cᵢᵢ and Cᵢᵢᵢ represent first, second and third cysteine residues, respectively or a pharmaceutically acceptable salt thereof.

An Alanine scanning experiment was conducted on selected peptides of the invention. An Alanine scan is used to predict which amino acids positions are most amenable to substitutions and further optimisation of affinity and/or other desirable properties. The Alanine scan peptides were characterized into three categories based on affinity relative to the parental peptide sequence (BCY7151; SEQ ID NO: 92): 1. no loss in affinity 2. 2-10 fold weaker affinity and 3. >10 fold weaker affinity. Peptides in category 1 and category 2 can undergo extensive SAR testing with alternative amino acid substitutions. The peptides in category 3 were kept fixed or only substituted with highly similar amino acids. The results of the Alanine scan are shown in Table 2 wherein it can be seen that the Aspartic Acid (D) amino acid residue at position 9 is most important for binding because replacement of this amino acid residue with an Alanine residue eliminated binding activity.

A D-Alanine scanning experiment was also conducted on selected peptides of the invention. The default preparation of all bicyclic peptides is in the L-configuration, therefore, the D-Alanine scan shows which amino acid positions are amenable to D-amino acid substitutions. The results of the D-Alanine scan are shown in Table 2 wherein it can be seen that replacing the position 4 Glycine (G) with D-Ala improved affinity relative to the reference peptide. This implies that the D-Ala4 peptide (BCY7297; SEQ ID NO: 106) is important, since it provides improved affinity as well as other advantages associated with non-natural D isomer amino acids.

In one embodiment, said peptide ligand comprises three cysteine residues separated by two loop sequences both of which consist of 6 amino acids, and said peptide ligand comprises an amino acid sequence selected from:

(SEQ ID NO: 20)
Cᵢ-I-E-E-G-Q-Y-Cᵢᵢ-X₁-X₂-D-X₃-Y/Q/M-X₄-Cᵢᵢᵢ;
(SEQ ID NO: 21)
Cᵢ-D-I-G-P-P-Y-Cᵢᵢ-Y-R/A-D-M/P-Y-M-Cᵢᵢᵢ;
and
(SEQ ID NO: 19)
CᵢIEPGPFCᵢᵢYADPYMCᵢᵢᵢ;

wherein X₁-X₄ represent any amino acid residue and Cᵢ, Cᵢᵢ and Cᵢᵢᵢ represent first, second and third cysteine residues, respectively or a pharmaceutically acceptable salt thereof.

In one embodiment, X₁ is selected from Y, F and H.

In one embodiment, X₂ is selected from R, A and S.

In one embodiment, X₃ is selected from M, P and H.

In one embodiment, X₄ is selected from M, Y, L and F.

In one embodiment, said peptide ligand comprises three cysteine residues separated by two loop sequences the first of which consists of 6 amino acids and the second of which consists of 5 amino acids, and said peptide ligand comprises an amino acid sequence which is:

(SEQ ID NO: 22)
Cᵢ-D-E-W-G-L-F/Y-Cᵢᵢ-I/F-P/A-H-S/P-D-Cᵢᵢᵢ;

wherein Cᵢ, Cᵢᵢ and Cᵢᵢᵢ represent first, second and third cysteine residues, respectively or a pharmaceutically acceptable salt thereof.

In a further embodiment, the peptide ligand of Cᵢ-I-E-E-G-Q-Y-Cᵢᵢ-X₁-X₂-D-X₃-Y/Q/M-X₄-Cᵢᵢᵢ (SEQ ID NO: 20) comprises an amino acid sequence selected from any one of SEQ ID NOS: 1-14:

(SEQ ID NO: 1)
CᵢIEEGQYCᵢᵢYRDMYMCᵢᵢᵢ;
(SEQ ID NO: 2)
CᵢIEEGQYCᵢᵢYADPYMCᵢᵢᵢ;
(SEQ ID NO: 3)
CᵢIEEGQYCᵢᵢYADPYYCᵢᵢᵢ;
(SEQ ID NO: 4)
CᵢIEEGQYCᵢᵢYSDPYYCᵢᵢᵢ;
(SEQ ID NO: 5)
CᵢIEEGQYCᵢᵢFADPYMCᵢᵢᵢ;
(SEQ ID NO: 6)
CᵢIEEGQYCᵢᵢYADHQLCᵢᵢᵢ;
(SEQ ID NO: 7)
CᵢIEEGQYCᵢᵢHADPYYCᵢᵢᵢ;
(SEQ ID NO: 8)
CᵢIEEGQYCᵢᵢHADPYFCᵢᵢᵢ;
(SEQ ID NO: 9)
CᵢIEEGQYCᵢᵢYADHYMCᵢᵢᵢ;
(SEQ ID NO: 10)
CᵢIEEGQYCᵢᵢYADPYLCᵢᵢᵢ;
(SEQ ID NO: 11)
CᵢIEEGQYCᵢᵢYSDPYLCᵢᵢᵢ;
(SEQ ID NO: 12)
CᵢIEEGQYCᵢᵢFADPYLCᵢᵢᵢ;
(SEQ ID NO: 13)
CᵢIEEGQYCᵢᵢHADPYMCᵢᵢᵢ;
and
(SEQ ID NO: 14)
CᵢIEEGQYCᵢᵢHADPQMCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-I-E-E-G-Q-Y-Cᵢᵢ-X₁-X₂-D-X₃-Y/Q/M-X₄-Cᵢᵢᵢ (SEQ ID NO: 20) comprises an amino acid sequence selected from:
A-(SEQ ID NO: 1)-A (herein referred to as 74-01-00-N004);
A-(SEQ ID NO: 2)-A (herein referred to as 74-01-01-N001);
A-(SEQ ID NO: 3)-A (herein referred to as 74-01-02-N001);
A-(SEQ ID NO: 4)-A (herein referred to as 74-01-03-N001);
A-(SEQ ID NO: 5)-A (herein referred to as 74-01-04-N001);
A-(SEQ ID NO: 6)-A (herein referred to as 74-01-05-N001);
A-(SEQ ID NO: 7)-A (herein referred to as 74-01-06-N001);
A-(SEQ ID NO: 8)-A (herein referred to as 74-01-07-N001);
A-(SEQ ID NO: 9)-A (herein referred to as 74-01-08-N001);
A-(SEQ ID NO: 10)-A (herein referred to as 74-01-09-N001);
A-(SEQ ID NO: 10)-SVG (herein referred to as 74-01-09-T03-N002);
A-(SEQ ID NO: 11)-A (herein referred to as 74-01-10-N001);
A-(SEQ ID NO: 12)-A (herein referred to as 74-01-11-N001);
A-(SEQ ID NO: 13)-A (herein referred to as 74-01-13-N001); and
A-(SEQ ID NO: 14)-A (herein referred to as 74-01-14-N001).

In a further embodiment, the peptide ligand of Cᵢ-D-I-G-P-P-Y-Cᵢᵢ-Y-R/A-D-M/P-Y-M-Cᵢᵢᵢ (SEQ ID NO: 21) comprises an amino acid sequence selected from:

(SEQ ID NO: 15)
CᵢDIGPPYCᵢᵢYRDMYMCᵢᵢᵢ;
and
(SEQ ID NO: 16)
CᵢDIGPPYCᵢᵢYADPYMCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-D-I-G-P-P-Y-Cᵢᵢ-Y-R/A-D-M/P-Y-M-Cᵢᵢᵢ (SEQ ID NO: 21) comprises an amino acid sequence selected from:
A-(SEQ ID NO: 15)-A (herein referred to as 74-01-16-N001); and
A-(SEQ ID NO: 16)-A (herein referred to as 74-01-17-N001).

In a further embodiment, the peptide ligand of Cᵢ-D-E-W-G-L-F/Y-Cᵢᵢ-I/F-P/A-H-S/P-D-Cᵢᵢᵢ (SEQ ID NO: 22) comprises an amino acid sequence selected from:

(SEQ ID NO: 17)
CᵢDEWGLFCᵢᵢIPHSDCᵢᵢᵢ;
and
(SEQ ID NO: 18)
CᵢDEWGLYCᵢᵢFAHPDCᵢᵢᵢ;

wherein Cᵢ, Cᵢᵢ and Cᵢᵢᵢ represent first, second and third cysteine residues, respectively, or a pharmaceutically acceptable salt thereof.

In a further embodiment, the peptide ligand of Cᵢ-D-E-W-G-L-F/Y-Cᵢᵢ-I/F-P/A-H-S/P-D-Cᵢᵢᵢ (SEQ ID NO: 22) comprises an amino acid sequence selected from:
Ac-A-(SEQ ID NO: 17)-A (herein referred to as 74-02-00-N004); and
A-(SEQ ID NO: 18)-A (herein referred to as 74-02-01-N001).

In one embodiment, the peptide ligand of CᵢIEPGPFCᵢᵢYADPYMCᵢᵢᵢ (SEQ ID NO: 19) comprises an amino acid sequence of:
A-(SEQ ID NO: 19)-NRV (herein referred to as 74-19-00-T01-N002).

In one embodiment, the molecular scaffold is selected from 1,1′,1″-(1,3,5-triazinane-1,3,5-triyl)triprop-2-en-1-one (TATA) and the peptide ligand comprises an amino acid sequence selected from:
A-(SEQ ID NO: 1)-A (herein referred to as 74-01-00-N004);
A-(SEQ ID NO: 2)-A (herein referred to as 74-01-01-N001);
A-(SEQ ID NO: 3)-A (herein referred to as 74-01-02-N001);
A-(SEQ ID NO: 4)-A (herein referred to as 74-01-03-N001);
A-(SEQ ID NO: 5)-A (herein referred to as 74-01-04-N001);
A-(SEQ ID NO: 6)-A (herein referred to as 74-01-05-N001);
A-(SEQ ID NO: 7)-A (herein referred to as 74-01-06-N001);
A-(SEQ ID NO: 8)-A (herein referred to as 74-01-07-N001);
A-(SEQ ID NO: 9)-A (herein referred to as 74-01-08-N001);
A-(SEQ ID NO: 10)-A (herein referred to as 74-01-09-N001);
A-(SEQ ID NO: 10)-SVG (herein referred to as 74-01-09-T03-N002);
A-(SEQ ID NO: 11)-A (herein referred to as 74-01-10-N001);
A-(SEQ ID NO: 12)-A (herein referred to as 74-01-11-N001);
A-(SEQ ID NO: 13)-A (herein referred to as 74-01-13-N001);
A-(SEQ ID NO: 14)-A (herein referred to as 74-01-14-N001);
A-(SEQ ID NO: 15)-A (herein referred to as 74-01-16-N001);
A-(SEQ ID NO: 16)-A (herein referred to as 74-01-17-N001);
Ac-A-(SEQ ID NO: 17)-A (herein referred to as 74-02-00-N004); and
A-(SEQ ID NO: 18)-A (herein referred to as 74-02-01-N001).

The scaffold/peptide ligands of this embodiment demonstrated superior CD137 competition binding as shown herein in Table 1.

In a yet further embodiment, said peptide ligand is selected from:

(SEQ ID NO: 23)
CᵢIEEGQYCᵢᵢFADPY(Nle)Cᵢᵢᵢ;
(SEQ ID NO: 24)
CᵢIKEGQYCᵢᵢFADPY(Nle)Cᵢᵢᵢ;
(SEQ ID NO: 25)
CᵢIEKGQYCᵢᵢFADPY(Nle)Cᵢᵢᵢ;
(SEQ ID NO: 26)
CᵢIEE(D-K)QYCᵢᵢFADPY(Nle)Cᵢᵢᵢ;
(SEQ ID NO: 27)
CᵢIEEGKYCᵢᵢFADPY(Nle)Cᵢᵢᵢ;
(SEQ ID NO: 28)
CᵢIEEGQYCᵢᵢKADPY(Nle)Cᵢᵢᵢ;
(SEQ ID NO: 29)
CᵢIEEGQYCᵢᵢFADKY(Nle)Cᵢᵢᵢ;
and
(SEQ ID NO: 30)
CᵢIEEGQYCᵢᵢFADPYKCᵢᵢᵢ;

wherein Cᵢ, Cᵢᵢ and Cᵢᵢᵢ represent first, second and third cysteine residues, respectively or a pharmaceutically acceptable salt thereof.

In a yet further embodiment, said peptide ligand comprises N and C terminal modifications and comprises an amino acid sequence selected from:

(SEQ ID NO: 31; BCY3814)
A-CᵢIEEGQYCᵢᵢFADPY(Nle)Cᵢᵢᵢ-A;
(SEQ ID NO: 32; BCY7732)
Ac-A-CᵢIEEGQYCᵢᵢFADPY(Nle)Cᵢᵢᵢ-Dap;
(SEQ ID NO: 33; BCY7733)
Ac-A-CᵢIKEGQYCᵢᵢFADPY(Nle)Cᵢᵢᵢ-A;
(SEQ ID NO: 34; BCY7734)
Ac-A-CᵢIEKGQYCᵢᵢFADPY(Nle)Cᵢᵢᵢ-A;
(SEQ ID NO: 35; BCY7735)
Ac-A-CᵢIEE(D-K)QYCᵢᵢFADPY(Nle)Cᵢᵢᵢ-A;
(SEQ ID NO: 36; BCY7736)
Ac-A-CᵢIEEGKYCᵢᵢFADPY(Nle)Cᵢᵢᵢ-A;
(SEQ ID NO: 37; BCY7737)
Ac-A-CᵢIEEGQYCᵢᵢKADPY(Nle)Cᵢᵢᵢ-A;
(SEQ ID NO: 38; BCY7738)
Ac-A-CᵢIEEGQYCᵢᵢFADKY(Nle)Cᵢᵢᵢ-A;
and
(SEQ ID NO: 39; BCY7739)
Ac-A-CᵢIEEGQYCᵢᵢFADPYKCᵢᵢᵢ-A;

wherein Cᵢ, Cᵢᵢ and Cᵢᵢᵢ represent first, second and third cysteine residues, respectively, Ac represents an N-terminal acetyl group and Dap represents diaminopropionic acid or a pharmaceutically acceptable salt thereof.

In an alternative embodiment, said peptide ligand comprises an amino acid sequence which is:

(SEQ ID NO: 40; 74-22-00)
CᵢLPPGQYCᵢᵢFPDLLLCᵢᵢᵢ

wherein Cᵢ, Cᵢᵢ and Cᵢᵢᵢ represent first, second and third cysteine residues, respectively.

In an alternative embodiment, said peptide ligand comprises three cysteine residues separated by two loop sequences both of which consist of 6 amino acids, and said peptide ligand comprises an amino acid sequence which is:

(SEQ ID NO: 41)
Cᵢ-I/L/M/V-E/D/P/S-P/E/A-G-P/Q-Y/F-Cᵢᵢ-Y-A-D-P-
Y/M-M/L/Y-Cᵢᵢᵢ;

wherein Cᵢ, Cᵢᵢ and Cᵢᵢᵢ represent first, second and third cysteine residues, respectively.

In a further embodiment, said amino acid sequence is selected from the peptide sequences listed in Table 1. In a yet further embodiment, said amino acid sequence is selected from the peptide sequences listed in Table 1 excluding the peptides of BCY7238 (SEQ ID NO: 51), BCY7241 (SEQ ID NO: 54), BCY7243 (SEQ ID NO: 56) and BCY7246 (SEQ ID NO: 59). The peptides of this embodiment were tested in the CD137 direct binding assay and demonstrated good levels of binding.

In a further embodiment, said amino acid sequence is selected from the peptide sequences listed in Table 3. The peptides of this embodiment were tested in the CD137 direct binding assay and demonstrated good levels of binding.

In a further embodiment, said amino acid sequence is selected from the peptide sequences listed in Tables 4 and 5. The peptides of this embodiment were tested in the CD137 SPR assay and demonstrated good levels of binding.

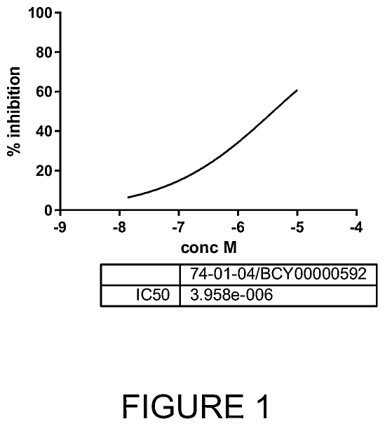

In a further embodiment, said amino acid sequence is selected from BCY592 (SEQ ID NO: 189). Data is presented herein in FIG. 1 which shows that the bicyclic peptide BCY592 (SEQ ID NO: 189) inhibited CD137L activity in a cell-based assay.

In an alternative embodiment, said peptide ligand comprises three cysteine residues separated by two loop sequences both of which consist of 6 amino acids, and said peptide ligand comprises an amino acid sequence which is:

(SEQ ID NO: 266)
Cᵢ-X₅-X₆-X₇-X₈-X₉-X₁₀-Cᵢᵢ-X₁₁-X₁₂-D-X₁₃-X₁₄-X₁₅-Cᵢᵢᵢ;

wherein Cᵢ, Cᵢᵢ and Cᵢᵢᵢ represent first, second and third cysteine residues, respectively;
X₅ represents Ile, tBuAla or Chg;
X₆ represents Glu, Pro, Asp, Lys, Aad, HyP or Oxa;
X₇ represents Glu, Lys or Aad;
X₈ represents Gly, D-Lys, D-Ala, L-Ala, D-Phe, D-Glu, D-Gln, D-Leu, D-Ser or D-Trp;
X₉ represents Gln, Lys, Ala, Pro, 5,5-dmP, Oic, Oxa, HyP, Aib or Ac5c;
X₁₀ represents Tyr, Phe, 3MePhe, 4MePhe, 4FPhe, 2Nal, 4MeOPhe or 4,4-BPA;
X₁₁ represents Phe, Lys, 4MePhe, 2FPhe, 4FPhe, 4Pal, 4,4-BPA, 4tBuPhe, NO2Phe or 4BrPhe;
X₁₂ represents Ala or Lys;
X₁₃ represents Pro or Lys;
X₁₄ represents Tyr or Lys; and
X₁₅ represents Met, Lys, Nle, HLeu or Ahp.

In one embodiment, the peptide ligand of SEQ ID NO: 266 is selected from the C, to Cil sequences of the following peptides (i.e. absent any N-terminal and C-terminal additions): BCY7239 (SEQ ID NO: 52), BCY7240 (SEQ ID NO: 53), BCY7242 (SEQ ID NO: 55), BCY7244 (SEQ ID NO: 57), BCY7245 (SEQ ID NO: 58), BCY7247 (SEQ ID NO: 60), BCY7248 (SEQ ID NO: 61), BCY7249 (SEQ ID NO: 62), BCY7416 (SEQ ID NO: 63), BCY7287 (SEQ ID NO: 97), BCY7297 (SEQ ID NO: 106), BCY7154 (SEQ ID NO: 117), BCY7156 (SEQ ID NO: 119), BCY7157 (SEQ ID NO: 120), BCY7158 (SEQ ID NO: 121), BCY7162 (SEQ ID NO: 125), BCY7165 (SEQ ID NO: 128), BCY7166 (SEQ ID NO: 129), BCY7167 (SEQ ID NO: 130), BCY7168 (SEQ ID NO: 131), BCY7169 (SEQ ID NO: 132), BCY7170 (SEQ ID NO: 133), BCY7174 (SEQ ID NO: 136), BCY7175 (SEQ ID NO: 137), BCY7177 (SEQ ID NO: 139), BCY7178 (SEQ ID NO: 140), BCY7179 (SEQ ID NO: 141), BCY7183 (SEQ ID NO: 145), BCY7185 (SEQ ID NO: 147), BCY7195 (SEQ ID NO: 156), BCY7198 (SEQ ID NO: 159), BCY7211 (SEQ ID NO: 170), BCY7311 (SEQ ID NO: 188), BCY7768 (SEQ ID NO: 195), BCY7770 (SEQ ID NO: 196), BCY7772 (SEQ ID NO: 197), BCY7773 (SEQ ID NO: 198), BCY7774 (SEQ ID NO: 199), BCY7775 (SEQ ID NO: 200), BCY7776 (SEQ ID NO: 201), BCY7796 (SEQ ID NO: 203), BCY7798 (SEQ ID NO: 204), BCY7801 (SEQ ID NO: 207), BCY7802 (SEQ ID NO: 208), BCY7936 (SEQ ID NO: 223), BCY7941 (SEQ ID NO: 227), BCY7942 (SEQ ID NO: 228), BCY7944 (SEQ ID NO: 230), BCY7950 (SEQ ID NO: 232), BCY7954 (SEQ ID NO: 234), BCY7958 (SEQ ID NO: 238), BCY7959 (SEQ ID NO: 239), BCY7960 (SEQ ID NO: 240), BCY7952 (SEQ ID NO: 241), BCY7961 (SEQ ID NO: 242), BCY8656 (SEQ ID NO: 245), BCY8659 (SEQ ID NO: 248), BCY8663 (SEQ ID NO: 252), BCY8668 (SEQ ID NO: 256), BCY8669 (SEQ ID NO: 257), BCY8674 (SEQ ID NO: 261), BCY8675 (SEQ ID NO: 262), BCY9273 (SEQ ID NO: 265), BCY3814 (SEQ ID NO: 31), BCY7527 (SEQ ID NO: 194), and BCY7965 (SEQ ID NO: 243).

In a further embodiment, the peptide ligand of SEQ ID NO: 266 is selected from the full sequences of the following peptides (i.e. including all N-terminal and C-terminal additions): BCY7239 (SEQ ID NO: 52), BCY7240 (SEQ ID NO: 53), BCY7242 (SEQ ID NO: 55), BCY7244 (SEQ ID NO: 57), BCY7245 (SEQ ID NO: 58), BCY7247 (SEQ ID NO: 60), BCY7248 (SEQ ID NO: 61), BCY7249 (SEQ ID NO: 62), BCY7416 (SEQ ID NO: 63), BCY7287 (SEQ ID NO: 97), BCY7297 (SEQ ID NO: 106), BCY7154 (SEQ ID NO: 117), BCY7156 (SEQ ID NO: 119), BCY7157 (SEQ ID NO: 120), BCY7158 (SEQ ID NO: 121), BCY7162 (SEQ ID NO: 125), BCY7165 (SEQ ID NO: 128), BCY7166 (SEQ ID NO: 129), BCY7167 (SEQ ID NO: 130), BCY7168 (SEQ ID NO: 131), BCY7169 (SEQ ID NO: 132), BCY7170 (SEQ ID NO: 133), BCY7174 (SEQ ID NO: 136), BCY7175 (SEQ ID NO: 137), BCY7177 (SEQ ID NO: 139), BCY7178 (SEQ ID NO: 140), BCY7179 (SEQ ID NO: 141), BCY7183 (SEQ ID NO: 145), BCY7185 (SEQ ID NO: 147), BCY7195 (SEQ ID NO: 156), BCY7198 (SEQ ID NO: 159), BCY7211 (SEQ ID NO: 170), BCY7311 (SEQ ID NO: 188), BCY7768 (SEQ ID NO: 195), BCY7770 (SEQ ID NO: 196), BCY7772 (SEQ ID NO: 197), BCY7773 (SEQ ID NO: 198), BCY7774 (SEQ ID NO: 199), BCY7775 (SEQ ID NO: 200), BCY7776 (SEQ ID NO: 201), BCY7796 (SEQ ID NO: 203), BCY7798 (SEQ ID NO: 204), BCY7801 (SEQ ID NO: 207), BCY7802 (SEQ ID NO: 208), BCY7936 (SEQ ID NO: 223), BCY7941 (SEQ ID NO: 227), BCY7942 (SEQ ID NO: 228), BCY7944 (SEQ ID NO: 230), BCY7950 (SEQ ID NO: 232), BCY7954 (SEQ ID NO: 234), BCY7958 (SEQ ID NO: 238), BCY7959 (SEQ ID NO: 239), BCY7960 (SEQ ID NO: 240), BCY7952 (SEQ ID NO: 241), BCY7961 (SEQ ID NO: 242), BCY8656 (SEQ ID NO: 245), BCY8659 (SEQ ID NO: 248), BCY8663 (SEQ ID NO: 252), BCY8668 (SEQ ID NO: 256), BCY8669 (SEQ ID NO: 257), BCY8674 (SEQ ID NO: 261), BCY8675 (SEQ ID NO: 262), BCY9273 (SEQ ID NO: 265), BCY3814 (SEQ ID NO: 31), BCY7527 (SEQ ID NO: 194), and BCY7965 (SEQ ID NO: 243).

These peptides either all demonstrated good levels of binding in the direct binding or SPR assays described herein or represented peptides which are tolerant of substitution with a Lys residue.

In one embodiment, X₅ represents Ile or tBuAla.

In one embodiment, X₆ represents Lys, Glu or Pro.

In one embodiment, X₇ represents Glu or D-Lys.

In one embodiment, X₈ represents Gly, D-Lys, D-Phe or D-Ala.

In one embodiment, X₉ represents Gln, Lys or Pro.

In one embodiment, X₁₀ represents Tyr or 4MePhe.

In one embodiment, X₁₁ represents Phe or 4FPhe.

In one embodiment, X₁₂ represents Ala.

In one embodiment, X₁₃ represents Pro.

In one embodiment, X₁₄ represents Tyr.

In one embodiment, X₁₅ represents Met or Nle.

In a further embodiment, said peptide ligand comprises three cysteine residues separated by two loop sequences both of which consist of 6 amino acids, and said peptide ligand comprises an amino acid sequence which is:

(SEQ ID NO: 267)
Cᵢ-X₅-X₆-X₇-X₈-X₉-X₁₀-Cᵢᵢ-X₁₁-A-D-P-Y-X₁₅-Cᵢᵢᵢ;

wherein Cᵢ, Cᵢᵢ and Cᵢᵢᵢ represent first, second and third cysteine residues, respectively;
X₅ represents Ile or tBuAla;
X₆ represents Lys, Glu or Pro;
X₇ represents Glu or D-Lys;
X₈ represents Gly, D-Lys, D-Phe or D-Ala;
X₉ represents Gln, Lys or Pro;
X₁₀ represents Tyr or 4MePhe;
X₁₁ represents Phe or 4FPhe; and
X₁₅ represents Met or Nle.

In one embodiment, the peptide ligand of SEQ ID NO: 267 is selected from the Cᵢ to Cᵢᵢᵢ sequences of the following peptides (i.e. absent any N-terminal and C-terminal additions): BCY7239 (SEQ ID NO: 52), BCY7240 (SEQ ID NO: 53), BCY7242 (SEQ ID NO: 55), BCY7416 (SEQ ID NO: 63), BCY7156 (SEQ ID NO: 119), BCY7166 (SEQ ID NO: 129), BCY7174 (SEQ ID NO: 136), BCY7774 (SEQ ID NO: 199), BCY9273 (SEQ ID NO: 265), BCY3814 (SEQ ID NO: 31), BCY7527 (SEQ ID NO: 194), and BCY7965 (SEQ ID NO: 243).

In a further embodiment, the peptide ligand of SEQ ID NO: 267 is selected from the full sequences of the following peptides (i.e. including all N-terminal and C-terminal additions): BCY7239 (SEQ ID NO: 52), BCY7240 (SEQ ID NO: 53), BCY7242 (SEQ ID NO: 55), BCY7416 (SEQ ID NO: 63), BCY7156 (SEQ ID NO: 119), BCY7166 (SEQ ID NO: 129), BCY7174 (SEQ ID NO: 136), BCY7774 (SEQ ID NO: 199), BCY9273 (SEQ ID NO: 265), BCY3814 (SEQ ID NO: 31), BCY7527 (SEQ ID NO: 194), and BCY7965 (SEQ ID NO: 243).

These peptides either all demonstrated excellent levels of binding in the direct binding or SPR assays described herein or represented peptides which are tolerant of substitution with a Lys residue.

In one embodiment, the peptide ligand of SEQ ID NO: 267 is selected from the Cᵢ to Cᵢᵢᵢsequences of the following peptides (i.e. absent any N-terminal and C-terminal additions):

BCY7239 (SEQ ID NO: 52), BCY7240 (SEQ ID NO: 53), BCY7242 (SEQ ID NO: 55), BCY7416 (SEQ ID NO: 63).

In a further embodiment, the peptide ligand of SEQ ID NO: 267 is selected from the full sequences of the following peptides (i.e. including all N-terminal and C-terminal additions): BCY7239 (SEQ ID NO: 52), BCY7240 (SEQ ID NO: 53), BCY7242 (SEQ ID NO: 55), BCY7416 (SEQ ID NO: 63).

These peptides represented peptides which are tolerant of substitution with a Lys residue.

In one embodiment, the peptide ligand of SEQ ID NO: 267 is selected from the Cᵢ to Cᵢᵢᵢ sequences of the following peptides (i.e. absent any N-terminal and C-terminal additions): BCY9273 (SEQ ID NO: 265), BCY3814 (SEQ ID NO: 31), BCY7527 (SEQ ID NO: 194), and BCY7965 (SEQ ID NO: 243).

In a further embodiment, the peptide ligand of SEQ ID NO: 267 is selected from the full sequences of the following peptides (i.e. including all N-terminal and C-terminal additions): BCY9273 (SEQ ID NO: 265), BCY3814 (SEQ ID NO: 31), BCY7527 (SEQ ID NO: 194), and BCY7965 (SEQ ID NO: 243).

These peptides either all demonstrated good levels of binding in the direct binding or SPR assays described herein.

In one embodiment, the peptide ligand of SEQ ID NO: 267 is selected from the Cᵢ to Cᵢᵢᵢ sequences of the following peptides (i.e. absent any N-terminal and C-terminal additions): BCY7156 (SEQ ID NO: 119), BCY7166 (SEQ ID NO: 129), BCY7174 (SEQ ID NO: 136), BCY7774 (SEQ ID NO: 199).

In a further embodiment, the peptide ligand of SEQ ID NO: 267 is selected from the full sequences of the following peptides (i.e. including all N-terminal and C-terminal additions): BCY7156 (SEQ ID NO: 119), BCY7166 (SEQ ID NO: 129), BCY7174 (SEQ ID NO: 136), BCY7774 (SEQ ID NO: 199).

These peptides either all demonstrated excellent levels of binding in the direct binding or SPR assays described herein.

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3^{rd} ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within compounds of formula (I), cysteine residues (Cᵢ, Cᵢᵢ and Cᵢᵢᵢ ) are omitted from the numbering as they are invariant, therefore, the numbering of amino acid residues within the compound of formula (I) is referred to as below:

(SEQ ID NO: 1)
-Cᵢ-I₁-E₂-E₃-G₄-Q₅-Y₆-Cᵢᵢ-Y₇-R₈-D₉-M₁₀-Y₁₁-M₁₂-Cᵢᵢᵢ-

For the purpose of this description, all bicyclic peptides are assumed to be cyclised with TBMB (1,3,5-tris(bromomethyl)benzene) or 1,1′,1″-(1,3,5-triazinane-1,3,5-triyl)triprop-2-en-1-one (TATA) and yielding a tri-substituted structure. Cyclisation with TBMB and TATA occurs on Cᵢ, Cᵢᵢ and Cᵢᵢᵢ.

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A-

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ ), and form at least two loops on the scaffold.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement preclinical pharmacodynamics and pharmacokinetic evaluation;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes; and
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent.
Selectivity. Certain peptide ligands of the invention demonstrate good selectivity over other CDs.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of formula (I) contain an amine function, these may form quaternary ammonium salts, for example by reaction with an alkylating agent according to methods well known to the skilled person. Such quaternary ammonium compounds are within the scope of formula (I).

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

### Isotopic variations

The present invention includes all pharmaceutically acceptable (radio)isotope-labelled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio) isotopes, and peptide ligands of the invention, wherein certain functional groups are covalently replaced with relevant (radio) isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulphur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the CD137 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labelled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of hexahydro-1,3,5-triazine, especially 1,3,5-triacryloylhexahydro-1,3,5-triazine (‘TATA’), or a derivative thereof.

In one embodiment, the molecular scaffold is 2,4,6-tris(bromomethyl)mesitylene. This molecule is similar to 1,3,5-tris(bromomethyl)benzene (TBMB) but contains three additional methyl groups attached to the benzene ring. This has the advantage that the additional methyl groups may form further contacts with the polypeptide and hence add additional structural constraint.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene or iodoacetamide. Other scaffold reactive groups that are used to selectively couple compounds to cysteines in proteins are maleimides, αβ unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an αβ unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606). An example of an α-halomethylcarbonyl containing compound is N,N′,N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

### Reactive Groups

The molecular scaffold of the invention may be bonded (i.e. covalently) to the polypeptide via functional or reactive groups on the polypeptide. These are typically formed from the side chains of particular amino acids found in the polypeptide polymer. Such reactive groups may be a cysteine side chain, a lysine side chain, or an N-terminal amine group or any other suitable reactive group. Details may be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

In one embodiment, the reactive group comprises a cysteine residue. In an alternative embodiment, the reactive group comprises penicillamine.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment of the invention, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved. In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5(7), 502-7.

### Effector and Functional Groups

According to a further aspect of the invention, there is provided a drug conjugate comprising a peptide ligand as defined herein conjugated to one or more effector and/or functional groups.

Effector and/or functional groups can be attached, for example, to the N and/or C termini of the polypeptide, to an amino acid within the polypeptide, or to the molecular scaffold.

Appropriate effector groups include antibodies and parts or fragments thereof. For instance, an effector group can include an antibody light chain constant region (CL), an antibody CH1 heavy chain domain, an antibody CH2 heavy chain domain, an antibody CH3 heavy chain domain, or any combination thereof, in addition to the one or more constant region domains. An effector group may also comprise a hinge region of an antibody (such a region normally being found between the CH1 and CH2 domains of an IgG molecule).

In a further embodiment of this aspect of the invention, an effector group according to the present invention is an Fc region of an IgG molecule. Advantageously, a peptide ligand-effector group according to the present invention comprises or consists of a peptide ligand Fc fusion having a tβ (half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p821; Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi et al (1994) J Biol. Chem. Volume 269 p10444), the 18 amino acid ‘model amphipathic peptide’ (Oehlke et al (1998) Biochim Biophys Acts Volume 1414 p127) and arginine rich regions of the HIV TAT protein. Non peptidic approaches include the use of small molecule mimics or SMOCs that can be easily attached to biomolecules (Okuyama et al (2007) Nature Methods Volume 4 p153). Other chemical strategies to add guanidinium groups to molecules also enhance cell penetration (Elson-Scwab et al (2007) J Biol Chem Volume 282 p13585). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, 15 days or more or 20 days or more. Advantageously a peptide ligand-effector group or composition according to the invention will have a tβ half-life in the range 12 to 60 hours. In a further embodiment, it will have a tβ half-life of a day or more. In a further embodiment still, it will be in the range 12 to 26 hours.

In one particular embodiment of the invention, the functional group is selected from a metal chelator, which is suitable for complexing metal radioisotopes of medicinal relevance.

Possible effector groups also include enzymes, for instance such as carboxypeptidase G2 for use in enzyme/prodrug therapy, where the peptide ligand replaces antibodies in ADEPT.

In one particular embodiment of the invention, the functional group is selected from a drug, such as a cytotoxic agent for cancer therapy. Suitable examples include: alkylating agents such as cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol;

topoisomerase inhibitors including camptothecins: irinotecan and topotecan, and type II inhibitors including amsacrine, etoposide, etoposide phosphate, and teniposide. Further agents can include antitumour antibiotics which include the immunosuppressant dactinomycin (which is used in kidney transplantations), doxorubicin, epirubicin, bleomycin, calicheamycins, and others.

In one further particular embodiment of the invention, the cytotoxic agent is selected from maytansinoids (such as DM1) or monomethyl auristatins (such as MMAE).

DM1 is a cytotoxic agent which is a thiol-containing derivative of maytansine and has the following structure:

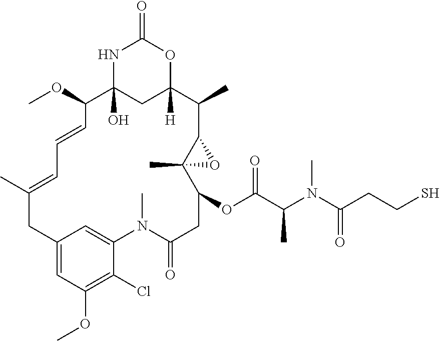

Monomethyl auristatin E (MMAE) is a synthetic antineoplastic agent and has the following structure:

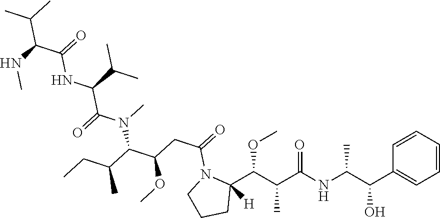 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In one yet further particular embodiment of the invention, the cytotoxic agent is selected from maytansinoids (such as DM1).

In one embodiment, the cytotoxic agent is linked to the bicyclic peptide by a cleavable bond, such as a disulphide bond or a protease sensitive bond. In a further embodiment, the groups adjacent to the disulphide bond are modified to control the hindrance of the disulphide bond, and by this the rate of cleavage and concomitant release of cytotoxic agent.

Published work established the potential for modifying the susceptibility of the disulphide bond to reduction by introducing steric hindrance on either side of the disulphide bond (Kellogg et al (2011) Bioconjugate Chemistry, 22, 717). A greater degree of steric hindrance reduces the rate of reduction by intracellular glutathione and also extracellular (systemic) reducing agents, consequentially reducing the ease by which toxin is released, both inside and outside the cell. Thus, selection of the optimum in disulphide stability in the circulation (which minimises undesirable side effects of the toxin) versus efficient release in the intracellular milieu (which maximises the therapeutic effect) can be achieved by careful selection of the degree of hindrance on either side of the disulphide bond.

The hindrance on either side of the disulphide bond is modulated through introducing one or more methyl groups on either the targeting entity (here, the bicyclic peptide) or toxin side of the molecular construct.

In one embodiment, the cytotoxic agent and linker is selected from any combinations of those described in WO 2016/067035 (the cytotoxic agents and linkers thereof are herein incorporated by reference).

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio) conjugation techniques may be used to introduce an activated or activatable N-or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al Proc Natl Acad Sci U S A. 1994 Dec. 20; 91(26): 12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulphide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand or a drug conjugate as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16^{th} Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as CD137 binding agents.

CD137 is a member of the tumour necrosis factor (TNF) receptor family. Its alternative names are tumour necrosis factor receptor superfamily member 9 (TNFRSF9), 4-IBB and induced by lymphocyte activation (ILA). CD137 can be expressed by activated T cells, but to a larger extent on CD8+ than on CD4+ T cells. In addition, CD137 expression is found on dendritic cells, follicular dendritic cells, natural killer cells, granulocytes and cells of blood vessel walls at sites of inflammation. One characterized activity of CD137 is its costimulatory activity for activated T cells. Crosslinking of CD137 enhances T cell proliferation, IL-2 secretion, survival and cytolytic activity. Further, it can enhance immune activity to eliminate tumours in mice.

CD137 is a T-cell costimulatory receptor induced on TCR activation (Nam et al., Curr. Cancer Drug Targets, 5:357-363 (2005); Waits et al., Annu. Rev, Immunol., 23:23-68 (2005)). In addition to its expression on activated CD4+ and CD8+ T cells, CD137 is also expressed on CD4+CD25+ regulatory T cells, natural killer (NK) and NK-T cells, monocytes, neutrophils, and dendritic cells. Its natural ligand, CD137L, has been described on antigen-presenting cells including B cells, monocyte/macrophages, and dendritic cells (Watts et al. Annu. Rev. Immunol, 23:23-68 (2005)). On interaction with its ligand, CD137 leads to increased TCR-induced T-cell proliferation, cytokine production, functional maturation, and prolonged CD8+ T-cell survival (Nam et al, Curr. Cancer Drug Targets, 5:357-363 (2005), Watts et al., Annu. Rev. Immunol, 23:23-68 (2005)).

Signalling through CD137 by either CD137L or agonistic monoclonal antibodies (mAbs) against CD137 leads to increased TCR-induced T cell proliferation, cytokine production and functional maturation, and prolonged CD8+ T cell survival. These effects result from: (1) the activation of the NF-KB, c-Jun NH2-terminal kinase/stress-activated protein kinase (JNK/SAPK), and p38 mitogen-activated protein kinase (MAPK) signalling pathways, and (2) the control of anti-apoptotic and cell cycle-related gene expression.

Experiments performed in both CD137 and CD137L-deficient mice have additionally demonstrated the importance of CD137 costimulation in the generation of a fully competent T cell response.

IL-2 and IL-15 activated NK cells express CD137, and ligation of CD137 by agonistic mAbs stimulates NK cell proliferation and IFN-γ secretion, but not their cytolytic activity.

Furthermore, CD137-stimulated NK cells promote the expansion of activated T cells in vitro.

In accordance with their costimulatory function, agonist mAbs against CD137 have been shown to promote rejection of cardiac and skin allografts, eradicate established tumours, broaden primary antiviral CD8+ T cell responses, and increase T cell cytolytic potential. These studies support the view that CD137 signalling promotes T cell function which may enhance immunity against tumours and infection.

Polypeptide ligands selected according to the method of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand or a drug conjugate as defined herein, for use in preventing, suppressing or treating a disease or disorder mediated by CD137.

According to a further aspect of the invention, there is provided a method of preventing, suppressing or treating a disease or disorder mediated by CD137, which comprises administering to a patient in need thereof an effector group and drug conjugate of the peptide ligand as defined herein.

In one embodiment, the CD137 is mammalian CD137. In a further embodiment, the mammalian CD137 is human CD137 (hCD137).

In one embodiment, the disease or disorder mediated by CD137 is selected from cancer, infection and inflammation. In a further embodiment, the disorder or disease mediated by CD137 is selected from cancer.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the oesophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukaemias, lymphomas) and premalignant haematological disorders and disorders of borderline malignancy including haematological malignancies and related conditions of lymphoid lineage (for example acute lymphocytic leukaemia [ALL], chronic lymphocytic leukaemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukaemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, sarcoma, synovial sarcomas, epithelioid sarcomas, Kaposi's sarcoma, Ewing's gastrointestinal stromal tumours, benign and malignant histiocytomas, and dermatofibrosarcomaprotuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukaemia (B-CLL), B and T acute lymphocytic leukaemia (ALL), T cell lymphoma (TCL), acute myeloid leukaemia (AML), hairy cell leukaemia (HCL), Hodgkin's Lymphoma (HL), and chronic myeloid leukaemia (CML).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology. Peptides were purified using HPLC and following isolation they were modified with 1,3,5-triacryloylhexahydro-1,3,5-triazine (TATA, Sigma). For this, linear peptide was diluted with 50:50 MeCN:H₂O up to ˜35 mL, ˜500 μL of 100 mM TATA in acetonitrile was added, and the reaction was initiated with 5 mL of 1 M NH₄HCO₃ in H₂O. The reaction was allowed to proceed for ˜30-60 min at RT, and lyophilised once the reaction had completed (judged by MALDI). Once completed, 1 ml of 1 M L-cysteine hydrochloride monohydrate (Sigma) in H₂O was added to the reaction for ˜60 min at RT to quench any excess TATA.

Following lyophilisation, the modified peptide was purified as above, while replacing the Luna C8 with a Gemini C18 column (Phenomenex), and changing the acid to 0.1% trifluoroacetic acid. Pure fractions containing the correct TATA-modified material were pooled, lyophilised and kept at −20° C. for storage.

All amino acids, unless noted otherwise, were used in the L-configurations.

In some cases, peptides are converted to activated disulphides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Abbreviations

Aad 2-Aminoadipic acid
Abu 2-Aminobutyric acid
Ac5c Aminocyclopentanecarboxylic acid
Ahp Aminoheptanoic acid
Aib aminoisobutyric acid
Me-Ala methyl alanine
NMe-Ala N-methyl alanine
tBuAla t-butyl-Alanine
Api Amino pimelic acid
Aze Azetidine
4,4-BPA 4,4-Biphenylalanine
CF3G Trifluoromethyl-Alanine
Cha 3-cyclohexyl alanine
Chg L-Cyclohexyl glycine
Cit citrulline
H-Cys homocysteine
Dap diaminopimelic acid
FI fluorescein
NMeGlu N-methyl glutamic acid
HGIn homoglutamine
HyP hydroxyproline
Hleu homoleucine
Nle norleucine
Na naphthylalanine
NMelle N-Methyl-Isoleucine
Oic octahydroindolecarboxylic acid
Oxa oxazolidine-4-carboxylic acid
Pal pyridylalanine
Pen penicillamine
pCaPhe para-Carbamoyl-Phenylalanine
pCoPhe para-Carboxy-Phenylalanine
Phg phenylglycine
HPhe homophenylalanine
FPhe fluorophenylalanine
MePhe methyl phenylalanine
MeOPhe methoxy phenylalanine
tBuPhe t-butyl phenylalanine
NO2Phe nitro phenylalanine
BrPhe bromo phenylalanine
Pip Pipecolic acid
5,5-dmP 5,5-Dimethyl-L-Proline
Sar sarcosine
HSe (me) Homoserine (Me)
TetraZ tetrazole alanine
NMeTyr N-methyl tyrosine

### BIOLOGICAL DATA

### 1. CD137 Direct Binding Assay

Affinity of the peptides of the invention for human CD137 (Ki) was determined using a fluorescence polarisation assay, in accordance with the methods disclosed in WO 2016/067035. Peptides of the invention were labelled with a fluorescent tag (fluorescein, Fl) and diluted to 2.5 nM in 50 mM HEPES with 100 mM NaCl and 0.05% tween pH 7.5. CD137protein was titrated starting at 3 μM in the same assay buffer as the peptide to assay 1 nM peptide in a total volume of 25 μL in black walled and bottomed low bind low volume 384 well plates. The assay was typically set up by adding 5 μL assay buffer, 10 μL CD137 protein then 10 μL fluorescent peptide. The concentrations of CD137 protein were 1 in 2 serial dilutions to give 12 different concentrations starting at 3 μM. Measurements were conducted on a BMG PHERAstar FS equipped with an FP 485 520 520 optic module at 25° C. with 200 flashes per well and a positioning delay of 0.1 second. Alternatively, the measurements were performed using Envision (PerkinElmer) equipped with FITC FP Dual Enh mirror, set to 30 flashes. Each well was measured every 5 minutes for 60 minutes. The gain used for analysis was determined for each tracer at the end of the 60 minutes where there was no protein in the well. The mP were fit to a standard 1:1 binding model with a quadratic equation to generate a Kd value. Selected peptides of the invention were tested in the above mentioned assay and the results are shown in Tables 1 to 3:

TABLE 1
Direct Binding Results with Selected Peptides

| Peptide | SEQ ID | | | |
|---|---|---|---|---|
| Number | NO: | Peptide Reference | Sequence | Kd nM |
| BCY633 | 42 | (B-Ala)-Sar5-(74-01-00) | [B-Ala]-Sar5-ACIEEGQYCYRDMYMCA | 1841 |
| BCY634 | 43 | Ac-(74-01-00)-Sar6-K(FI) | [Ac]ACIEEGQYCYRDMYMCA-Sar6-K(FI) | 1376 |
| BCY636 | 44 | (74-01-01)-Sar6-K(FI) | ACIEEGQYCYADPYMCA-Sar6-K(FI) | 123.9 |
| BCY635 | 45 | (B-Ala)-Sar5-(74-01-01) | [B-Ala]-Sar5-ACIEEGQYCYADPYMCA | 126 |
| BCY638 | 46 | (74-01-02)-Sar6-K | ACIEEGQYCYADPYYCASar6-K | 192.5 |
| BCY637 | 47 | (B-Ala)-Sar5-(74-01-02) | [B-Ala]-Sar5-ACIEEGQYCYADPYYCA | 122 |
| BCY639 | 48 | (74-01-03)-Sar6-K | ACIEEGQYCYSDPYYCA-Sar6-K | 229 |
| BCY640 | 49 | (74-01-04)-Sar6-K | ACIEEGQYCFADPYMCA-Sar6-K | 84 |
| BCY641 | 50 | G-Sar5-(74-01-04) | G-Sar5-ACIEEGQYCFADPYMCA | 152.5 |
| BCY7238 | 51 | Ac-(74-01-04)Lys1(PEG12) | Ac-CK(Peg12)EEGQYCFADPYMC | >>3000 |
| BCY7239 | 52 | Ac-(74-01-04)Lys2(PEG12) | Ac-CIK(Peg12)EGQYCFADPYMC | 579 |
| BCY7240 | 53 | Ac-(74-01-04)Lys3(PEG12) | Ac-CIEK(Peg12)GQYCFADPYMC | 384 |
| BCY7241 | 54 | Ac-(74-01-04)Lys4(PEG12) | Ac-CIEEK(Peg12)QYCFADPYMC | >>3000 |
| BCY7242 | 55 | Ac-(74-01-04)Lys5(PEG12) | Ac-CIEEGK(Peg12)YCFADPYMC | 48.3 |
| BCY7243 | 56 | Ac-(74-01-04)Lys6(PEG12) | Ac-CIEEGQK(Peg12)CFADPYMC | >>3000 |
| BCY7244 | 57 | Ac-(74-01-04)Lys7(PEG12) | Ac-CIEEGQYCK(Peg12)ADPYMC | 296 |
| BCY7245 | 58 | Ac-(74-01-04)Lys8(PEG12) | Ac-CIEEGQYCFK(Peg12)DPYMC | 777 |
| BCY7246 | 59 | Ac-(74-01-04)Lys9(PEG12) | Ac-CIEEGQYCFAK(Peg12)PYMC | >>3000 |
| BCY7247 | 60 | Ac-(74-01-04)Lys10(PEG12) | Ac-CIEEGQYCFADK(Peg12)YMC | 239 |
| BCY7248 | 61 | Ac-(74-01-04)Lys11(PEG12) | Ac-CIEEGQYCFADPK(Peg12)MC | 744 |
| BCY7249 | 62 | Ac-(74-01-04)Lys12(PEG12) | Ac-CIEEGQYCFADPYK(Peg12)C | 288 |
| BCY7416 | 63 | Ac-(74-01-04)D-Lys4(PEG12)Nle12 | [Ac]CIEE[dK(PEG12FI)]QYCFADPY[Nle]C | 50.5 |
| BCY7519 | 64 | (74-01-04) Nle12 | ACIEEGQYCFADPY[Nle]CA | 61 |
| BCY7520 | 65 | (Peg12)-(74-01-04) Nle12 | [Peg12]-ACIEEGQYCFADPY[Nle]CA | 121 |
| BCY642 | 66 | (74-01-05)-Sar6-K | ACIEEGQYCYADHQLCA-Sar6-K | 245.5 |
| BCY643 | 67 | (74-01-06)-Sar6-K | ACIEEGQYCHADPYYCA-Sar6-K | 148 |
| BCY644 | 68 | (74-01-07)-Sar6-K | ACIEEGQYCHADPYFCA-Sar6-K | 145 |
| BCY645 | 69 | (74-01-08)-Sar6-K | ACIEEGQYCYADHYMCA-Sar6-K | 146.5 |
| BCY646 | 70 | (74-01-09)-Sar6-K | ACIEEGQYCYADPYLCA-Sar6-K | 105 |
| BCY647 | 71 | (74-01-09-T03)-Sar6-K(FI) | ACIEEGQYCYADPYLCSVG-Sar6-K | 391.5 |
| BCY648 | 72 | (B-Ala)-Sar5-(74-01-09-T03) | (FI)G-Sar5-ACIEEGQYCYADPYLCSVG | 228 |
| BCY649 | 73 | (74-01-10)-Sar6-K | ACIEEGQYCYSDPYLCA-Sar6-K | 207 |
| BCY650 | 74 | (74-01-11)-Sar6-K | ACIEEGQYCFADPYLCA-Sar6-K | 86.5 |
| BCY652 | 75 | (74-01-13)-Sar6-K | ACIEEGQYCHADPYMCA-Sar6-K | 142 |
| BCY653 | 76 | (74-01-14)-Sar6-K | ACIEEGQYCHADPQMCA-Sar6-K | 383 |
| BCY655 | 77 | (74-01-16)-Sar6-K | ACDIGPPYCYRDMYMCA-Sar6-K | 1337 |
| BCY656 | 78 | (74-01-17)-Sar6-K | ADIGPPYCYADPYMCA-Sar6-K | 240 |
| BCY7251 | 79 | (74-01-19-N002)-Sar6-K | ACLDPGPFCFADPYMCA-Sar6-K | 193 |
| BCY7253 | 80 | (74-01-20-N002)-Sar6-K | ACLDEGPYCFADPYFCA-Sar6-K | 183 |
| BCY7255 | 81 | (74-01-21-N002)-Sar6-K | ACINEGPYCFADPYMCA-Sar6-K | 136 |
| BCY7257 | 82 | (74-01-22-N002)-Sar6-K | ACIEQGPFCFADPYMCA-Sar6-K | 109 |
| BCY7259 | 83 | (74-01-23-N002)-Sar6-K | ACVEEGPFCFADPYYCA-Sar6-K | 105 |
| BCY7261 | 84 | (74-01-24-N002)-Sar6-K | ACLDEGPFCFSDPYMCA-Sar6-K | 453 |
| BCY657 | 85 | (B-Ala)-Sar5-(74-02-00) | [B-Ala]-Sar5-ACDEWGLFCIPHSDCA | 3621 |
| BCY659 | 86 | (74-02-01)-Sar6-K | ACDEWGLYCFAHPDCA-Sar6-K | 1041 |
| BCY7119 | 87 | (74-13-00-T02)-Sar6-K | ACLDPGPYCYADPYMCTFH-Sar6-K | 144 |
| BCY660 | 88 | (74-19-00-T01)-Sar6-K | ACIEPGPFCYADPYMCNRV-Sar6-K | 183.5 |
| BCY661 | 89 | (B-Ala)-Sar5-(74-19-00-T01) | G-Sar5-ACIEPGPFCYADPYMCNRV | 412 |
| BCY7120 | 90 | (74-20-00-T01)-Sar6-K | ACLEPGPYCYADPYMCTHL-Sar6-K | 160 |
| BCY7122 | 91 | (74-22-03-N004)-Sar6-K | ACLPPGPYCFPDPYFCA-Sar6-K | 147 |

TABLE 2
Alanine Scan Results

| Peptide | SEQ ID | | | |
|---|---|---|---|---|
| Number | NO: | Peptide Reference | Sequence | Kd nM |
| BCY7151 | 92 | [PEG3]-(74-01-04) Nle12 | [PEG3]-ACIEEGQYCFADPY[Nle]CA | 24.0 |
| BCY7283 | 93 | [PEG3]-(74-01-04) Ala1 Nle12 | [PEG3]-ACAEEGQYCFADPY(Nle)CA | 231 |
| BCY7284 | 94 | [PEG3]-(74-01-04) Ala2 Nle12 | [PEG3]-ACIAEGQYCFADPY(Nle)CA | 160 |
| BCY7285 | 95 | [PEG3]-(74-01-04) Ala3 Nle12 | [PEG3]-ACIEAGQYCFADPY(Nle)CA | 185 |
| BCY7286 | 96 | [PEG3]-(74-01-04) Ala4 Nle12 | [PEG3]-ACIEEAQYCFADPY(Nle)CA | 2568 |
| BCY7287 | 97 | [PEG3]-(74-01-04) Ala5 Nle12 | [PEG3]-ACIEEGAYCFADPY(Nle)CA | 25.5 |
| BCY7288 | 98 | [PEG3]-(74-01-04) Ala6 Nle12 | [PEG3]-ACIEEGQACFADPY(Nle)CA | 2322 |
| BCY7289 | 99 | [PEG3]-(74-01-04) Ala7 Nle12 | [PEG3]-ACIEEGQYCAADPY(Nle)CA | 904 |
| BCY7290 | 100 | [PEG3]-(74-01-04) Ala9 Nle12 | [PEG3]-ACIEEGQYCFAAPY(Nle)CA | >>3000 |
| BCY7292 | 101 | [PEG3]-(74-01-04) Ala11 Nle12 | [PEG3]-ACIEEGQYCFADPA(Nle)CA | 593 |
| BCY7293 | 102 | [PEG3]-(74-01-04) Ala12 | [PEG3]-ACIEEGQYCFADPYACA | 417 |
| BCY7294 | 103 | [PEG3]-(74-01-04) D-Ala1 Nle12 | [PEG3]-ACaEEGQYCFADPY(Nle)CA | >>3000 |
| BCY7295 | 104 | [PEG3]-(74-01-04) D-Ala2 Nle12 | [PEG3]-ACIaEGQYCFADPY(Nle)CA | >>3000 |
| BCY7296 | 105 | [PEG3]-(74-01-04) D-Ala3 Nle12 | [PEG3]-ACIEaGQYCFADPY(Nle)CA | >>3000 |
| BCY7297 | 106 | [PEG3]-(74-01-04) D-Ala4 Nle12 | [PEG3]-ACIEEaQYCFADPY(Nle)CA | 25.2 |
| BCY7298 | 107 | [PEG3]-(74-01-04) D-Ala5 Nle12 | [PEG3]-ACIEEGaYCFADPY(Nle)CA | 756 |
| BCY7299 | 108 | [PEG3]-(74-01-04) D-Ala6 Nle12 | [PEG3]-ACIEEGQaCFADPY(Nle)CA | >>3000 |
| BCY7300 | 109 | [PEG3]-(74-01-04) D-Ala7 Nle12 | [PEG3]-ACIEEGQYCaADPY(Nle)CA | >>3000 |
| BCY7301 | 110 | [PEG3]-(74-01-04) D-Ala8 Nle12 | [PEG3]-ACIEEGQYCFaDPY(Nle)CA | >>3000 |
| BCY7302 | 111 | [PEG3]-(74-01-04) D-Ala9 Nle12 | [PEG3]-ACIEEGQYCFAaPY(Nle)CA | >>3000 |
| BCY7303 | 112 | [PEG3]-(74-01-04) D-Ala10 Nle12 | [PEG3]-ACIEEGQYCFADaY(Nle)CA | 968 |
| BCY7304 | 113 | [PEG3]-(74-01-04) D-Ala11 Nle12 | [PEG3]-ACIEEGQYCFADPa(Nle)CA | >>3000 |
| BCY7305 | 114 | [PEG3]-(74-01-04) D-Ala12 | [PEG3]-ACIEEGQYCFADPYACA | >>3000 |

TABLE 3
Direct Binding Results with Selected Peptides

| Peptide | SEQ | | | |
|---|---|---|---|---|
| Number | ID NO: | Peptide Reference | Sequence | Kd nM |
| BCY7151 | 92 | [PEG3]-(74-01-04) Nle12 | [PEG3]-ACIEEGQYCFADPY[Nle]CA | 24.0 |
| BCY7152 | 115 | [PEG3]-(74-01-04) Leu1 Nle12 | [PEG3]-ACLEEGQYCFADPY[Nle]CA | 55.1 |
| BCY7153 | 116 | [PEG3]-(74-01-04) Niel Nle12 | [PEG3]-AC[Nle]EEGQYCFADPY[Nle]CA | 122.6 |
| BCY7154 | 117 | [PEG3]-(74-01-04) Chg1 Nle12 | [PEG3]-AC-Chg-EEGQYCFADPY[Nle]CA | 20.3 |
| BCY7155 | 118 | [PEG3]-(74-01-04) Cha1 Nle12 | [PEG3]-AC-Cha-EEGQYCFADPY[Nle]CA | 175.5 |
| BCY7156 | 119 | [PEG3]-(74-01-04) Pro2 Nle12 | [PEG3]-ACIPEGQYCFADPY[Nle]CA | 10.4 |
| BCY7157 | 120 | [PEG3]-(74-01-04) Asp2 Nle12 | [PEG3]-ACIDEGQYCFADPY[Nle]CA | 26.2 |
| BCY7158 | 121 | [PEG3]-(74-01-04) Aad2 Nle12 | [PEG3]-ACI-Aad-EGQYCFADPY[Nle]CA | 22.3 |
| BCY7159 | 122 | [PEG3]-(74-01-04) Api2 Nle12 | [PEG3]-ACI-Api-EGQYCFADPY[Nle]CA | 58.0 |
| BCY7160 | 123 | [PEG3]-(74-01-04) Pro3 Nle12 | [PEG3]-ACIEPGQYCFADPY[Nle]CA | 68.1 |
| BCY7161 | 124 | [PEG3]-(74-01-04) Asp3 Nle12 | [PEG3]-ACIEDGQYCFADPY[Nle]CA | 282.1 |
| BCY7162 | 125 | [PEG3]-(74-01-04) Aad3 Nle12 | [PEG3]-ACIE-Aad-GQYCFADPY[Nle]CA | 39.8 |
| BCY7163 | 126 | [PEG3]-(74-01-04) Api3 Nle12 | [PEG3]-ACIE-Api-GQYCFADPY[Nle]CA | 126.3 |
| BCY7164 | 127 | [PEG3]-(74-01-04) Sar4 Nle12 | [PEG3]-ACIEE-Sar-QYCFADPY[Nle]CA | 326.0 |
| BCY7165 | 128 | [PEG3]-(74-01-04) D-Lys4 Nle12 | [PEG3]-ACIEE-DLys-QYCFADPY[Nle]CA | 24.0 |
| BCY7166 | 129 | [PEG3]-(74-01-04) D-Phe4 Nle12 | [PEG3]-ACIEE-DPhe-QYCFADPY[Nle]CA | 10.0 |
| BCY7167 | 130 | [PEG3]-(74-01-04) D-G1u4 Nle12 | [PEG3]-ACIEE-DGIu-QYCFADPY[Nle]CA | 31.9 |
| BCY7168 | 131 | [PEG3]-(74-01-04) D-G1n4 Nle12 | [PEG3]-ACIEE-DGIn-QYCFADPY[Nle]CA | 15.7 |
| BCY7169 | 132 | [PEG3]-(74-01-04) D-Leu4 Nle12 | [PEG3]-ACIEE-DLeu-QYCFADPY[Nle]CA | 46.1 |
| BCY7170 | 133 | [PEG3]-(74-01-04) D-Ser4 Nle12 | [PEG3]-ACIEE-DSer-QYCFADPY[Nle]CA | 13.9 |
| BCY7172 | 134 | [PEG3]-(74-01-04) N-Me-D-Ala4 Nle12 | [PEG3]-ACIEE-MeDala-QYCFADPY[Nle]CA | 546.4 |
| BCY7173 | 135 | [PEG3]-(74-01-04) Aib4 Nle12 | [PEG3]-ACIEE-Aib-QYCFADPY[Nle]CA | 414.0 |
| BCY7174 | 136 | [PEG3]-(74-01-04) Pro5 Nle12 | [PEG3]-ACIEEGPYCFADPY[Nle]CA | 6.13 |
| BCY7175 | 137 | [PEG3]-(74-01-04) Phe6 Nle12 | [PEG3]-ACIEEGQFCFADPY[Nle]CA | 25.3 |
| BCY7176 | 138 | [PEG3]-(74-01-04) 2MePhe6 Nle12 | [PEG3]-ACIEEGQ-2MeF-CFADPY[Nle]CA | 88.4 |
| BCY7177 | 139 | [PEG3]-(74-01-04) 3MePhe6 Nle12 | [PEG3]-ACIEEGQ-3MeF-CFADPY[Nle]CA | 43.7 |
| BCY7178 | 140 | [PEG3]-(74-01-04) 4MePhe6 Nle12 | [PEG3]-ACIEEGQ-4MeF-CFADPY[Nle]CA | 21.8 |
| BCY7179 | 141 | [PEG3]-(74-01-04) 4FPhe6 Nle12 | [PEG3]-ACIEEGQ-4FF-CFADPY[Nle]CA | 30.5 |
| BCY7180 | 142 | [PEG3]-(74-01-04) 3FPhe6 Nle12 | [PEG3]-ACIEEGQ-3FF-CFADPY[Nle]CA | 54.4 |
| BCY7181 | 143 | [PEG3]-(74-01-04) 2MePhe7 Nle12 | [PEG3]-ACIEEGQYC-2MeF-ADPY[Nle]CA | 86.4 |
| BCY7182 | 144 | [PEG3]-(74-01-04) 3MePhe7 Nle12 | [PEG3]-ACIEEGQYC-3MeF-ADPY[Nle]CA | 63.3 |
| BCY7183 | 145 | [PEG3]-(74-01-04) 4MePhe7 Nle12 | [PEG3]-ACIEEGQYC-4MeF-ADPY[Nle]CA | 34.2 |
| BCY7184 | 146 | [PEG3]-(74-01-04) Phg7 Nle12 | [PEG3]-ACIEEGQYC-PheG-ADPY[Nle]CA | 2813.3 |
| BCY7185 | 147 | [PEG3]-(74-01-04) 4FPhe7 Nle12 | [PEG3]-ACIEEGQYC-4FF-ADPY[Nle]CA | 19.6 |
| BCY7186 | 148 | [PEG3]-(74-01-04) Gly8 Nle12 | [PEG3]-ACIEEGQYCFGDPY[Nle]CA | 244.2 |
| BCY7187 | 149 | [PEG3]-(74-01-04) Ser8 Nle12 | [PEG3]-ACIEEGQYCFSDPY[Nle]CA | 83.9 |
| BCY7188 | 150 | [PEG3]-(74-01-04) Pro8 Nle12 | [PEG3]-ACIEEGQYCFPDPY[Nle]CA | 363.1 |
| BCY7189 | 151 | [PEG3]-(74-01-04) Asn8 Nle12 | [PEG3]-ACIEEGQYCFANPY[Nle]CA | 655.8 |
| BCY7190 | 152 | [PEG3]-(74-01-04) Pip10 Nle12 | [PEG3]-ACIEEGQYCFAD-Pip-Y[Nle]CA | 326.8 |
| BCY7191 | 153 | [PEG3]-(74-01-04) N-Me-Ala10 Nle12 | [PEG3]-ACIEEGQYCFAD-MeAla-Y[Nle]CA | 460.2 |
| BCY7192 | 154 | [PEG3]-(74-01-04) Sar10 Nle12 | [PEG3]-ACIEEGQYCFAD-Sar-Y[Nle]CA | 220.6 |
| BCY7193 | 155 | [PEG3]-(74-01-04) Aib10 Nle12 | [PEG3]-ACIEEGQYCFAD-Aib-Y[Nle]CA | 146.9 |
| BCY7195 | 156 | [PEG3]-(74-01-04) tBuAla1 Nle12 | [PEG3]AC[tBuAla]EEGQYCFADPY[Nle]CA | 15.7 |
| BCY7196 | 157 | [PEG3]-(74-01-04) HLeu1 Nle12 | [PEG3]AC[HLeu]EEGQYCFADPY[Nle]CA | 138.5 |
| BCY7197 | 158 | [PEG3]-(74-01-04) 2FPhe6 Nle12 | [PEG3]ACIEEGQ[2FPhe]CFADPY[Nle]CA | 95.1 |
| BCY7198 | 159 | [PEG3]-(74-01-04) 2FPhe7 Nle12 | [PEG3]ACIEEGQYC[2FPhe]ADPY[Nle]CA | 28.1 |
| BCY7199 | 160 | [PEG3]-(74-01-04) CF3G8 Nle12 | [PEG3]ACIEEGQYCF[CF3G]DPY[Nle]CA | 172.0 |
| BCY7200 | 161 | [PEG3]-(74-01-04) pCoPhe11 Nle12 | [PEG3]ACIEEGQYCFADP[pCoPhe][Nle]CA | 364.6 |
| BCY7201 | 162 | [PEG3]-(74-01-04) pCaPhe11 Nle12 | [PEG3]ACIEEGQYCFADP[pCaPhe][Nle]CA | 533.4 |
| BCY7202 | 163 | [PEG3]-(74-01-04) Gln11 Nle12 | [PEG3]ACIEEGQYCFADPQ[Nle]CA | 216.0 |
| BCY7205 | 164 | [PEG3]-(74-01-04) 2MePhe11 Nle12 | [PEG3]ACIEEGQYCFADP[2MePhe][Nle]CA | 147.3 |
| BCY7206 | 165 | [PEG3]-(74-01-04) 3MePhe11 Nle12 | [PEG3]ACIEEGQYCFADP[3MePhe][Nle]CA | 157.8 |
| BCY7207 | 166 | [PEG3]-(74-01-04) 4MePhe11 Nle12 | [PEG3]ACIEEGQYCFADP[4MePhe][Nle]CA | 185.8 |
| BCY7208 | 167 | [PEG3]-(74-01-04) Cit11 Nle12 | [PEG3]ACIEEGQYCFADP[Cit][Nle]CA | 657.4 |
| BCY7209 | 168 | [PEG3]-(74-01-04) 4FPhe11 Nle12 | [PEG3]ACIEEGQYCFADP[4FPhe][Nle]CA | 154.1 |
| BCY7210 | 169 | [PEG3]-(74-01-04) tBuAla12 | [PEG3]ACIEEGQYCFADPY[tBuAla]CA | 70.0 |
| BCY7211 | 170 | [PEG3]-(74-01-04) HLeu12 | [PEG3]ACIEEGQYCFADPY[HLeu]CA | 39.1 |
| BCY7212 | 171 | [PEG3]-(74-01-04)Ile12 | [PEG3]ACIEEGQYCFADPYICA | 140.3 |
| BCY7213 | 172 | [PEG3]-(74-01-04) Cha12 | [PEG3]ACIEEGQYCFADPY[Cha]CA | 62.1 |
| BCY7214 | 173 | [PEG3]-(74-01-04) Phe12 | [PEG3]ACIEEGQYCFADPYFCA | 50.1 |
| BCY7215 | 174 | [PEG3]-(74-01-04) 2MePhe12 | [PEG3]ACIEEGQYCFADPY[2MePhe]CA | 58.2 |
| BCY7216 | 175 | [PEG3]-(74-01-04) 3MePhe12 | [PEG3]ACIEEGQYCFADPY[3MePhe]CA | 88.0 |
| BCY7217 | 176 | [PEG3]-(74-01-04) 4MePhe12 | [PEG3]ACIEEGQYCFADPY[4MePhe]CA | 134.2 |
| BCY7218 | 177 | [PEG3]-(74-01-04) Cys1Pen Nle12 | [PEG3]A[Pen]IEEGQYCFADPY[Nle]CA | 40.7 |
| BCY7219 | 178 | [PEG3]-(74-01-04) Cys2Pen Nle12 | [PEG3]ACIEEGQY[Pen]FADPY[Nle]CA | 482.1 |
| BCY7220 | 179 | [PEG3]-(74-01-04) Cys3Pen Nle12 | [PEG3]ACIEEGQYCFADPY[Nle][Pen]A | 2465.1 |
| BCY7221 | 180 | [PEG3]-(74-01-04) Cys1HCys Nle12 | [PEG3]A[HCys]IEEGQYCFADPY[Nle]CA | 50.8 |
| BCY7222 | 181 | [PEG3]-(74-01-04) Cys2HCys Nle12 | [PEG3]ACIEEGQY[HCys]FADPY[Nle]CA | 1493.1 |
| BCY7223 | 182 | [PEG3]-(74-01-04) Cys3HCys Nle12 | [PEG3]ACIEEGQYCFADPY[Nle][HCys]A | 279.6 |
| BCY7224 | 183 | [PEG3]-(74-01-04) 3FPhe7 Nle12 | [PEG3]ACIEEGQYC[3FPhe]ADPY[Nle]CA | 39.8 |
| BCY7306 | 184 | [PEG3]-(74-01-04) TetraZ2 Nle12 | [PEG3]-ACI[TetraZ]EGQYCFADPY[Nle]CA | 289.3 |
| BCY7308 | 185 | [PEG3]-(74-01-04) TetraZ9 Nle12 | [PEG3]-ACIEEGQYCFA[TetraZ]PY[Nle]CA | 842.1 |
| BCY7309 | 186 | [PEG3]-(74-01-04) HGIn11 Nle12 | [PEG3]-ACIEEGQYCFADP[HGIn][Nle]CA | 536.9 |
| BCY7310 | 187 | [PEG3]-(74-01-04) Ahp1 Nle12 | [PEG3]-AC+AhNEEGQYCFADPY[Nle]CA | 278.5 |
| BCY7311 | 188 | [PEG3]-(74-01-04) Ahp12 | [PEG3]-ACIEEGQYCFADPY[Ahp]CA | 19.2 |

### 2. CD137 Biacore Experiments

### (a) Amine Coupled CD137 Target Assay Description

Biacore experiments were performed to determine kₐ (M⁻¹s⁻¹), k_{d} (s⁻¹), K_{D} (nM) values of peptides binding to human CD137 (AcroBiosystems) protein. CD137 protein was diluted and immobilised using the standard amine coupling procedure to chip CM5 (#BR-1005-30). CD137 protein was diluted to 10 μg/ml in NaAc pH 5.5 and used for coupling. Ethanolamine is then injected to deactivate remaining active esters.

The CD137 protein was immobilise at 180 RUs of CD137 protein to generate the maximum theoretical binding response with a peptide of 2500 MW will be ˜25 RUs. A blank immobilisation of the reference flow cell (Fc1 or Fc3) is performed when amine coupling, following exactly the same procedure but with no injection of protein target. The peptides were tested at starting concentrations of 300-450 nM and diluted in ½ dilutions series. The DMSO concentration was adjusted to remain constant.

The peptide binding kinetic analysis was performed as follows at flow rate 50 μl/min, 200 sec association, 600 sec dissociation and 60 sec stabilization. The Bicyclic peptides were fitted using the 1:1 model Biacore T200 Evaluation software.

Selected peptides of the invention were tested in the above mentioned assay and the results are shown in Table 4:

TABLE 4
SPR Data for Selected Peptides
of the Invention

| | SEQ | | | |
|---|---|---|---|---|
| Peptide | ID | Peptide | | |
| Number | NO: | Reference | Sequence | K_{D} nM |
| BCY592 | 189 | 74-01-04 | ACIEEGQYCFADPYMCA | 70.9 |
| BCY589 | 190 | 74-01-01- | HEHCIEEGQYCYADPYM | 124.1 |
| | | T01 | CA | |
| BCY599 | 191 | 74-01-11 | ACIEEGQYCFADPYLCA | 191.4 |
| BCY631 | 192 | 74-22-03 | ACLPPGPYCFPDPYFCA | 92.3 |

### (b) Biotinylated CD137 Target Assay Description

Biacore experiments were performed to determine kₐ (M⁻¹s⁻¹), k_{d} (s⁻¹), K_{D} (nM) values of peptides binding to human CD137 protein. Recombinant human CD137 homotrimer (R&D systems) was resuspended in PBS and biotinylated using EZ-Link™ Sulfo-NHS-LC-LC-Biotin reagent (Thermo Fisher) as per the manufacturer's suggested protocol. The protein was desalted to remove uncoupled biotin using spin columns into PBS.

For analysis of binding, a Biacore T200 instrument was used utilising a XanTec CMD500D chip. Streptavidin was immobilized on the chip using standard amine-coupling chemistry at 25° C. with HBS-N (10 mM HEPES, 0.15 M NaCl, pH 7.4) as the running buffer. Briefly, the carboxymethyl dextran surface was activated with a 7 min injection of a 1:1 ratio of 0.4 M 1-ethyl-3-(3-dimethylaminopropyl) carbodiimide hydrochloride (EDC)/0.1 M N-hydroxy succinimide (NHS) at a flow rate of 10 μl/min. For capture of streptavidin, the protein was diluted to 0.2 mg/ml in 10 mM sodium acetate (pH 4.5) and captured by injecting 120 μl of onto the activated chip surface. Residual activated groups were blocked with a 7 min injection of 1 M ethanolamine (pH 8.5) and biotinylated CD137 captured to a level of 800-1800 RU. Buffer was changed to PBS/0.05% Tween 20 and a dilution series of the peptides was prepared in this buffer with a final DMSO concentration of 0.5%. The top peptide concentration was 500 nM or 10 μM with 6 further 3-fold (500 nm), or 2-fold (10 μM) dilutions in PBS/0.05% Tween 20. The SPR analysis was run at 25° C. at a flow rate of 90 μl/min with 60 seconds association and 100-600 seconds dissociation. After each cycle, a regeneration step (10 μl of 10 mM glycine pH 2) was employed. Data were corrected for DMSO excluded volume effects. All data were double-referenced for blank injections and reference surface using standard processing procedures and data processing and kinetic fitting were performed using Scrubber software, version 2.0c (BioLogic Software). Data were fitted using mass transport model allowing for mass transport effects where appropriate.

Selected peptides of the invention were tested in the above mentioned assay and the results are shown in Table 5:

TABLE 5
SPR Data for Selected Peptides of the Invention

| Peptide | SEQ ID | | | |
|---|---|---|---|---|
| Number | NO: | Peptide Reference | Sequence | KD nM |
| BCY592 | 189 | 74-01-04 | ACIEEGQYCFADPYMCA | 41.8 |
| BCY593 | 193 | Ac-(74-01-04) | [Ac]CIEEGQYCFADPYMC | 37 |
| BCY3814 | 31 | 74-01-04 Nle12 | ACIEEGQYCFADPY(Nle)CA | 33.3 |
| BCY7527 | 194 | Ac-(74-01-04)-Dap Nle12 | [Ac]CIEEGQYCFADPY[Nle]C[Dap] | 16.4 |
| BCY7768 | 195 | PEG3-(74-01-04) Pro2 D-Phe4 Nle12 | [PEG3]ACIPE[dF]QYCFADPY[Nle]CA | 33.9 |
| BCY7770 | 196 | PEG3-(74-01-04) Pro2 D-Phe4 Pro5 | [PEG3]ACIPE[dF]PYCFADPY[Nle]CA | 18.6 |
| | | Nle12 | | |
| BCY7772 | 197 | PEG3-(74-01-04) D-Phe4 Pro5 Nle12 | [PEG3]ACIEE[dF]PYCFADPY[Nle]CA | 39.7 |
| BCY7773 | 198 | PEG3-(74-01-04) Pro2 Pro5 Nle12 | PEG3]ACIPEGPYCFADPY[Nle]CA | 31.5 |
| BCY7774 | 199 | PEG3-(74-01-04) tBuAla1 Pro2 D- | PEG3]AC[tBuAla]PE[dF]P[4MePhe]C | 8.69 |
| | | Phe4 Pro5 4MePhe6 4FPhe7 Nle12 | [4FPhe]ADPY[Nle]CA | |
| BCY7775 | 200 | PEG3-(74-01-04) tBuAla1 Pro2 D- | [PEG3]AC[tBuAla]PE[dF]Q[4MePhe] | 18.2 |
| | | Phe4 4MePhe6 4FPhe7 Nle12 | C[4FPhe]ADPY[Nle]CA | |
| BCY7776 | 201 | PEG3-(74-01-04) tBuAla1 D-Phe4 | [PEG3]AC[tBuAla]EE[dF]P[4MePhe] | 17.3 |
| | | Pro5 4MePhe6 4FPhe7 Nle12 | C[4Fphe]ADPY[Nle]CA | |
| BCY7777 | 202 | PEG3-(74-01-04) tBuAla1 D-Phe4 | [PEG3]AC[tBuAla]EE[dF]Q[4MePhe]C | 66 |
| | | 4MePhe6 4FPhe7 Nle12 | [4FPhe]ADPY(Nle]CA | |
| BCY7796 | 203 | PEG3-(74-01-04) HyP2 Nle12 | [PEG3]ACI[HyP]EGQYCFADPY[Nle]CA | 24.7 |
| BCY7798 | 204 | PEG3-(74-01-04) D-Trp4 Nle12 | [PEG3]ACIEE[dW]QYCFADPY[Nle]CA | 12.1 |
| BCY7799 | 205 | PEG3-(74-01-04) Aze5 Nle12 | [PEG3]ACIEEG[Aze]YCFADPY[Nle]CA | 69.9 |
| BCY7800 | 206 | PEG3-(74-01-04) Pip5 Nle12 | [PEG3]ACIEEG[Pip]YCFADPY[Nle]CA | 1490 |
| BCY7801 | 207 | PEG3-(74-01-04) 2Nal6 Nle12 | PEG3]ACIEEGQ[2Nal]CFADPY[Nle]CA | 18.7 |
| BCY7802 | 208 | PEG3-(74-01-04) 4MeOPhe6 Nle12 | PEG3]ACIEEGQ[4MeoPhe]CFADPY | 17.8 |
| | | | [Nle]CA | |
| BCY7803 | 209 | PEG3-(74-01-04) Tyr6 Nle12 | [PEG3]ACIEEGQYCYADPY[Nle]CA | 54.9 |
| BCY7804 | 210 | PEG3-(74-01-04) Aze10 Nle12 | [PEG3]ACIEEGQYCFAD[Aze]Y[Nle]CA | 85.7 |
| BCY7806 | 211 | PEG3-(74-01-04) Hse(Me)12 | [PEG3]ACIEEGQYCFADPY[Hse(Me)]CA | 204 |
| BCY7923 | 212 | Ac-(74-01-04) NMelle1 Nle12 | [Ac]AC[NMelle]EEGQYCFADPY[Nle]CA | 1149 |
| BCY7924 | 213 | Ac-(74-01-04) Aze2 Nle12 | [Ac]ACI[Aze]EGQYCFADPY[Nle]CA | 59 |
| BCY7925 | 214 | Ac-(74-01-04) Pip2 Nle12 | [Ac]ACI[Pip]EGQYCFADPY[Nle]CA | 105 |
| BCY7926 | 215 | Ac-(74-01-04) NMeGlu2 Nle12 | [Ac]ACI[NMeGlu]EGQYCFADPY(Nle]CA | 220 |
| BCY7927 | 216 | Ac-(74-01-04) NMeGlu3 Nle12 | [Ac]ACIE[NMeGlu]GQYCFADPY[Nle]CA | 1650 |
| BCY7928 | 217 | Ac-(74-01-04) D-Asp4 Nle12 | [Ac]ACIEE[dD]QYCFADPY[Nle]CA | 97 |
| BCY7929 | 218 | Ac-(74-01-04) NMeAla5 Nle12 | [Ac]ACIEEG[NMeAla]YCFADPY[Nle]CA | 269 |
| BCY7930 | 219 | Ac-(74-01-04) NMeTyr6 Nle12 | Ac]ACIEEGQ[NMeTyr]CFADPY[Nle]CA | 993 |
| BCY7931 | 220 | Ac-(74-01-04) HPhe6 Nle12 | [Ac]ACIEEGQ[HPhe]CFADPY[Nle]CA | 1746 |
| BCY7933 | 221 | Ac-(74-01-04) 2Pal6 Nle12 | [Ac]ACIEEGQ[2Pal]CFADPY[Nle]CA | 790 |
| BCY7934 | 222 | Ac-(74-01-04) 3Pal6 Nle12 | [Ac]ACIEEGQ[3Pal]CFADPY[Nle]CA | 196 |
| BCY7936 | 223 | Ac-(74-01-04) 4,4-BPA6 Nle12 | [Ac]ACIEEGQ[44BPA]CFADPY[Nle]CA | 43 |
| BCY7937 | 224 | Ac-(74-01-04) HPhe7 Nle12 | [Ac]ACIEEGQYC[HPhe]ADPY[Nle]CA | 556 |
| BCY7939 | 225 | Ac-(74-01-04) 2Pal7 Nle12 | [Ac]ACIEEGQYC[2Pal]ADPY[Nle]CA | 98.6 |
| BCY7940 | 226 | Ac-(74-01-04) 3Pal7 Nle12 | [Ac]ACIEEGQYC[3Pal]ADPY[Nle]CA | 58.6 |
| BCY7941 | 227 | Ac-(74-01-04) 4Pal7 Nle12 | [Ac]ACIEEGQYC[4Pal]ADPY(Nle]CA | 44.4 |
| BCY7942 | 228 | Ac-(74-01-04) 4,4-BPA7 Nle12 | [Ac]ACIEEGQYC[44BPA]ADPY[Nle]CA | 35.9 |
| BCY7943 | 229 | Ac-(74-01-04) 1Nal7 Nle12 | [Ac]ACIEEGQYC[1Nal]ADPY[Nle]CA | 151 |
| BCY7944 | 230 | Ac-(74-01-04) 4tBuPhe7 Nle12 | Ac]ACIEEGQYC[4tBuPhe]ADPY[Nle]CA | 42.2 |
| BCY7945 | 231 | Ac-(74-01-04) NMeAla8 Nle12 | [Ac]ACIEEGQYCF[NMeAla]DPY[Nle]CA | 665 |
| BCY7950 | 232 | Ac-(74-01-04) 5,5-dmP5 Nle12 | [Ac]ACIEEG(55DMP]YCFADPY[Nle]CA | 31.1 |
| BCY7953 | 233 | Ac-(74-01-04) HyP10 Nle12 | [Ac]ACIEEGQYCFAD[HyP]Y[Nle]CA | 86.6 |
| BCY7954 | 234 | Ac-(74-01-04) Oic5 Nle12 | [Ac]ACIEEG[Oic]YCFADPY[Nle]CA | 11.1 |
| BCY7955 | 235 | Ac-(74-01-04) Oic10 Nle12 | [Ac]ACIEEGQYCFAD[Oic]Y[Nle]CA | 169 |
| BCY7956 | 236 | Ac-(74-01-04) Oic2 Nle12 | [Ac]ACI[Oic]EGQYCFADPY[Nle]CA | 228 |
| BCY7957 | 237 | Ac-(74-01-04) Oxa10 Nle12 | [Ac]ACIEEGQYCFAD[Oxa]Y[Nle]CA | 118 |
| BCY7958 | 238 | Ac-(74-01-04) Oxa2 Nle12 | [Ac]ACI[Oxa]EGQYCFADPY[Nle]CA | 20 |
| BCY7959 | 239 | Ac-(74-01-04) Oxa5 Nle12 | [Ac]ACIEEG[Oxa]YCFADPY[Nle]CA | 37.7 |
| BCY7960 | 240 | Ac-(74-01-04) Pro2 Pro5 Nle12 | [Ac]ACIPEGPYCFADPY[Nle]CA | 10.7 |
| BCY7952 | 241 | Ac-(74-01-04) HyP5 Nle12 | [Ac]ACIEEG[HyP]YCFADPY[Nle]CA | 11.8 |
| BCY7961 | 242 | Ac-(74-01-04) Pro2 DAla4 | [Ac]ACIPE[dA]PYCFADPY[Nle]CA | 10 |
| | | Pro5 Nle12 | | |
| BCY7965 | 243 | Ac-(74-01-04) tBuAla1 Pro2 | [Ac]AC[tBuAla]PE[dA]PYCFADPY | 4.75 |
| | | DAla4 Pro5 Nle12 | [Nle]CA | |
| BCY8217 | 244 | A-(74-01-04)-A D-Ala8 Nle12 | ACIEEGQYCF[dA]DPY[Nle]CA | 500 |
| BCY8656 | 245 | Ac-(74-01-04) tBuAla1 Nle12 | [Ac]AC[tBuAla]EEGQYCFADPY[Nle] | 31 |
| | | | CA | (n = 2) |
| BCY8657 | 246 | Ac-(74-01-04) Chg1 Nle12 | [Ac]AC[Chg]EEGQYCFADPY[Nle]CA | 62.4 |
| BCY8658 | 247 | Ac-(74-01-04) Ac5c1 Nle12 | [Ac]AC[AC5C]EEGQYCFADPY[Nle]CA | 200 |
| BCY8659 | 248 | Ac-(74-01-04) Pro2 Nle12 | [Ac]ACIPEGQYCFADPY[Nle]CA | 33.4 |
| BCY8660 | 249 | Ac-(74-01-04) Gln2 Nle12 | [Ac]ACIQEGQYCFADPY[Nle]CA | 380 |
| BCY8661 | 250 | Ac-(74-01-04) Pro3 Nle12 | (Ac]ACIEPGQYCFADPY[Nle]CA | 154.5 |
| | | | | (n = 2) |
| BCY8662 | 251 | Ac-(74-01-04) Gln3 Nle12 | [Ac]ACIEQGQYCFADPY[Nle]CA | 179 |
| BCY8663 | 252 | Ac-(74-01-04) D-Phe4 Nle12 | [Ac]ACIEE[dF]QYCFADPY[Nle]CA | 25.1 |
| | | | | (n = 2) |
| BCY8664 | 253 | Ac-(74-01-04) D-Ala4 Nle12 | [Ac]ACIEE[dA]QYCFADPY[Nle]CA | 59.5 |
| | | | | (n = 2) |
| BCY8665 | 254 | Ac-(74-01-04) Ac5c4 Nle12 | [Ac]ACIEE[AC5C]QYCFADPY[Nle]CA | 200 |
| | | | | (n = 2) |
| BCY8667 | 255 | Ac-(74-01-04) Ala5 Nle12 | [Ac]ACIEEGAYCFADPY[Nle]CA | 68.5 |
| | | | | (n = 3) |
| BCY8668 | 256 | Ac-(74-01-04) Aib5 Nle12 | [Ac]ACIEEG[Aib]YCFADPY[Nle]CA | 28.7 |
| BCY8669 | 257 | Ac-(74-01-04) Ac5c5 Nle12 | [Ac]ACIEEG[AC5C]YCFADPY[NlelCA | 33.2 |
| | | | | (n = 2) |
| BCY8670 | 258 | Ac-(74-01-04) 4MePhe6 Nle12 | [Ac]ACIEEGQ[4MePhe]CFADPY[Nle]CA | 1000 |
| BCY8671 | 259 | Ac-(74-01-04) 1Nal6 Nle12 | [Ac]ACIEEGQ[1Nal]CFADPY[Nle]CA | 297 |
| BCY8673 | 260 | Ac-(74-01-04) 2Nal7 Nle12 | [Ac]ACIEEGQYC[2Nal]ADPY[Nle]CA | 117 |
| BCY8674 | 261 | Ac-(74-01-04) 4NO2Phe7 Nle12 | [Ac]ACIEEGQYC[NO2Phe]ADPY[Nle]CA | 44.5 |
| | | | | (n = 2) |
| BCY8675 | 262 | Ac-(74-01-04) 4BrPhe7 Nle12 | [Ac]ACIEEGQYC[4BrPhe]ADPY[Nle]CA | 57.5 |
| | | | | (n = 2) |
| BCY8676 | 263 | Ac-(74-01-04) Abu8 Nle12 | [Ac]ACIEEGQYCF[Abu]DPY[Nle]CA | 1000 |
| BCY8677 | 264 | Ac-(74-01-04) Ahp12 | [Ac]ACIEEGQYCFADPY[Ahp]CA | 64.6 |
| | | | | (n = 2) |
| BCY9273 | 265 | Ac-A-(74-01-04)-A | [Ac]ACIEEGQYCFADPYMCA | 108 |

### 3. CD137 Cell Activity

The biological activity of the CD137-specific peptides was tested using the cellular CD137 luciferase reporter assay kit (Promega). The cells in this commercially available kit express luciferase that is activated down-stream of CD137. This assay can be used to assess agonism (exemplified by CD137 ligand, CD137L) and antagonism (exemplified by bicyclic peptide 74-01-04-N002).

The Promega CD137 cell-activity assay uses NF-KB luciferase luminescence as a read-out of CD137 activation in Jurkat cells. Briefly, the experiments were performed by preparing medium by thawing FBS and adding 1% FBS to RPMI-1640 (Promega kit CS196005). Dilute agonists at concentration giving agonism CD137L (R&D systems 2295-4L/CF) diluted to 100 nM in the RPMI-1640 medium as final concentration in the assay. Dilute and then titrate down the bicyclic peptide in a sterile 96 well-plate. Suggested starting concentration for the bicyclic peptide is 10 μM, 100-fold excess over the agonist CD137L. Prepare enough reagent for duplicate samples and then perform ⅓ dilution series dilution series. Include positive control CD137L and bicyclic peptide alone. Thaw CD137 Jurkat cells in the water-bath and then add 500 μl cells to 9.5 ml pre-warmed 1% FBS RPMI-1640 medium. Add 50 μl cells/well to white cell culture plate. Add 12.5 μl bicyclic peptide (at 6× final concentration) to the cells. Then add 12.5 ul of agonist (at 6× final concentration) as duplicate samples or 1% FBS RPMI-1640 alone as background control.

Co-incubate cells together with CD137L agonist and bicyclic peptide for 6 h at 37° C., 5% CO₂. After 6 h thaw Bio-Glo™ and develop the assay at room-temperature. Add 75 μl Bio-Glo™ per well and incubate 5-10 min. Read luminescence signal on Pherastar plate-reader LUM plus models, gain 3600 using MARS software. Analyse data by calculating the percentage inhibition compared to CD137L alone. Transform the data to x=log (X), then plot log (inhibitor) vs. response variable slope (4 parameters) to calculate the IC₅₀ value.

The Promega CD137 cell-reporter assay (product number CS196008) was used to determine the antagonistic effect of the peptide BCY592 (74-01-04-N002; SEQ ID NO: 189) in inhibiting the natural ligand CD137L induction. The CD137 assay cells were co-incubated with trimeric CD137L (R&D systems)+BCY592 peptide. The CD137 reporter activity was determined as NF-KB promotor driven luciferase activity. The effect of the peptide BCY592 (SEQ ID NO: 189) was plotted as % inhibition relative to baseline CD137L activity in the assay and used to determine the IC50-value.

The results are shown in FIG. 1 where it can be seen that the bicyclic peptide BCY592(SEQ ID NO: 189) specific for CD137 can act as an antagonist that inhibits CD137L activity. This result indicates that this peptide can be used in settings where it is desirable to block CD137 biological activity. It is known that CD137 activity can cause liver injury due to inflammation driven by the local immune cells. It is therefore believed that the bicyclic peptide BCY592 (SEQ ID NO: 189) (and by inference other bicyclic CD137 peptides of the invention) may reduce CD137—CD137L driven inflammation which would reduce hepatotoxicity of CD137 agonists.

### 4. Fluorescence Polarization Competition Binding Assay

The binding site of the hCD137-specific Bicycle peptide was determined by competition experiment between a fluorescent labelled CD137 binding peptide and natural ligand CD137L, agonistic antibodies Urelumab and Utomilumab. Urelumab antibody binds to a distinct binding site while CD137L and Utomilumab both bind to the site termed the ligand-binding site.

The competitor agonists CD137L (R&D systems), Urelumab and Utomilumab were diluted in assay buffer 20 mM HEPES, 150 mM NaCl, 0.05% P20, pH7.5 to a top concentration of 500-1000 nM. The human CD137 protein (AcroBiosystems) was diluted to 500 nM final concentration in the assay. Finally, the fluorescent tracer peptide BCY640 (74-01-04-N001; SEQ ID NO: 49) was added at 1 nM. The assay was typically set up by adding 5 μL agonist competitor, 10 μL CD137 protein then 10 μL fluorescent peptide. The total volume of 25 μL was prepared in black walled and bottomed low binding low volume 384 well plates. Measurements were conducted on a BMG PHERAstar FS equipped with an FP 485 520 520 optic module at 25° C. with 200 flashes per well and a positioning delay of 0.1 second. Each well was measured every 5 minutes for 60 minutes. The gain was set in a well containing tracer without target protein. The mP-values at the end of the 60 minutes read were plotted against concentration of the agonists. Reduction in the mP-values indicates competition between the known agonist and the tracer peptide.

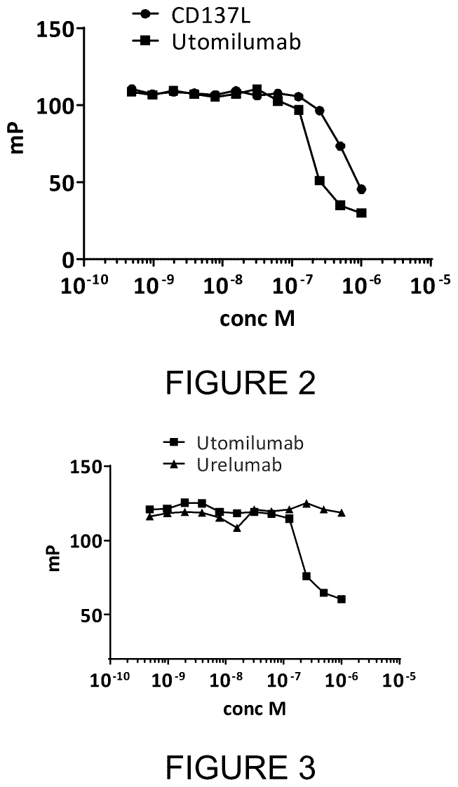

The results are shown in FIGS. 2 and 3 where it can be seen that the CD137 binding Bicycle (BCY640; SEQ ID NO: 49) binds to the physiologically relevant epitope that is shared with both the natural CD137 ligand (CD137L) and CD137 antibody (Utomilumab).

1. A peptide ligand specific for CD137, or a pharmaceutically acceptable salt thereof. comprising a polypeptide, or a pharmaceutically acceptable salt thereof, wherein said polypeptide comprises at least three reactive groups, separated by at least two loop sequences, and a molecular scaffold which forms covalent bonds with the reactive groups of the polypeptide such that at least two polypeptide loops are formed on the molecular scaffold;
wherein the peptide ligand comprises an amino acid sequence comprising SEQ ID NO: 266, or a modified derivative thereof:
(SEQ ID NO: 266)
Cᵢ-X₅-X₆-X₇-X₈-X₉-X₁₀-Cᵢᵢ-X₁₁-
X₁₂-D-X₁₃-X₁₄-X₁₅-Cᵢᵢᵢ;
wherein Cᵢ, Cᵢᵢ and Cᵢᵢᵢ represent first, second and third cysteine residues, respectively;
X₅ represents Ile, tBuAla or Chg;
X₆ represents Glu, Pro, Asp, Lys, Aad, HyP or Oxa;
X₇ represents Glu, Lys or Aad;
X₈ represents Gly, D-Lys, D-Ala, L-Ala, D-Phe, D-Glu, D-Gln, D-Leu, D-Ser or D-Trp;
X₉ represents Gln, Lys, Ala, Pro, 5,5-dmP, Oic, Oxa, HyP, Aib or Ac5c;
X₁₀ represents Tyr, Phe, 3MePhe, 4MePhe, 4FPhe, 2Nal, 4MeOPhe or 4,4-BPA;
X₁₁ represents Phe, Lys, 4MePhe, 2FPhe, 4FPhe, 4Pal, 4,4-BPA, 4tBuPhe, NO2Phe or 4BrPhe;
X₁₂ represents Ala or Lys;
X₁₃ represents Pro or Lys;
X₁₄ represents Tyr or Lys; and
X₁₅ represents Met, Lys, Nle, HLeu or Ahp,
wherein Chg represents L-cyclohexyl glycine; Aad represents 2-aminoadipic acid; HyP represents hydroxyproline; Oxa represents oxazolidine-4-carboxylic acid; 5,5-dmP represents 5,5-Dimethyl-L-proline; Oic represents octahydroindolecarboxylic acid; Aib represents aminoisobutyric acid;
Ac5c represents aminocyclopentanecarboxylic acid; MePhe represents methyl phenylalanine;
FPhe represents fluorophenylalanine; Nal represents napthylalanine; MeOPhe represents methoxy phenylalanine; 4,4-BPA represents 4,4-biphenylalanine; PaI represents pyridylalanine; tBuPhe represents t-butyl phenylalanine; NO2Phe represents nitro phenylalanine; 4BrPhe represents bromo phenylalanine; HLeu represents homoleucine; and Ahp represents aminoheptanoic acid.

2-16. (canceled)

17. The peptide ligand, or a pharmaceutically acceptable salt thereof, as defined in claim 1, wherein the amino acid sequence of SEQ ID NO: 266 is selected from one of the following peptides, or a modified derivative thereof:
(SEQ ID NO: 268)
CIK(Peg12)EGQYCFADPYMC;
(SEQ ID NO: 269)
CIEK(Peg12)GQYCFADPYMC;
(SEQ ID NO: 270)
CIEEGK(Peg12)YCFADPYMC;
(SEQ ID NO: 271)
CIEEGQYCK(Peg12)ADPYMC;
(SEQ ID NO: 272)
CIEEGQYCFK(Peg12)DPYMC;
(SEQ ID NO: 273)
CIEEGQYCFAK(Peg12)PYMC;
(SEQ ID NO: 274)
CIEEGQYCFADK(Peg12)YMC;
(SEQ ID NO: 275)
CIEEGQYCFADPK(Peg12)MC;
(SEQ ID NO: 276)
CIEEGQYCFADPYK(Peg12)C;
(SEQ ID NO: 277)
CIEE[dK(PEG12Fl)]QYCFADPY[Nle]C;
(SEQ ID NO: 278)
CIEEGAYCFADPY(Nle)C;
(SEQ ID NO: 279)
CIEEaQYCFADPY(Nle)C;
(SEQ ID NO: 280)
C-Chg-EEGQYCFADPY[Nle]C;
(SEQ ID NO: 281)
CIPEGQYCFADPY[Nle]C;
(SEQ ID NO: 282)
CIDEGQYCFADPY[Nle]C;
(SEQ ID NO: 283)
CI-Aad-EGQYCFADPY|Nle]C;
(SEQ ID NO: 284)
CIE-Aad-GQYCFADPY[Nle]C;
(SEQ ID NO: 285)
CIEE-DLys-QYCFADPY[Nle]C;
(SEQ ID NO: 286)
CIEE-DPhe-QYCFADPY[Nle]C;
(SEQ ID NO: 287)
CIEE-DGlu-QYCFADPY[Nle]C;
(SEQ ID NO: 288)
CIEE-DGIn-QYCFADPY[Nle]C;
(SEQ ID NO: 289)
CIEE-DLeu-QYCFADPY[Nle]C;
(SEQ ID NO: 290)
CIEE-DSer-QYCFADPY[Nle]C;
(SEQ ID NO: 291)
CIEEGPYCFADPY[Nle]C;
(SEQ ID NO: 292)
CIEEGQFCFADPY[Nle]C;
(SEQ ID NO: 293)
CIEEGQ-3MeF-CFADPY[Nle]C;
(SEQ ID NO: 294)
CIEEGQ-4MeF-CFADPY[Nle]C;
(SEQ ID NO: 295)
CIEEGQ-4FF-CFADPY[Nle]C;
(SEQ ID NO: 296)
CIEEGQYC-4MeF-ADPY[Nle]C;
(SEQ ID NO: 297)
CIEEGQYC-4FF-ADPY[Nle]C;
(SEQ ID NO: 298)
C[tBuAla]EEGQYCFADPY|Nle]C;
(SEQ ID NO: 299)
CIEEGQYC[2FPhe]ADPY[Nle]C;
(SEQ ID NO: 300)
CIEEGQYCFADPY[HLeu]C;
(SEQ ID NO: 301)
CIEEGQYCFADPY[Ahp]C;
(SEQ ID NO: 302)
CIPE[dF]QYCFADPY[Nle]C;
(SEQ ID NO: 303)
CIPE[dF]PYCFADPY[Nle]C;
(SEQ ID NO: 304)
CIEE[dF]PYCFADPY[Nle]C;
(SEQ ID NO: 305)
CIPEGPYCFADPY[Nle]C;
(SEQ ID NO: 306)
C[tBuAla]PE[dF]P[4MePhe]C[4FPhe]ADPY[Nle]C;
(SEQ ID NO: 307)
C[tBuAla]PE[dF]Q[4MePhe]C[4FPhe]ADPY[Nle]C;
(SEQ ID NO: 308)
C[tBuAla]EE[ dF]P[4MePhe]C[4FPhe]ADPY[Nle]C;
(SEQ ID NO: 309)
CI[HyPJEGQYCFADPY[Nle]C;
(SEQ ID NO: 310)
CIEE[dW]QYCFADPY[Nle]C;
(SEQ ID NO: 311)
CIEEGQ[2Nal]CFADPY[Nle]C;
(SEQ ID NO: 312)
CIEEGQ[4MeoPhe]CFADPY[Nle]C;
(SEQ ID NO: 313)
CIEEGQ[44BPA]CFADPY[Nle]C;
(SEQ ID NO: 314)
CIEEGQYC[4Pal]ADPY[Nle]C;
(SEQ ID NO: 315)
CIEEGQYC[44BPA]ADPY[Nle]C;
(SEQ ID NO: 316)
CIEEGQYC[4tBuPhe]ADPY[Nle]C;
(SEQ ID NO: 317)
CIEEG[55DMP]YCFADPY|Nle]C;
(SEQ ID NO: 318)
CIEEG[Oic]YCFADPY[Nle]C;
(SEQ ID NO: 319)
CI[Oxa]EGQYCFADPY[Nle]C;
(SEQ ID NO: 320)
CIEEG[Oxa]YCFADPY[Nle]C;
(SEQ ID NO: 321)
CIPEGPYCFADPY[Nle]C;
(SEQ ID NO: 322)
CIEEG[HyP]YCFADPY|Nle]C;
(SEQ ID NO: 323)
CIPE[dA]PYCFADPY[Nle]C;
(SEQ ID NO: 324)
C[tBuAla]EEGQYCFADPY[Nle]C;
(SEQ ID NO: 325)
CIPEGQYCFADPY[Nle]C;
(SEQ ID NO: 326)
CIEE[dF]QYCFADPY[Nle]C;
(SEQ ID NO: 327)
CIEEG[Aib]YCFADPY[Nle]C;
(SEQ ID NO: 328)
CIEEG[AC5C]YCFADPY[Nle]C;
(SEQ ID NO: 329)
CIEEGQYC[NO2Phe]ADPY[Nle]C;
(SEQ ID NO: 330)
CIEEGQYC[4BrPhe]ADPY[Nle]C;
(SEQ ID NO: 331)
CIEEGQYCFADPYMC;
(SEQ ID NO: 332)
CIEEGQYCFADPY(Nle)C;
(SEQ ID NO: 333)
CIEEGQYCFADPY[Nle]C; and
and
(SEQ ID NO: 334)
C[tBuAla]PE[dA]PYCFADPY[Nle]C.

18. The peptide ligand, or a pharmaceutically acceptable salt thereof, as defined claim 1, which comprises three cysteine residues separated by two loop sequences both of which consist of 6 amino acids, and said peptide ligand comprises an amino acid sequence which is SEQ ID NO: 267, or a modified derivative thereof:
(SEQ ID NO: 267)
Cᵢ-X₅-X₆-X₇-X₈-X₉-X₁₀-Cᵢᵢ-X₁₁-A-D-P-Y-X₁₅-Cᵢᵢᵢ;
wherein Cᵢ, Cᵢᵢ and Cᵢᵢᵢ represent first, second and third cysteine residues, respectively;
X₅ represents Ile or tBuAla;
X₆ represents Lys, Glu or Pro;
X₇ represents Glu or D-Lys;
X₈ represents Gly, D-Lys, D-Phe or D-Ala;
X₉ represents Gln, Lys or Pro;
X₁₀ represents Tyr or 4MePhe;
X₁₁ represents Phe or 4FPhe; and
X₁₅ represents Met or Nle.

19. The peptide ligand, or a pharmaceutically acceptable salt thereof, as defined in claim 18, wherein the sequence of SEQ ID NO: 267, or a modified derivative thereof, comprises a sequence selected from one of the following, or a modified derivative thereof:
(SEQ ID NO: 335)
CIK(Peg12)EGQYCFADPYMC;
(SEQ ID NO: 336)
CIEK(Peg12)GQYCFADPYMC;
(SEQ ID NO: 337)
CIEEGK(Peg12)YCFADPYMC;
(SEQ ID NO: 338)
CIEE[dK(PEG12Fl)]QYCFADPY[Nle]C;
(SEQ ID NO: 339)
CIPEGQYCFADPY[Nle]C;
(SEQ ID NO: 340)
CIEE-DPhe-QYCFADPY[Nle]C;
(SEQ ID NO: 341)
CIEEGPYCFADPY[Nle]C;
(SEQ ID NO: 342)
C[tBuAla]PE[dF]P[4MePhe]C[4FPhe]ADPY[Nle]C;
(SEQ ID NO: 343)
CIEEGQYCFADPYMC;
(SEQ ID NO: 344)
CIEEGQYCFADPY(Nle)C;
(SEQ ID NO: 345)
CIEEGQYCFADPY[Nle]C; and
and
(SEQ ID NO: 346)
C[tBuAla]PE[dA]PYCFADPY|Nle]C.

20. The peptide ligand, or a pharmaceutically acceptable salt thereof, as defined in claim 1, wherein the molecular scaffold is selected from 1,1′,1″-(1,3,5-triazinane-1,3,5-triyl)triprop-2-en-1-one (TATA).

21. The peptide ligand, or a pharmaceutically acceptable salt thereof, as defined in claim 1, wherein the pharmaceutically acceptable salt is selected from the free acid or the sodium, potassium, calcium, ammonium salt.

22. The peptide ligand, or a pharmaceutically acceptable salt thereof, as defined in claim 1, wherein the CD137 is human CD137.

23. A drug conjugate comprising a peptide ligand, or a pharmaceutically acceptable salt thereof, as defined in claim 1, conjugated to one or more effector and/or functional groups.

24. The drug conjugate comprising a peptide ligand, or a pharmaceutically acceptable salt thereof, as defined in claim 1, conjugated to one or more cytotoxic agents.

25. A pharmaceutical composition which comprises the peptide ligand, or a pharmaceutically acceptable salt thereof, of claim 1, or a drug conjugate comprising such a peptide ligand conjugated to one or more effector and/or functional groups, in combination with one or more pharmaceutically acceptable excipients.

26. A method of treating a disease or disorder mediated by CD137, wherein said method comprises administering the peptide ligand, or a pharmaceutically acceptable salt thereof, as defined in claim 1 or a drug conjugate comprising such a peptide ligand, or a pharmaceutically acceptable salt thereof, conjugated to one or more effector and/or functional groups, to a subject in need thereof.

27. The peptide ligand, or a pharmaceutically acceptable salt thereof, as defined in claim 1, wherein the peptide ligand comprises a sequence selected from one of the following peptides or a modified derivative thereof:
(SEQ ID NO: 52)
Ac-CIK(Peg12)EGQYCFADPYMC;
(SEQ ID NO: 53)
Ac-CIEK(Peg12)GQYCFADPYMC;
(SEQ ID NO: 55)
Ac-CIEEGK(Peg12)YCFADPYMC;
(SEQ ID NO: 57)
Ac-CIEEGQYCK(Peg12)ADPYMC;
(SEQ ID NO: 58)
Ac-CIEEGQYCFK(Peg12)DPYMC;
(SEQ ID NO: 59)
Ac-CIEEGQYCFAK(Peg12)PYMC;
(SEQ ID NO: 60)
Ac-CIEEGQYCFADK(Peg12)YMC;
(SEQ ID NO: 61)
Ac-CIEEGQYCFADPK(Peg12)MC;
(SEQ ID NO: 62)
Ac-CIEEGQYCFADPYK(Peg12)C;
(SEQ ID NO: 63)
[Ac]CIEE[dK(PEG12F1)]QYCFADPY[Nle]C;
(SEQ ID NO: 97)
[PEG3]-ACIEEGAYCFADPY(Nle)CA;
(SEQ ID NO: 106)
[PEG3]-ACIEEaQYCFADPY(Nle)CA;
(SEQ ID NO: 117)
[PEG3]-AC-Chg-EEGQYCFADPY[Nle]CA;
(SEQ ID NO: 119)
[PEG3]-ACIPEGQYCFADPY[Nle]CA;
(SEQ ID NO: 120)
[PEG3]-ACIDEGQYCHAUFY[Nle]CA;
(SEQ ID NO: 121)
[PEG3]-ACI-Aad-EGQYCFADPY[Nle]CA;
(SEQ ID NO: 125)
[PEG3]-ACIE-Aad-GQYCFADPY[Nle]CA;
(SEQ ID NO: 128)
[PEG3]-ACIEE-DLys-QYCFADPY[Nle]CA;
(SEQ ID NO: 129)
[PEG3]-ACIEE-DPhe-QYCFADPY[Nle]CA;
(SEQ ID NO: 130)
[PEG3]-ACIEE-DGlu-QYCFADPY[Nle]CA;
(SEQ ID NO: 131)
[PEG3]-ACIEE-DGln-QYCFADPY[Nle]CA;
(SEQ ID NO: 132)
[PEG3]-ACIEE-DLeu-QYCFADPY[Nle]CA;
(SEQ ID NO: 133)
[PEG3]-ACIEE-DSer-QYCFADPY[Nle]CA;
(SEQ ID NO: 136)
[PEG3]-ACIEEGPYCFADPY[Nle]CA;
(SEQ ID NO: 137)
[PEG3]-ACIEEGQFCFADPY[Nle]CA;
(SEQ ID NO: 139)
[PEG3]-ACIEEGQ-3MeF-CFADPY[Nle]CA;
(SEQ ID NO: 140)
[PEG3]-ACIEEGQ-4MeF-CFADPY[Nle]CA;
(SEQ ID NO: 141)
[PEG3]-ACIEEGQ-4FF-CFADPY[Nle]CA;
(SEQ ID NO: 145)
[PEG3]-ACIEEGQYC-4MeF-ADPY[Nle]CA;
(SEQ ID NO: 147)
[PEG3]-ACIEEGQYC-4FF-ADPY[Nle]CA;
(SEQ ID NO: 156)
[PEG3]AC[tBuAla]EEGQYCFADPY[Nle]CA;
(SEQ ID NO: 159)
[PEG3]ACIEEGQYC[2FPhe]ADPY[Nle]CA;
(SEQ ID NO: 170)
[PEG3]ACIEEGQYCFADPY[HLeu]CA;
(SEQ ID NO: 188)
[PEG3]-ACIEEGQYCFADPY[Ahp]CA;
(SEQ ID NO: 195)
[PEG3]ACIPE[dF]QYCFADPY[Nle]CA;
(SEQ ID NO: 196)
[PEG3]ACIPE[dF]PYCFADPY[Nle]CA;
(SEQ ID NO: 197)
[PEG3]ACIEE[dF]PYCFADPY[Nle]CA;
(SEQ ID NO: 198)
[PEG3]ACIPEGPYCFADPY[Nle]CA;
(SEQ ID NO: 199)
[PEG3]AC[tBuAla]PE[dF]P[4MePhe]C[4FPhe]ADPY[Nle]
CA;
(SEQ ID NO: 200)
[PEG3]AC[tBuAla]PE[dF]Q[4MePhe]C[4FPhe]ADPY[Nle]
CA;
(SEQ ID NO: 201)
[PEG3]AC[tBuAla]EE[dF]P[4MePhe]C[4FPhe]ADPY[Nle]CA;
(SEQ ID NO: 203)
[PEG3]ACI[HyP]EGQYCFADPY[Nle]CA;
(SEQ ID NO: 204)
[PEG3]ACIEE[dW]QYCFADPY[Nle]CA;
(SEQ ID NO: 207)
[PEG3]ACIEEGQ[2Nal]CFADPY[Nle]CA;
(SEQ ID NO: 208)
[PEG3]ACIEEGQ[4MeoPhe]CFADPY[Nle]CA;
(SEQ ID NO: 223)
[Ac]ACIEEGQ[44BPA]CFADPY[Nle]CA;
(SEQ ID NO: 227)
[Ac]ACIEEGQYC[4Pal]ADPY[Nle]CA;
(SEQ ID NO: 228)
[Ac]ACIEEGQYC[44BPA]ADPY[Nle]CA;
(SEQ ID NO: 230)
[Ac]ACIEEGQYC[4tBuPhe]ADPY[Nle]CA;
(SEQ ID NO: 232)
[Ac]ACIEEG[55DMP]YCFADPY[Nle]CA;
(SEQ ID NO: 234)
[Ac]ACIEEG[Oic]YCFADPY[Nle]CA;
(SEQ ID NO: 238)
[Ac]ACI[Oxa]EGQYCFADPY[Nle]CA;
(SEQ ID NO: 239)
[Ac]ACIEEG[Oxa]YCFADPY[Nle]CA;
(SEQ ID NO: 240)
[Ac]ACIPEGPYCFADPY[Nle]CA;
(SEQ ID NO: 241)
[Ac]ACIEEG[HyP]YCFADPY[Nle]CA;
(SEQ ID NO: 242)
[Ac]ACIPE[dA]PYCFADPY[Nle]CA;
(SEQ ID NO: 245)
[Ac]AC[tBuAla]EEGQYCFADPY[Nle]CA;
(SEQ ID NO: 248)
[Ac]ACIPEGQYCFADPY[Nle]CA;
(SEQ ID NO: 252)
[Ac]ACIEE[dF]QYCFADPY[Nle]CA;
(SEQ ID NO: 256)
[Ac]ACIEEG[Aib]YCFADPY[Nle]CA;
(SEQ ID NO: 257)
[Ac]ACIEEG[AC5C]YCFADPY[Nle]CA;
(SEQ ID NO: 261)
[Ac]ACIEEGQYC[NO2Phe]ADPY[Nle]CA;
(SEQ ID NO: 262)
[Ac]ACIEEGQYC[4BrPhe]ADPY[Nle]CA;
(SEQ ID NO: 265)
[Ac]ACIEEGQYCFADPYMCA;
(SEQ ID NO: 31)
ACIEEGQYCFADPY(Nle)CA;
(SEQ ID NO: 194)
[Ac]CIEEGQYCFADPY[Nle]C[Dap];
and
(SEQ ID NO: 243)
[Ac]AC[tBuAla]PE[dA]PYCFADPY[Nle]CA.

28. The peptide ligand, or a pharmaceutically acceptable salt thereof, as defined in claim 1, wherein said peptide ligand comprises an amino acid sequence which is SEQ ID NO: 267, or a modified derivative thereof:
(SEQ ID NO: 267)
Cᵢ-X₅-X₆-X₇-X₈-X₉-X₁₀-Cᵢᵢ-X₁₁-A-D-P-Y-X₁₅-Cᵢᵢᵢ;
wherein Cᵢ, Cᵢᵢ and Cᵢᵢᵢ represent first, second and third cysteine residues, respectively;
X₅ represents tBuAla;
X₆ represents Pro;
X₇ represents Glu;
X₈ represents D-Lys;
X₉ represents Pro;
X₁₀ represents Tyr;
X₁₁ represents Phe; and
X₁₅ represents Nle.

29. The peptide ligand, or a pharmaceutically acceptable salt thereof, as defined in claim 1, wherein the peptide ligand comprises a sequence selected from one of the following peptides, or modified derivatives thereof:
(SEQ ID NO: 52)
Ac-CIK(Peg12)EGQYCFADPYMC;
(SEQ ID NO: 53)
Ac-CIEK(Peg12)GQYCFADPYMC;
(SEQ ID NO: 55)
Ac-CIEEGK(Peg12)YCFADPYMC;
(SEQ ID NO: 63)
[Ac]CIEE[dK(PEG12F1)]QYCFADPY[Nle]C;
(SEQ ID NO: 119)
[PEG3]-ACIPEGQYCFADPY[Nle]CA;
(SEQ ID NO: 129)
[PEG3]-ACIEE-DPhe-QYCFADPY[Nle]CA;
(SEQ ID NO: 136)
[PEG3]-ACIEEGPYCFADPY[Nle]CA;
(SEQ ID NO: 199)
[PEG3]AC[tBuAla]PE[dF]P[4MePhe]C[4FPhe]ADPY[Nle]
CA;
(SEQ ID NO: 265)
[Ac]ACIEEGQYCFADPYMCA;
(SEQ ID NO: 31)
ACIEEGQYCFADPY(Nle)CA;
(SEQ ID NO: 194)
[Ac]CIEEGQYCFADPY[Nle]C[Dap];
and
(SEQ ID NO: 243)
[Ac]AC[tBuAla]PE[dA]PYCFADPY[Nle]CA.

30. The peptide ligand, or a pharmaceutically acceptable salt thereof, as defined in claim 1, wherein the peptide ligand, or a modified derivative thereof, comprises one or more modifications selected from:
replacement of one or more amino acid residues with one or more non-natural amino acid residues;
replacement of one or more polar amino acid residues with one or more isosteric or isoelectronic amino acids;
replacement of one or more non-polar amino acid residues with other non-natural isosteric or isoelectronic amino acids;
replacement of one or more oxidation sensitive amino acid residues with one or more oxidation resistant amino acid residues;
replacement of one or more L-amino acid residues with one or more D-amino acid residues;
N-alkylation of one or more amide bonds within the bicyclic peptide ligand;
replacement of one or more peptide bonds with a surrogate bond;
substitution of the hydrogen on the alpha-carbon of one or more amino acid residues with another chemical group; and
introduction or replacement of amino acids that introduce orthogonal reactivities that are suitable for functionalisation.

31. The peptide ligand, or a pharmaceutically acceptable salt thereof, as defined in claim 30, wherein the peptide ligand, or a modified derivative thereof, comprises one or more modifications, wherein said modification comprises introduction or replacement of amino acids that introduce orthogonal reactivities that are suitable for functionalisation.

32. The peptide ligand, or a pharmaceutically acceptable salt thereof, as defined in claim 31, wherein the peptide ligand, or a modified derivative thereof, comprises one or more modifications, wherein said modification comprises introduction of an azide or alkyne-group bearing amino acid.


SEQUENCE LISTING (346 sequences)

SEQ ID NO: 1 - protein, 15 aa, synthetic construct
  REGION 1..15: note = Synthetic Peptide
CIEEGQYCYRDMYMC

SEQ ID NO: 2 - protein, 15 aa, synthetic construct
  REGION 1..15: note = Synthetic Peptide
CIEEGQYCYADPYMC

SEQ ID NO: 3 - protein, 15 aa, synthetic construct
  REGION 1..15: note = Synthetic Peptide
CIEEGQYCYADPYYC

SEQ ID NO: 4 - protein, 15 aa, synthetic construct
  REGION 1..15: note = Synthetic Peptide
CIEEGQYCYSDPYYC

SEQ ID NO: 5 - protein, 15 aa, synthetic construct
  REGION 1..15: note = Synthetic Peptide
CIEEGQYCFADPYMC

SEQ ID NO: 6 - protein, 15 aa, synthetic construct
  REGION 1..15: note = Synthetic Peptide
CIEEGQYCYADHQLC

SEQ ID NO: 7 - protein, 15 aa, synthetic construct
  REGION 1..15: note = Synthetic Peptide
CIEEGQYCHADPYYC

SEQ ID NO: 8 - protein, 15 aa, synthetic construct
  REGION 1..15: note = Synthetic Peptide
CIEEGQYCHADPYFC

SEQ ID NO: 9 - protein, 15 aa, synthetic construct
  REGION 1..15: note = Synthetic Peptide
CIEEGQYCYADHYMC

SEQ ID NO: 10 - protein, 15 aa, synthetic construct
  REGION 1..15: note = Synthetic Peptide
CIEEGQYCYADPYLC

SEQ ID NO: 11 - protein, 15 aa, synthetic construct
  REGION 1..15: note = Synthetic Peptide
CIEEGQYCYSDPYLC

SEQ ID NO: 12 - protein, 15 aa, synthetic construct
  REGION 1..15: note = Synthetic Peptide
CIEEGQYCFADPYLC

SEQ ID NO: 13 - protein, 15 aa, synthetic construct
  REGION 1..15: note = Synthetic Peptide
CIEEGQYCHADPYMC

SEQ ID NO: 14 - protein, 15 aa, synthetic construct
  REGION 1..15: note = Synthetic Peptide
CIEEGQYCHADPQMC

SEQ ID NO: 15 - protein, 15 aa, synthetic construct
  REGION 1..15: note = Synthetic Peptide
CDIGPPYCYRDMYMC

SEQ ID NO: 16 - protein, 15 aa, synthetic construct
  REGION 1..15: note = Synthetic Peptide
CDIGPPYCYADPYMC

SEQ ID NO: 17 - protein, 14 aa, synthetic construct
  REGION 1..14: note = Synthetic Peptide
CDEWGLFCIPHSDC

SEQ ID NO: 18 - protein, 14 aa, synthetic construct
  REGION 1..14: note = Synthetic Peptide
CDEWGLYCFAHPDC

SEQ ID NO: 19 - protein, 15 aa, synthetic construct
  REGION 1..15: note = Synthetic Peptide
CIEPGPFCYADPYMC

SEQ ID NO: 20 - protein, 15 aa, synthetic construct
  REGION 1..15: note = Synthetic Peptide
  SITE 9..10: note = misc_feature - Xaa can be any naturally occurring amino acid
  SITE 12: note = misc_feature - Xaa can be any naturally occurring amino acid
  SITE 13: note = X - X represents Y, Q or M
  SITE 14: note = misc_feature - Xaa can be any naturally occurring amino acid
CIEEGQYCXXDXXXC

SEQ ID NO: 21 - protein, 15 aa, synthetic construct
  REGION 1..15: note = Synthetic Peptide
  SITE 10: note = X - X represents R or A
  SITE 12: note = X - X represents M or P
CDIGPPYCYXDXYMC

SEQ ID NO: 22 - protein, 14 aa, synthetic construct
  REGION 1..14: note = Synthetic Peptide
  SITE 7: note = X - X represents F or Y
  SITE 9: note = X - X represents I or F
  SITE 10: note = X - X represents P or A
  SITE 12: note = X - X represents S or P
CDEWGLXCXXHXDC

SEQ ID NO: 23 - protein, 15 aa, synthetic construct
  REGION 1..15: note = Synthetic Peptide
  SITE 14: note = X - X represent Nle
CIEEGQYCFADPYXC

SEQ ID NO: 24 - protein, 15 aa, synthetic construct
  REGION 1..15: note = Synthetic Peptide
  SITE 14: note = X - X represents Nle
CIKEGQYCFADPYXC

SEQ ID NO: 25 - protein, 15 aa, synthetic construct
  REGION 1..15: note = Synthetic Peptide
  SITE 14: note = X - X represents Nle
CIEKGQYCFADPYXC

SEQ ID NO: 26 - protein, 15 aa, synthetic construct
  REGION 1..15: note = Synthetic Peptide
  SITE 14: note = X - X represents Nle
  SITE 5: note = D-Lysine
CIEEKQYCFADPYXC

SEQ ID NO: 27 - protein, 15 aa, synthetic construct
  REGION 1..15: note = Synthetic Peptide
  SITE 14: note = X - X represents Nle
CIEEGKYCFADPYXC

SEQ ID NO: 28 - protein, 15 aa, synthetic construct
  REGION 1..15: note = Synthetic Peptide
  SITE 14: note = X - X represents Nle
CIEEGQYCKADPYXC

SEQ ID NO: 29 - protein, 15 aa, synthetic construct
  REGION 1..15: note = Synthetic Peptide
  SITE 14: note = X - X represents Nle
CIEEGQYCFADKYXC

SEQ ID NO: 30 - protein, 15 aa, synthetic construct
  REGION 1..15: note = Synthetic Peptide
CIEEGQYCFADPYKC

SEQ ID NO: 31 - protein, 17 aa, synthetic construct
  REGION 1..17: note = Synthetic Peptide
  SITE 15: note = X - X represents Nle
ACIEEGQYCFADPYXCA

SEQ ID NO: 32 - protein, 16 aa, synthetic construct
  REGION 1..16: note = Synthetic Peptide
  SITE 15: note = X - X represents Nle
  SITE 1: note = Acetylation of the N-terminus
  SITE 16: note = diaminopropionic acid modification on C terminus
ACIEEGQYCFADPYXC

SEQ ID NO: 33 - protein, 17 aa, synthetic construct
  REGION 1..17: note = Synthetic Peptide
  SITE 15: note = X - X represents Nle
  SITE 1: note = Acetylation of the N-terminus
ACIKEGQYCFADPYXCA

SEQ ID NO: 34 - protein, 17 aa, synthetic construct
  REGION 1..17: note = Synthetic Peptide
  SITE 15: note = X - X represents Nle
  SITE 1: note = Acetylation of the N-terminus
ACIEKGQYCFADPYXCA

SEQ ID NO: 35 - protein, 17 aa, synthetic construct
  REGION 1..17: note = Synthetic Peptide
  SITE 15: note = X - X represents Nle
  SITE 1: note = Acetylation of the N-terminus
  SITE 6: note = D-Lysine
ACIEEKQYCFADPYXCA

SEQ ID NO: 36 - protein, 17 aa, synthetic construct
  REGION 1..17: note = Synthetic Peptide
  SITE 15: note = X - X represents Nle
  SITE 1: note = Acetylation of the N-terminus
ACIEEGKYCFADPYXCA

SEQ ID NO: 37 - protein, 17 aa, synthetic construct
  REGION 1..17: note = Synthetic Peptide
  SITE 15: note = X - X represents Nle
  SITE 1: note = Acetylation of the N-terminus
ACIEEGQYCKADPYXCA

SEQ ID NO: 38 - protein, 17 aa, synthetic construct
  REGION 1..17: note = Synthetic Peptide
  SITE 15: note = X - X represents Nle
  SITE 1: note = Acetylation of the N-terminus
ACIEEGQYCFADKYXCA

SEQ ID NO: 39 - protein, 17 aa, synthetic construct
  REGION 1..17: note = Synthetic Peptide
  SITE 1: note = Acetylation of the N-terminus
ACIEEGQYCFADPYKCA

SEQ ID NO: 40 - protein, 15 aa, synthetic construct
  REGION 1..15: note = Synthetic Peptide
CLPPGQYCFPDLLLC

SEQ ID NO: 41 - protein, 15 aa, synthetic construct
  REGION 1..15: note = Synthetic Peptide
  SITE 2: note = X - X represents I, L, M or V
  SITE 3: note = X - X represents E, D, P or S
  SITE 4: note = X - X represents P, E or A
  SITE 6: note = X - X represents P or Q
  SITE 7: note = X - X represents Y or F
  SITE 13: note = X - X represents Y or M
  SITE 14: note = X - X represents M, L or Y
CXXXGXXCYADPXXC

SEQ ID NO: 42 - protein, 23 aa, synthetic construct
  REGION 1..17: note = Synthetic Peptide
  SITE 1: note = B-alanine
  SITE 2..6: note = Sarcosine
AXXXXXACIEEGQYCYRDMYMCA

SEQ ID NO: 43 - protein, 24 aa, synthetic construct
  REGION 1..17: note = Synthetic Peptide
  SITE 1: note = Acetylation of the N-terminus
  SITE 18..23: note = Sarcosine
  MOD_RES 24: note = Fl side chain modification
ACIEEGQYCYRDMYMCAXXXXXXK

SEQ ID NO: 44 - protein, 24 aa, synthetic construct
  REGION 1..17: note = Synthetic Peptide
  SITE 18..23: note = Sarcosine
  SITE 24: note = Fl side chain modification
ACIEEGQYCYADPYMCAXXXXXXK

SEQ ID NO: 45 - protein, 23 aa, synthetic construct
  REGION 1..17: note = Synthetic Peptide
  SITE 1: note = B-alanine
  SITE 2..6: note = Sarcosine
AXXXXXACIEEGQYCYADPYMCA

SEQ ID NO: 46 - protein, 24 aa, synthetic construct
  REGION 1..17: note = Synthetic Peptide
  SITE 18..23: note = Sarcosine
ACIEEGQYCYADPYYCAXXXXXXK

SEQ ID NO: 47 - protein, 23 aa, synthetic construct
  REGION 1..17: note = Synthetic Peptide
  SITE 1: note = B-alanine
  SITE 2..6: note = Sarcosine
AXXXXXACIEEGQYCYADPYYCA

SEQ ID NO: 48 - protein, 24 aa, synthetic construct
  REGION 1..17: note = Synthetic Peptide
  SITE 18..23: note = Sarcosine
ACIEEGQYCYSDPYYCAXXXXXXK

SEQ ID NO: 49 - protein, 24 aa, synthetic construct
  REGION 1..17: note = Synthetic Peptide
  SITE 18..23: note = Sarcosine
ACIEEGQYCFADPYMCAXXXXXXK

SEQ ID NO: 50 - protein, 23 aa, synthetic construct
  REGION 1..17: note = Synthetic Peptide
  SITE 2..6: note = Sarcosine
GXXXXXACIEEGQYCFADPYMCA

SEQ ID NO: 51 - protein, 15 aa, synthetic construct
  REGION 1..15: note = Synthetic Peptide
  SITE 1: note = Acetylation of the N-terminus
  MOD_RES 2: note = PEG12 side chain modification
CKEEGQYCFADPYMC

SEQ ID NO: 52 - protein, 15 aa, synthetic construct
  REGION 1..15: note = Synthetic Peptide
  SITE 1: note = Acetylation of the N-terminus
  MOD_RES 3: note = PEG12 side chain modification
CIKEGQYCFADPYMC

SEQ ID NO: 53 - protein, 15 aa, synthetic construct
  REGION 1..15: note = Synthetic Peptide
  SITE 1: note = Acetylation of the N-terminus
  MOD_RES 4: note = PEG12 side chain modification
CIEKGQYCFADPYMC

SEQ ID NO: 54 - protein, 15 aa, synthetic construct
  REGION 1..15: note = Synthetic Peptide
  SITE 1: note = Acetylation of the N-terminus
  MOD_RES 5: note = PEG12 side chain modification
CIEEKQYCFADPYMC

SEQ ID NO: 55 - protein, 15 aa, synthetic construct
  REGION 1..15: note = Synthetic Peptide
  SITE 1: note = Acetylation of the N-terminus
  MOD_RES 6: note = PEG12 side chain modification
CIEEGKYCFADPYMC

SEQ ID NO: 56 - protein, 15 aa, synthetic construct
  REGION 1..15: note = Synthetic Peptide
  SITE 1: note = Acetylation of the N-terminus
  MOD_RES 7: note = PEG12 side chain modification
CIEEGQKCFADPYMC

SEQ ID NO: 57 - protein, 15 aa, synthetic construct
  REGION 1..15: note = Synthetic Peptide
  SITE 1: note = Acetylation of the N-terminus
  MOD_RES 9: note = PEG12 side chain modification
CIEEGQYCKADPYMC

SEQ ID NO: 58 - protein, 15 aa, synthetic construct
  REGION 1..15: note = Synthetic Peptide
  SITE 1: note = Acetylation of the N-terminus
  MOD_RES 10: note = PEG12 side chain modification
CIEEGQYCFKDPYMC

SEQ ID NO: 59 - protein, 15 aa, synthetic construct
  REGION 1..15: note = Synthetic Peptide
  SITE 1: note = Acetylation of the N-terminus
  MOD_RES 11: note = PEG12 side chain modification
CIEEGQYCFAKPYMC

SEQ ID NO: 60 - protein, 15 aa, synthetic construct
  REGION 1..15: note = Synthetic Peptide
  SITE 1: note = Acetylation of the N-terminus
  MOD_RES 12: note = PEG12 side chain modification
CIEEGQYCFADKYMC

SEQ ID NO: 61 - protein, 15 aa, synthetic construct
  REGION 1..15: note = Synthetic Peptide
  SITE 1: note = Acetylation of the N-terminus
  MOD_RES 13: note = PEG12 side chain modification
CIEEGQYCFADPKMC

SEQ ID NO: 62 - protein, 15 aa, synthetic construct
  REGION 1..15: note = Synthetic Peptide
  SITE 1: note = Acetylation of the N-terminus
  MOD_RES 14: note = PEG12 side chain modification
CIEEGQYCFADPYKC

SEQ ID NO: 63 - protein, 15 aa, synthetic construct
  REGION 1..15: note = Synthetic Peptide
  SITE 14: note = X - X represents Nle
  SITE 1: note = Acetylation of the N-terminus
  MOD_RES 5: note = PEG12Fl side chain modification
  MOD_RES 5: note = D-lysine
CIEEKQYCFADPYXC

SEQ ID NO: 64 - protein, 17 aa, synthetic construct
  REGION 1..17: note = Synthetic Peptide
  SITE 15: note = X - X represents Nle
ACIEEGQYCFADPYXCA

SEQ ID NO: 65 - protein, 17 aa, synthetic construct
  REGION 1..17: note = Synthetic Peptide
  SITE 15: note = X - X represents Nle
  MOD_RES 1: note = PEG12Fl side chain modification
ACIEEGQYCFADPYXCA

SEQ ID NO: 66 - protein, 24 aa, synthetic construct
  REGION 1..17: note = Synthetic Peptide
  SITE 18..23: note = Sarcosine
ACIEEGQYCYADHQLCAXXXXXXK

SEQ ID NO: 67 - protein, 24 aa, synthetic construct
  REGION 1..17: note = Synthetic Peptide
  SITE 18..23: note = Sarcosine
ACIEEGQYCHADPYYCAXXXXXXK

SEQ ID NO: 68 - protein, 24 aa, synthetic construct
  REGION 1..17: note = Synthetic Peptide
  SITE 18..23: note = Sarcosine
ACIEEGQYCHADPYFCAXXXXXXK

SEQ ID NO: 69 - protein, 24 aa, synthetic construct
  REGION 1..17: note = Synthetic Peptide
  SITE 18..23: note = Sarcosine
ACIEEGQYCYADHYMCAXXXXXXK

SEQ ID NO: 70 - protein, 24 aa, synthetic construct
  REGION 1..17: note = Synthetic Peptide
  SITE 18..23: note = Sarcosine
ACIEEGQYCYADPYLCAXXXXXXK

SEQ ID NO: 71 - protein, 26 aa, synthetic construct
  REGION 1..19: note = Synthetic Peptide
  SITE 19..25: note = Sarcosine
ACIEEGQYCYADPYLCSVGXXXXXXK

SEQ ID NO: 72 - protein, 25 aa, synthetic construct
  REGION 1..19: note = Synthetic Peptide
  MOD_RES 1: note = Fl side chain modification
  SITE 2..6: note = Sarcosine
GXXXXXACIEEGQYCYADPYLCSVG

SEQ ID NO: 73 - protein, 24 aa, synthetic construct
  REGION 1..17: note = Synthetic Peptide
  SITE 18..23: note = Sarcosine
ACIEEGQYCYSDPYLCAXXXXXXK

SEQ ID NO: 74 - protein, 24 aa, synthetic construct
  REGION 1..17: note = Synthetic Peptide
  SITE 18..23: note = Sarcosine
ACIEEGQYCFADPYLCAXXXXXXK

SEQ ID NO: 75 - protein, 24 aa, synthetic construct
  REGION 1..17: note = Synthetic Peptide
  SITE 18..23: note = Sarcosine
ACIEEGQYCHADPYMCAXXXXXXK

SEQ ID NO: 76 - protein, 24 aa, synthetic construct
  REGION 1..17: note = Synthetic Peptide
  SITE 18..23: note = Sarcosine
ACIEEGQYCHADPQMCAXXXXXXK

SEQ ID NO: 77 - protein, 24 aa, synthetic construct
  REGION 1..17: note = Synthetic Peptide
  SITE 18..23: note = Sarcosine
ACDIGPPYCYRDMYMCAXXXXXXK

SEQ ID NO: 78 - protein, 23 aa, synthetic construct
  REGION 1..16: note = Synthetic Peptide
  SITE 17..22: note = Sarcosine
ADIGPPYCYADPYMCAXXXXXXK

SEQ ID NO: 79 - protein, 24 aa, synthetic construct
  REGION 1..17: note = Synthetic Peptide
  SITE 18..23: note = Sarcosine
ACLDPGPFCFADPYMCAXXXXXXK

SEQ ID NO: 80 - protein, 24 aa, synthetic construct
  REGION 1..17: note = Synthetic Peptide
  SITE 18..23: note = Sarcosine
ACLDEGPYCFADPYFCAXXXXXXK

SEQ ID NO: 81 - protein, 24 aa, synthetic construct
  REGION 1..17: note = Synthetic Peptide
  SITE 18..23: note = Sarcosine
ACINEGPYCFADPYMCAXXXXXXK

SEQ ID NO: 82 - protein, 24 aa, synthetic construct
  REGION 1..17: note = Synthetic Peptide
  SITE 18..23: note = Sarcosine
ACIEQGPFCFADPYMCAXXXXXXK

SEQ ID NO: 83 - protein, 24 aa, synthetic construct
  REGION 1..17: note = Synthetic Peptide
  SITE 18..23: note = Sarcosine
ACVEEGPFCFADPYYCAXXXXXXK

SEQ ID NO: 84 - protein, 24 aa, synthetic construct
  REGION 1..17: note = Synthetic Peptide
  SITE 18..23: note = Sarcosine
ACLDEGPFCFSDPYMCAXXXXXXK

SEQ ID NO: 85 - protein, 22 aa, synthetic construct
  REGION 1..16: note = Synthetic Peptide
  SITE 1: note = B-alanine
  SITE 2..6: note = Sarcosine
AXXXXXACDEWGLFCIPHSDCA

SEQ ID NO: 86 - protein, 23 aa, synthetic construct
  REGION 1..16: note = Synthetic Peptide
  SITE 17..22: note = Sarcosine
ACDEWGLYCFAHPDCAXXXXXXK

SEQ ID NO: 87 - protein, 26 aa, synthetic construct
  REGION 1..19: note = Synthetic Peptide
  SITE 20..25: note = Sarcosine
ACLDPGPYCYADPYMCTFHXXXXXXK

SEQ ID NO: 88 - protein, 26 aa, synthetic construct
  REGION 1..19: note = Synthetic Peptide
  SITE 20..25: note = Sarcosine
ACIEPGPFCYADPYMCNRVXXXXXXK

SEQ ID NO: 89 - protein, 25 aa, synthetic construct
  REGION 1..19: note = Synthetic Peptide
  SITE 2..6: note = Sarcosine
GXXXXXACIEPGPFCYADPYMCNRV

SEQ ID NO: 90 - protein, 26 aa, synthetic construct
  REGION 1..19: note = Synthetic Peptide
  SITE 20..25: note = Sarcosine
ACLEPGPYCYADPYMCTHLXXXXXXK

SEQ ID NO: 91 - protein, 24 aa, synthetic construct
  REGION 1..17: note = Synthetic Peptide
  SITE 18..23: note = Sarcosine
ACLPPGPYCFPDPYFCAXXXXXXK

SEQ ID NO: 92 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADPYXCA

SEQ ID NO: 93 - protein, 17 aa, synthetic construct
  REGION 1..17: note = Synthetic Peptide
  SITE 15: note = X - X represents Nle
  MOD_RES 1: note = PEG3 side chain modification
ACAEEGQYCFADPYXCA

SEQ ID NO: 94 - protein, 17 aa, synthetic construct
  REGION 1..17: note = Synthetic Peptide
  SITE 15: note = X - X represents Nle
  MOD_RES 1: note = PEG3 side chain modification
ACIAEGQYCFADPYXCA

SEQ ID NO: 95 - protein, 17 aa, synthetic construct
  REGION 1..17: note = Synthetic Peptide
  SITE 15: note = X - X represents Nle
  MOD_RES 1: note = PEG3 side chain modification
ACIEAGQYCFADPYXCA

SEQ ID NO: 96 - protein, 17 aa, synthetic construct
  REGION 1..17: note = Synthetic Peptide
  SITE 15: note = X - X represents Nle
  MOD_RES 1: note = PEG3 side chain modification
ACIEEAQYCFADPYXCA

SEQ ID NO: 97 - protein, 17 aa, synthetic construct
  REGION 1..17: note = Synthetic Peptide
  SITE 15: note = X - X represents Nle
  MOD_RES 1: note = PEG3 side chain modification
ACIEEGAYCFADPYXCA

SEQ ID NO: 98 - protein, 17 aa, synthetic construct
  REGION 1..17: note = Synthetic Peptide
  SITE 15: note = X - X represents Nle
  MOD_RES 1: note = PEG3 side chain modification
ACIEEGQACFADPYXCA

SEQ ID NO: 99 - protein, 17 aa, synthetic construct
  REGION 1..17: note = Synthetic Peptide
  SITE 15: note = X - X represents Nle
  MOD_RES 1: note = PEG3 side chain modification
ACIEEGQYCAADPYXCA

SEQ ID NO: 100 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFAAPYXCA

SEQ ID NO: 101 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADPAXCA

SEQ ID NO: 102 - protein, 17 aa, synthetic construct
  REGION 1..17: note = Synthetic Peptide
  MOD_RES 1: note = PEG3 side chain modification
ACIEEGQYCFADPYACA

SEQ ID NO: 103 - protein, 17 aa, synthetic construct
  REGION 1..17: note = Synthetic Peptide
  SITE 15: note = X - X represents Nle
  MOD_RES 1: note = PEG3 side chain modification
ACAEEGQYCFADPYXCA

SEQ ID NO: 104 - protein, 17 aa, synthetic construct
  REGION 1..17: note = Synthetic Peptide
  SITE 15: note = X - X represents Nle
  MOD_RES 1: note = PEG3 side chain modification
ACIAEGQYCFADPYXCA

SEQ ID NO: 105 - protein, 17 aa, synthetic construct
  REGION 1..17: note = Synthetic Peptide
  SITE 15: note = X - X represents Nle
  MOD_RES 1: note = PEG3 side chain modification
ACIEAGQYCFADPYXCA

SEQ ID NO: 106 - protein, 17 aa, synthetic construct
  REGION 1..17: note = Synthetic Peptide
  SITE 15: note = X - X represents Nle
  MOD_RES 1: note = PEG3 side chain modification
ACIEEAQYCFADPYXCA

SEQ ID NO: 107 - protein, 17 aa, synthetic construct
  REGION 1..17: note = Synthetic Peptide
  SITE 15: note = X - X represents Nle
  MOD_RES 1: note = PEG3 side chain modification
ACIEEGAYCFADPYXCA

SEQ ID NO: 108 - protein, 17 aa, synthetic construct
  REGION 1..17: note = Synthetic Peptide
  SITE 15: note = X - X represents Nle
  MOD_RES 1: note = PEG3 side chain modification
ACIEEGQACFADPYXCA

SEQ ID NO: 109 - protein, 17 aa, synthetic construct
  REGION 1..17: note = Synthetic Peptide
  SITE 15: note = X - X represents Nle
  MOD_RES 1: note = PEG3 side chain modification
ACIEEGQYCAADPYXCA

SEQ ID NO: 110 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADPYXCA

SEQ ID NO: 111 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFAAPYXCA

SEQ ID NO: 112 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADAYXCA

SEQ ID NO: 113 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADPAXCA

SEQ ID NO: 114 - protein, 17 aa, synthetic construct
  REGION 1..17: note = Synthetic Peptide
  MOD_RES 1: note = PEG3 side chain modification
ACIEEGQYCFADPYACA

SEQ ID NO: 115 - protein, 17 aa, synthetic construct
  REGION 1..17: note = Synthetic Peptide
  SITE 15: note = X - X represents Nle
  MOD_RES 1: note = PEG3 side chain modification
ACLEEGQYCFADPYXCA

SEQ ID NO: 116 - protein, 17 aa, synthetic construct
  REGION 1..17: note = Synthetic Peptide
  SITE 3: note = X - X represents Nle
  MOD_RES 1: note = PEG3 side chain modification
  SITE 15: note = X - X represents Nle
ACXEEGQYCFADPYXCA

SEQ ID NO: 117 - protein, 17 aa, synthetic construct
  REGION 1..17: note = Synthetic Peptide
  SITE 3: note = X - X represents Cyclohexylglycine
  SITE 15: note = X - X represents Nle
  MOD_RES 1: note = PEG3 side chain modification
ACXEEGQYCFADPYXCA

SEQ ID NO: 118 - protein, 17 aa, synthetic construct
  REGION 1..17: note = Synthetic Peptide
  SITE 3: note = X - X represents Cyclohexylalanine
  SITE 15: note = X - X represents Nle
  MOD_RES 1: note = PEG3 side chain modification
ACXEEGQYCFADPYXCA

SEQ ID NO: 119 - protein, 17 aa, synthetic construct
  REGION 1..17: note = Synthetic Peptide
  SITE 15: note = X - X represents Nle
  MOD_RES 1: note = PEG3 side chain modification
ACIPEGQYCFADPYXCA

SEQ ID NO: 120 - protein, 17 aa, synthetic construct
  REGION 1..17: note = Synthetic Peptide
  SITE 15: note = X - X represents Nle
  MOD_RES 1: note = PEG3 side chain modification
ACIDEGQYCFADPYXCA

SEQ ID NO: 121 - protein, 17 aa, synthetic construct
  REGION 1..17: note = Synthetic Peptide
  SITE 4: note = X - X represents Aad
  SITE 15: note = X - X represents Nle
  MOD_RES 1: note = PEG3 side chain modification
ACIXEGQYCFADPYXCA

SEQ ID NO: 122 - protein, 17 aa, synthetic construct
  REGION 1..17: note = Synthetic Peptide
  SITE 4: note = X - X represents Amino pimelic acid
  SITE 15: note = X - X represents Nle
  MOD_RES 1: note = PEG3 side chain modification
ACIXEGQYCFADPYXCA

SEQ ID NO: 123 - protein, 17 aa, synthetic construct
  REGION 1..17: note = Synthetic Peptide
  SITE 15: note = X - X represents Nle
  MOD_RES 1: note = PEG3 side chain modification
ACIEPGQYCFADPYXCA

SEQ ID NO: 124 - protein, 17 aa, synthetic construct
  REGION 1..17: note = Synthetic Peptide
  SITE 15: note = X - X represents Nle
  MOD_RES 1: note = PEG3 side chain modification
ACIEDGQYCFADPYXCA

SEQ ID NO: 125 - protein, 17 aa, synthetic construct
  REGION 1..17: note = Synthetic Peptide
  SITE 5: note = X - X represents Aad
  SITE 15: note = X - X represents Nle
  MOD_RES 1: note = PEG3 side chain modification
ACIEXGQYCFADPYXCA

SEQ ID NO: 126 - protein, 17 aa, synthetic construct
  REGION 1..17: note = Synthetic Peptide
  SITE 5: note = X - X represents Amino pimelic acid
  SITE 15: note = X - X represents Nle
  MOD_RES 1: note = PEG3 side chain modification
ACIEXGQYCFADPYXCA

SEQ ID NO: 127 - protein, 17 aa, synthetic construct
  REGION 1..17: note = Synthetic Peptide
  SITE 6: note = X - X represents sarcosine
  SITE 15: note = X - X represents Nle
  MOD_RES 1: note = PEG3 side chain modification
ACIEEXQYCFADPYXCA

SEQ ID NO: 128 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-lysine
ACIEEKQYCFADPYXCA

SEQ ID NO: 129 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-phenylalanine
ACIEEFQYCFADPYXCA

SEQ ID NO: 130 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-glutamic acid
ACIEEEQYCFADPYXCA

SEQ ID NO: 131 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-glutamine
ACIEEQQYCFADPYXCA

SEQ ID NO: 132 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-leucine
ACIEELQYCFADPYXCA

SEQ ID NO: 133 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-serine
ACIEESQYCFADPYXCA

SEQ ID NO: 134 - protein, 17 aa, synthetic construct
  REGION 1..17: note = Synthetic Peptide
  SITE 6: note = X - X represents MeD-Ala
  SITE 15: note = X - X represents Nle
  MOD_RES 1: note = PEG3 side chain modification
ACIEEXQYCFADPYXCA

SEQ ID NO: 135 - protein, 17 aa, synthetic construct
  REGION 1..17: note = Synthetic Peptide
  SITE 6: note = X - X represents Aib
  SITE 15: note = X - X represents Nle
  MOD_RES 1: note = PEG3 side chain modification
ACIEEXQYCFADPYXCA

SEQ ID NO: 136 - protein, 17 aa, synthetic construct
  REGION 1..17: note = Synthetic Peptide
  SITE 15: note = X - X represents Nle
  MOD_RES 1: note = PEG3 side chain modification
ACIEEGPYCFADPYXCA

SEQ ID NO: 137 - protein, 17 aa, synthetic construct
  REGION 1..17: note = Synthetic Peptide
  SITE 15: note = X - X represents Nle
  MOD_RES 1: note = PEG3 side chain modification
ACIEEGQFCFADPYXCA

SEQ ID NO: 138 - protein, 17 aa, synthetic construct
  REGION 1..17: note = Synthetic Peptide
  SITE 8: note = X - X represents 2-methyl phenylalanine
  SITE 15: note = X - X represents Nle
  MOD_RES 1: note = PEG3 side chain modification
ACIEEGQXCFADPYXCA

SEQ ID NO: 139 - protein, 17 aa, synthetic construct
  REGION 1..17: note = Synthetic Peptide
  SITE 8: note = X - X represents 3-methyl phenylalanine
  SITE 15: note = X - X represents Nle
  MOD_RES 1: note = PEG3 side chain modification
ACIEEGQXCFADPYXCA

SEQ ID NO: 140 - protein, 17 aa, synthetic construct
  REGION 1..17: note = Synthetic Peptide
  SITE 8: note = X - X represents 4-methyl phenylalanine
  SITE 15: note = X - X represents Nle
  MOD_RES 1: note = PEG3 side chain modification
ACIEEGQXCFADPYXCA

SEQ ID NO: 141 - protein, 17 aa, synthetic construct
  REGION 1..17: note = Synthetic Peptide
  SITE 8: note = X - X represents fluorophenylalanine
  SITE 15: note = X - X represents Nle
  MOD_RES 1: note = PEG3 side chain modification
ACIEEGQXCFADPYXCA

SEQ ID NO: 142 - protein, 17 aa, synthetic construct
  REGION 1..17: note = Synthetic Peptide
  SITE 8: note = X - X represents fluorophenylalanine
  SITE 15: note = X - X represents Nle
  MOD_RES 1: note = PEG3 side chain modification
ACIEEGQXCFADPYXCA

SEQ ID NO: 143 - protein, 17 aa, synthetic construct
  REGION 1..17: note = Synthetic Peptide
  SITE 10: note = X - X represents methyl phenylalanine
  SITE 15: note = X - X represents Nle
  MOD_RES 1: note = PEG3 side chain modification
ACIEEGQYCXADPYXCA

SEQ ID NO: 144 - protein, 17 aa, synthetic construct
  REGION 1..17: note = Synthetic Peptide
  SITE 10: note = X - X represents methyl phenylalanine
  SITE 15: note = X - X represents Nle
  MOD_RES 1: note = PEG3 side chain modification
ACIEEGQYCXADPYXCA

SEQ ID NO: 145 - protein, 17 aa, synthetic construct
  REGION 1..17: note = Synthetic Peptide
  SITE 10: note = X - X represents methyl phenylalanine
  SITE 15: note = X - X represents Nle
  MOD_RES 1: note = PEG3 side chain modification
ACIEEGQYCXADPYXCA

SEQ ID NO: 146 - protein, 17 aa, synthetic construct
  REGION 1..17: note = Synthetic Peptide
  SITE 10: note = X - X represents PheG
  SITE 15: note = X - X represents Nle
  MOD_RES 1: note = PEG3 side chain modification
ACIEEGQYCXADPYXCA

SEQ ID NO: 147 - protein, 17 aa, synthetic construct
  REGION 1..17: note = Synthetic Peptide
  SITE 10: note = X - X represents fluorophenylalanine
  SITE 15: note = X - X represents Nle
  MOD_RES 1: note = PEG3 side chain modification
ACIEEGQYCXADPYXCA

SEQ ID NO: 148 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFGDPYXCA

SEQ ID NO: 149 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFSDPYXCA

SEQ ID NO: 150 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFPDPYXCA

SEQ ID NO: 151 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFANPYXCA

SEQ ID NO: 152 - protein, 17 aa, synthetic construct
  REGION 1..17: note = Synthetic Peptide
  SITE 13: note = X - X represents Pipecolic acid
  SITE 15: note = X - X represents Nle
  MOD_RES 1: note = PEG3 side chain modification
ACIEEGQYCFADXYXCA

SEQ ID NO: 153 - protein, 17 aa, synthetic construct
  REGION 1..17: note = Synthetic Peptide
  SITE 13: note = X - X represents Methyl alanine
  SITE 15: note = X - X represents Nle
  MOD_RES 1: note = PEG3 side chain modification
ACIEEGQYCFADXYXCA

SEQ ID NO: 154 - protein, 17 aa, synthetic construct
  REGION 1..17: note = Synthetic Peptide
  SITE 13: note = X - X represents Sarcosine
  SITE 15: note = X - X represents Nle
  MOD_RES 1: note = PEG3 side chain modification
ACIEEGQYCFADXYXCA

SEQ ID NO: 155 - protein, 17 aa, synthetic construct
  REGION 1..17: note = Synthetic Peptide
  SITE 13: note = X - X represents Aib
  SITE 15: note = X - X represents Nle
  MOD_RES 1: note = PEG3 side chain modification
ACIEEGQYCFADXYXCA

SEQ ID NO: 156 - protein, 17 aa, synthetic construct
  REGION 1..17: note = Synthetic Peptide
  SITE 3: note = X - X represents t-butyl-Alanine
  SITE 15: note = X - X represents Nle
  MOD_RES 1: note = PEG3 side chain modification
ACXEEGQYCFADPYXCA

SEQ ID NO: 157 - protein, 17 aa, synthetic construct
  REGION 1..17: note = Synthetic Peptide
  SITE 3: note = X - X represents homoleucine
  SITE 15: note = X - X represents Nle
  MOD_RES 1: note = PEG3 side chain modification
ACXEEGQYCFADPYXCA

SEQ ID NO: 158 - protein, 17 aa, synthetic construct
  REGION 1..17: note = Synthetic Peptide
  SITE 8: note = X - X represents fluorophenylalanine
  SITE 15: note = X - X represents Nle
  MOD_RES 1: note = PEG3 side chain modification
ACIEEGQXCFADPYXCA

SEQ ID NO: 159 - protein, 17 aa, synthetic construct
  REGION 1..17: note = Synthetic Peptide
  SITE 10: note = X - X represents fluorophenylalanine
  SITE 15: note = X - X represents Nle
  MOD_RES 1: note = PEG3 side chain modification
ACIEEGQYCXADPYXCA

SEQ ID NO: 160 - protein, 17 aa, synthetic construct
  REGION 1..17: note = Synthetic Peptide
  SITE 11: note = X - X represents Trifluoromethyl-Alanine
  SITE 15: note = X - X represents Nle
  MOD_RES 1: note = PEG3 side chain modification
ACIEEGQYCFXDPYXCA

SEQ ID NO: 161 - protein, 17 aa, synthetic construct
  REGION 1..17: note = Synthetic Peptide
  SITE 14: note = X - X represents para-Carboxy-Phenylalanine
  SITE 15: note = X - X represents Nle
  MOD_RES 1: note = PEG3 side chain modification
ACIEEGQYCFADPXXCA

SEQ ID NO: 162 - protein, 17 aa, synthetic construct
  REGION 1..17: note = Synthetic Peptide
  SITE 14: note = X - X represents para-Carbamoyl-Phenylalanine
  SITE 15: note = X - X represents Nle
  MOD_RES 1: note = PEG3 side chain modification
ACIEEGQYCFADPXXCA

SEQ ID NO: 163 - protein, 17 aa, synthetic construct
  REGION 1..17: note = Synthetic Peptide
  SITE 15: note = X - X represents Nle
  MOD_RES 1: note = PEG3 side chain modification
ACIEEGQYCFADPQXCA

SEQ ID NO: 164 - protein, 17 aa, synthetic construct
  REGION 1..17: note = Synthetic Peptide
  SITE 14: note = X - X represents 2-methyl phenylalanine
  SITE 15: note = X - X represents Nle
  MOD_RES 1: note = PEG3 side chain modification
ACIEEGQYCFADPXXCA

SEQ ID NO: 165 - protein, 17 aa, synthetic construct
  REGION 1..17: note = Synthetic Peptide
  SITE 14: note = X - X represents 3-methyl phenylalanine
  SITE 15: note = X - X represents Nle
  MOD_RES 1: note = PEG3 side chain modification
ACIEEGQYCFADPXXCA

SEQ ID NO: 166 - protein, 17 aa, synthetic construct
  REGION 1..17: note = Synthetic Peptide
  SITE 14: note = X - X represents 4-methyl phenylalanine
  SITE 15: note = X - X represents Nle
  MOD_RES 1: note = PEG3 side chain modification
ACIEEGQYCFADPXXCA

SEQ ID NO: 167 - protein, 17 aa, synthetic construct
  REGION 1..17: note = Synthetic Peptide
  SITE 14: note = X - X represents citrulline
  SITE 15: note = X - X represents Nle
  MOD_RES 1: note = PEG3 side chain modification
ACIEEGQYCFADPXXCA

SEQ ID NO: 168 - protein, 17 aa, synthetic construct
  REGION 1..17: note = Synthetic Peptide
  SITE 14: note = X - X represents 4-fluorophenylalanine
  SITE 15: note = X - X represents Nle
  MOD_RES 1: note = PEG3 side chain modification
ACIEEGQYCFADPXXCA

SEQ ID NO: 169 - protein, 17 aa, synthetic construct
  REGION 1..17: note = Synthetic Peptide
  SITE 15: note = X - X represents t-butyl-Alanine
  MOD_RES 1: note = PEG3 side chain modification
ACIEEGQYCFADPYXCA

SEQ ID NO: 170 - protein, 17 aa, synthetic construct
  REGION 1..17: note = Synthetic Peptide
  SITE 15: note = X - X represents homoleucine
  MOD_RES 1: note = PEG3 side chain modification
ACIEEGQYCFADPYXCA

SEQ ID NO: 171 - protein, 17 aa, synthetic construct
  REGION 1..17: note = Synthetic Peptide
  MOD_RES 1: note = PEG3 side chain modification
ACIEEGQYCFADPYICA

SEQ ID NO: 172 - protein, 17 aa, synthetic construct
  REGION 1..17: note = Synthetic Peptide
  SITE 15: note = X - X represents 3- cyclohexyl alanine
  MOD_RES 1: note = PEG3 side chain modification
ACIEEGQYCFADPYXCA

SEQ ID NO: 173 - protein, 17 aa, synthetic construct
  REGION 1..17: note = Synthetic Peptide
  MOD_RES 1: note = PEG3 side chain modification
ACIEEGQYCFADPYFCA

SEQ ID NO: 174 - protein, 17 aa, synthetic construct
  REGION 1..17: note = Synthetic Peptide
  SITE 15: note = X - X represents 2-methyl phenylalanine
  MOD_RES 1: note = PEG3 side chain modification
ACIEEGQYCFADPYXCA

SEQ ID NO: 175 - protein, 17 aa, synthetic construct
  REGION 1..17: note = Synthetic Peptide
  SITE 15: note = X - X represents 3-methyl phenylalanine
  MOD_RES 1: note = PEG3 side chain modification
ACIEEGQYCFADPYXCA

SEQ ID NO: 176 - protein, 17 aa, synthetic construct
  REGION 1..17: note = Synthetic Peptide
  SITE 15: note = X - X represents 4-methyl phenylalanine
  MOD_RES 1: note = PEG3 side chain modification
ACIEEGQYCFADPYXCA

SEQ ID NO: 177 - protein, 17 aa, synthetic construct
  REGION 1..17: note = Synthetic Peptide
  SITE 2: note = X - X represents penicillamine
  SITE 15: note = X - X represents Nle
  MOD_RES 1: note = PEG3 side chain modification
AXIEEGQYCFADPYXCA

SEQ ID NO: 178 - protein, 17 aa, synthetic construct
  REGION 1..17: note = Synthetic Peptide
  SITE 9: note = X - X represents penicillamine
  SITE 15: note = X - X represents Nle
  MOD_RES 1: note = PEG3 side chain modification
ACIEEGQYXFADPYXCA

SEQ ID NO: 179 - protein, 17 aa, synthetic construct
  REGION 1..17: note = Synthetic Peptide
  MOD_RES 1: note = PEG3 side chain modification
  SITE 15: note = X - X represents Nle
  SITE 16: note = X - X represents penicillamine
ACIEEGQYCFADPYXXA

SEQ ID NO: 180 - protein, 17 aa, synthetic construct
  REGION 1..17: note = Synthetic Peptide
  SITE 2: note = X - X represents homocysteine
  SITE 15: note = X - X represents Nle
  MOD_RES 1: note = PEG3 side chain modification
AXIEEGQYCFADPYXCA

SEQ ID NO: 181 - protein, 17 aa, synthetic construct
  REGION 1..17: note = Synthetic Peptide
  SITE 9: note = X - X represents homocysteine
  SITE 15: note = X - X represents Nle
  MOD_RES 1: note = PEG3 side chain modification
ACIEEGQYXFADPYXCA

SEQ ID NO: 182 - protein, 17 aa, synthetic construct
  REGION 1..17: note = Synthetic Peptide
  SITE 15: note = X - X represents Nle
  MOD_RES 1: note = PEG3 side chain modification
  SITE 16: note = X - X represents homocysteine
  MOD_RES 1: note = PEG3 side chain modification
ACIEEGQYCFADPYXXA

SEQ ID NO: 183 - protein, 17 aa, synthetic construct
  REGION 1..17: note = Synthetic Peptide
  SITE 10: note = X - X represents 3-fluorophenylalanine
  SITE 15: note = X - X represents Nle
  MOD_RES 1: note = PEG3 side chain modification
ACIEEGQYCXADPYXCA

SEQ ID NO: 184 - protein, 17 aa, synthetic construct
  REGION 1..17: note = Synthetic Peptide
  SITE 4: note = X - X represents tetrazole alanine
  SITE 15: note = X - X represents Nle
  MOD_RES 1: note = PEG3 side chain modification
ACIXEGQYCFADPYXCA

SEQ ID NO: 185 - protein, 17 aa, synthetic construct
  REGION 1..17: note = Synthetic Peptide
  SITE 12: note = X - X represents tetrazole alanine
  SITE 15: note = X - X represents Nle
  MOD_RES 1: note = PEG3 side chain modification
ACIEEGQYCFAXPYXCA

SEQ ID NO: 186 - protein, 17 aa, synthetic construct
  REGION 1..17: note = Synthetic Peptide
  SITE 14: note = X - X represents homoglutamine
  SITE 15: note = X - X represents Nle
  MOD_RES 1: note = PEG3 side chain modification
ACIEEGQYCFADPXXCA

SEQ ID NO: 187 - protein, 17 aa, synthetic construct
  REGION 1..17: note = Synthetic Peptide
  SITE 3: note = X - X represents Aminoheptanoic acid
  SITE 15: note = X - X represents Nle
  MOD_RES 1: note = PEG3 side chain modification
ACXEEGQYCFADPYXCA

SEQ ID NO: 188 - protein, 17 aa, synthetic construct
  REGION 1..17: note = Synthetic Peptide
  SITE 15: note = X - X represents Aminoheptanoic acid
  MOD_RES 1: note = PEG3 side chain modification
ACIEEGQYCFADPYXCA

SEQ ID NO: 189 - protein, 17 aa, synthetic construct
  REGION 1..17: note = Synthetic Peptide
ACIEEGQYCFADPYMCA

SEQ ID NO: 190 - protein, 19 aa, synthetic construct
  REGION 1..19: note = Synthetic Peptide
HEHCIEEGQYCYADPYMCA

SEQ ID NO: 191 - protein, 17 aa, synthetic construct
  REGION 1..17: note = Synthetic Peptide
ACIEEGQYCFADPYLCA

SEQ ID NO: 192 - protein, 17 aa, synthetic construct
  REGION 1..17: note = Synthetic Peptide
ACLPPGPYCFPDPYFCA

SEQ ID NO: 193 - protein, 15 aa, synthetic construct
  REGION 1..15: note = Synthetic Peptide
  SITE 1: note = Acetylation of the N terminus
CIEEGQYCFADPYMC

SEQ ID NO: 194 - protein, 16 aa, synthetic construct
  REGION 1..16: note = Synthetic Peptide
  SITE 14: note = X - X represents Nle
  SITE 16: note = X - X represents diaminopimelic acid
  SITE 1: note = Acetylation of the N terminus
CIEEGQYCFADPYXCX

SEQ ID NO: 195 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-phenylalanine
ACIPEFQYCFADPYXCA

SEQ ID NO: 196 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-phenylalanine
ACIPEFPYCFADPYXCA

SEQ ID NO: 197 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-phenylalanine
ACIEEFPYCFADPYXCA

SEQ ID NO: 198 - protein, 17 aa, synthetic construct
  REGION 1..17: note = Synthetic Peptide
  SITE 15: note = X - X represents Nle
  MOD_RES 1: note = PEG3 side chain modification
ACIPEGPYCFADPYXCA

SEQ ID NO: 199 - protein, 17 aa, synthetic construct
  REGION 1..17: note = Synthetic Peptide
  SITE 3: note = X - X represents t-butyl-Alanine
  SITE 8: note = X - X represents 4-methyl phenylalanine
  SITE 10: note = X - X represents 4-fluorophenylalanine
  SITE 15: note = X - X represents Nle
  MOD_RES 1: note = PEG3 side chain modification
  SITE 7: note = D-phenylalanine
ACXPEFPXCXADPYXCA

SEQ ID NO: 200 - protein, 17 aa, synthetic construct
  REGION 1..17: note = Synthetic Peptide
  SITE 3: note = X - X represents t-butyl-Alanine
  SITE 8: note = X - X represents 4-methyl phenylalanine
  SITE 10: note = X - X represents 4-fluorophenylalanine
  SITE 15: note = X - X represents Nle
  MOD_RES 1: note = PEG3 side chain modification
  SITE 7: note = D-phenylalanine
ACXPEFQXCXADPYXCA

SEQ ID NO: 201 - protein, 17 aa, synthetic construct
  REGION 1..17: note = Synthetic Peptide
  SITE 3: note = X - X represents t-butyl-Alanine
  SITE 8: note = X - X represents 4-methyl phenylalanine
  SITE 10: note = X - X represents 4-fluorophenylalanine
  SITE 15: note = X - X represents Nle
  MOD_RES 1: note = PEG3 side chain modification
  SITE 7: note = D-phenylalanine
ACXEEFPXCXADPYXCA

SEQ ID NO: 202 - protein, 17 aa, synthetic construct
  REGION 1..17: note = Synthetic Peptide
  SITE 3: note = X - X represents t-butyl-Alanine
  SITE 8: note = X - X represents 4-methyl phenylalanine
  SITE 10: note = X - X represents 4-fluorophenylalanine
  SITE 15: note = X - X represents Nle
  MOD_RES 1: note = PEG3 side chain modification
  SITE 7: note = D-phenylalanine
ACXEEFQXCXADPYXCA

SEQ ID NO: 203 - protein, 17 aa, synthetic construct
  REGION 1..17: note = Synthetic Peptide
  SITE 4: note = X - X represents hydroxyproline
  SITE 15: note = X - X represents Nle
  MOD_RES 1: note = PEG3 side chain modification
ACIXEGQYCFADPYXCA

SEQ ID NO: 204 - protein, 17 aa, synthetic construct
  REGION 1..17: note = Synthetic Peptide
  SITE 15: note = X - X represents Nle
  MOD_RES 1: note = PEG3 side chain modification
  SITE 6: note = D-tryptophan
ACIEEWQYCFADPYXCA

SEQ ID NO: 205 - protein, 17 aa, synthetic construct
  REGION 1..17: note = Synthetic Peptide
  SITE 7: note = X - X represents Azetidine
  SITE 15: note = X - X represents Nle
  MOD_RES 1: note = PEG3 side chain modification
ACIEEGXYCFADPYXCA

SEQ ID NO: 206 - protein, 17 aa, synthetic construct
  REGION 1..17: note = Synthetic Peptide
  SITE 7: note = X - X represents Pipecolic acid
  SITE 15: note = X - X represents Nle
  MOD_RES 1: note = PEG3 side chain modification
ACIEEGXYCFADPYXCA

SEQ ID NO: 207 - protein, 17 aa, synthetic construct
  REGION 1..17: note = Synthetic Peptide
  SITE 8: note = X - X represents 2-naphthylalanine
  SITE 15: note = X - X represents Nle
  MOD_RES 1: note = PEG3 side chain modification
ACIEEGQXCFADPYXCA

SEQ ID NO: 208 - protein, 17 aa, synthetic construct
  REGION 1..17: note = Synthetic Peptide
  SITE 8: note = X - X represents 4-methoxy phenylalanine
  SITE 15: note = X - X represents Nle
  MOD_RES 1: note = PEG3 side chain modification
ACIEEGQXCFADPYXCA

SEQ ID NO: 209 - protein, 17 aa, synthetic construct
  REGION 1..17: note = Synthetic Peptide
  SITE 15: note = X - X represents Nle
  MOD_RES 1: note = PEG3 side chain modification
ACIEEGQYCYADPYXCA

SEQ ID NO: 210 - protein, 17 aa, synthetic construct
  REGION 1..17: note = Synthetic Peptide
  SITE 13: note = X - X represents Azetidine
  SITE 15: note = X - X represents Nle
  MOD_RES 1: note = PEG3 side chain modification
ACIEEGQYCFADXYXCA

SEQ ID NO: 211 - protein, 17 aa, synthetic construct
  REGION 1..17: note = Synthetic Peptide
  SITE 15: note = X - X represents Homoserine(Me)
  MOD_RES 1: note = PEG3 side chain modification
ACIEEGQYCFADPYXCA

SEQ ID NO: 212 - protein, 17 aa, synthetic construct
  REGION 1..17: note = Synthetic Peptide
  SITE 3: note = X - X represents N-Methyl-lsoleucine
  SITE 15: note = X - X represents Nle
  SITE 1: note = Acetylation of the N-terminus
ACXEEGQYCFADPYXCA

SEQ ID NO: 213 - protein, 17 aa, synthetic construct
  REGION 1..17: note = Synthetic Peptide
  SITE 4: note = X - X represents Azetidine
  SITE 15: note = X - X represents Nle
  SITE 1: note = Acetylation of the N-terminus
ACIXEGQYCFADPYXCA

SEQ ID NO: 214 - protein, 17 aa, synthetic construct
  REGION 1..17: note = Synthetic Peptide
  SITE 4: note = X - X represents Pipecolic acid
  SITE 15: note = X - X represents Nle
  SITE 1: note = Acetylation of the N-terminus
ACIXEGQYCFADPYXCA

SEQ ID NO: 215 - protein, 17 aa, synthetic construct
  REGION 1..17: note = Synthetic Peptide
  SITE 4: note = X - X represents N-methyl glutamic acid
  SITE 15: note = X - X represents Nle
  SITE 1: note = Acetylation of the N-terminus
ACIXEGQYCFADPYXCA

SEQ ID NO: 216 - protein, 17 aa, synthetic construct
  REGION 1..17: note = Synthetic Peptide
  SITE 1: note = Acetylation of the N-terminus
  SITE 5: note = X - X represents N-methyl glutamic acid
  SITE 15: note = X - X represents Nle
ACIEXGQYCFADPYXCA

SEQ ID NO: 217 - protein, 17 aa, synthetic construct
  REGION 1..17: note = Synthetic Peptide
  SITE 15: note = X - X represents Nle
  SITE 1: note = Acetylation of the N-terminus
  SITE 6: note = D-aspartic acid
ACIEEDQYCFADPYXCA

SEQ ID NO: 218 - protein, 17 aa, synthetic construct
  REGION 1..17: note = Synthetic Peptide
  SITE 7: note = X - X represents N-methyl alanine
  SITE 15: note = X - X represents Nle
  SITE 1: note = Acetylation of the N-terminus
ACIEEGXYCFADPYXCA

SEQ ID NO: 219 - protein, 17 aa, synthetic construct
  REGION 1..17: note = Synthetic Peptide
  SITE 8: note = X - X represents N-methyl tyrosine
  SITE 15: note = X - X represents Nle
  SITE 1: note = Acetylation of the N-terminus
ACIEEGQXCFADPYXCA

SEQ ID NO: 220 - protein, 17 aa, synthetic construct
  REGION 1..17: note = Synthetic Peptide
  SITE 8: note = X - X represents homophenylalanine
  SITE 15: note = X - X represents Nle
  SITE 1: note = Acetylation of the N-terminus
ACIEEGQXCFADPYXCA

SEQ ID NO: 221 - protein, 17 aa, synthetic construct
  REGION 1..17: note = Synthetic Peptide
  SITE 8: note = X - X represents 2-pyridylalanine
  SITE 15: note = X - X represents Nle
  SITE 1: note = Acetylation of the N-terminus
ACIEEGQXCFADPYXCA

SEQ ID NO: 222 - protein, 17 aa, synthetic construct
  REGION 1..17: note = Synthetic Peptide
  SITE 8: note = X - X represents 3-pyridylalanine
  SITE 15: note = X - X represents Nle
  SITE 1: note = Acetylation of the N-terminus
ACIEEGQXCFADPYXCA

SEQ ID NO: 223 - protein, 17 aa, synthetic construct
  REGION 1..17: note = Synthetic Peptide
  SITE 8: note = X - X represents 4,4-Biphenylalanine
  SITE 15: note = X - X represents Nle
  SITE 1: note = Acetylation of the N-terminus
ACIEEGQXCFADPYXCA

SEQ ID NO: 224 - protein, 17 aa, synthetic construct
  REGION 1..17: note = Synthetic Peptide
  SITE 10: note = X - X represents homophenylalanine
  SITE 15: note = X - X represents Nle
  SITE 1: note = Acetylation of the N-terminus
ACIEEGQYCXADPYXCA

SEQ ID NO: 225 - protein, 17 aa, synthetic construct
  REGION 1..17: note = Synthetic Peptide
  SITE 10: note = X - X represents 2-pyridylalanine
  SITE 15: note = X - X represents Nle
  SITE 1: note = Acetylation of the N-terminus
ACIEEGQYCXADPYXCA

SEQ ID NO: 226 - protein, 17 aa, synthetic construct
  REGION 1..17: note = Synthetic Peptide
  SITE 10: note = X - X represents 3-pyridylalanine
  SITE 15: note = X - X represents Nle
  SITE 1: note = Acetylation of the N-terminus
ACIEEGQYCXADPYXCA

SEQ ID NO: 227 - protein, 17 aa, synthetic construct
  REGION 1..17: note = Synthetic Peptide
  SITE 10: note = X - X represents 4-pyridylalanine
  SITE 15: note = X - X represents Nle
  SITE 1: note = Acetylation of the N-terminus
ACIEEGQYCXADPYXCA

SEQ ID NO: 228 - protein, 17 aa, synthetic construct
  REGION 1..17: note = Synthetic Peptide
  SITE 10: note = X - X represents 4,4-Biphenylalanine
  SITE 15: note = X - X represents Nle
  SITE 1: note = Acetylation of the N-terminus
ACIEEGQYCXADPYXCA

SEQ ID NO: 229 - protein, 17 aa, synthetic construct
  REGION 1..17: note = Synthetic Peptide
  SITE 10: note = X - X represents 1-naphthylalanine
  SITE 15: note = X - X represents Nle
  SITE 1: note = Acetylation of the N-terminus
ACIEEGQYCXADPYXCA

SEQ ID NO: 230 - protein, 17 aa, synthetic construct
  REGION 1..17: note = Synthetic Peptide
  SITE 10: note = X - X represents 4-t-butyl phenylalanine
  SITE 15: note = X - X represents Nle
  SITE 1: note = Acetylation of the N-terminus
ACIEEGQYCXADPYXCA

SEQ ID NO: 231 - protein, 17 aa, synthetic construct
  REGION 1..17: note = Synthetic Peptide
  SITE 11: note = X - X represents N-methyl alanine
  SITE 15: note = X - X represents Nle
  SITE 1: note = Acetylation of the N-terminus
ACIEEGQYCFXDPYXCA

SEQ ID NO: 232 - protein, 17 aa, synthetic construct
  REGION 1..17: note = Synthetic Peptide
  SITE 7: note = X - X represents 5,5-Dimethyl-L-Proline
  SITE 15: note = X - X represents Nle
  SITE 1: note = Acetylation of the N-terminus
ACIEEGXYCFADPYXCA

SEQ ID NO: 233 - protein, 17 aa, synthetic construct
  REGION 1..17: note = Synthetic Peptide
  SITE 13: note = X - X represents hydroxyproline
  SITE 15: note = X - X represents Nle
  SITE 1: note = Acetylation of the N-terminus
ACIEEGQYCFADXYXCA

SEQ ID NO: 234 - protein, 17 aa, synthetic construct
  REGION 1..17: note = Synthetic Peptide
  SITE 7: note = X - X represents octahydroindolecarboxylic acid
  SITE 15: note = X - X represents Nle
  SITE 1: note = Acetylation of the N-terminus
ACIEEGXYCFADPYXCA

SEQ ID NO: 235 - protein, 17 aa, synthetic construct
  REGION 1..17: note = Synthetic Peptide
  SITE 13: note = X - X represents octahydroindolecarboxylic acid
  SITE 15: note = X - X represents Nle
  SITE 1: note = Acetylation of the N-terminus
ACIEEGQYCFADXYXCA

SEQ ID NO: 236 - protein, 17 aa, synthetic construct
  REGION 1..17: note = Synthetic Peptide
  SITE 4: note = X - X represents octahydroindolecarboxylic acid
  SITE 15: note = X - X represents Nle
  SITE 1: note = Acetylation of the N-terminus
ACIXEGQYCFADPYXCA

SEQ ID NO: 237 - protein, 17 aa, synthetic construct
  REGION 1..17: note = Synthetic Peptide
  SITE 13: note = X - X represents oxazolidine-4-carboxylic acid
  SITE 15: note = X - X represents Nle
  SITE 1: note = Acetylation of the N-terminus
ACIEEGQYCFADXYXCA

SEQ ID NO: 238 - protein, 17 aa, synthetic construct
  REGION 1..17: note = Synthetic Peptide
  SITE 4: note = X - X represents oxazolidine-4-carboxylic acid
  SITE 15: note = X - X represents Nle
  SITE 1: note = Acetylation of the N-terminus
ACIXEGQYCFADPYXCA

SEQ ID NO: 239 - protein, 17 aa, synthetic construct
  REGION 1..17: note = Synthetic Peptide
  SITE 7: note = X - X represents oxazolidine-4-carboxylic acid
  SITE 15: note = X - X represents Nle
  SITE 1: note = Acetylation of the N-terminus
ACIEEGXYCFADPYXCA

SEQ ID NO: 240 - protein, 17 aa, synthetic construct
  REGION 1..17: note = Synthetic Peptide
  SITE 15: note = X - X represents Nle
  SITE 1: note = Acetylation of the N-terminus
ACIPEGPYCFADPYXCA

SEQ ID NO: 241 - protein, 17 aa, synthetic construct
  REGION 1..17: note = Synthetic Peptide
  SITE 7: note = X - X represents hydroxyproline
  SITE 15: note = X - X represents Nle
  SITE 1: note = Acetylation of the N-terminus
ACIEEGXYCFADPYXCA

SEQ ID NO: 242 - protein, 17 aa, synthetic construct
  REGION 1..17: note = Synthetic Peptide
  SITE 15: note = X - X represents Nle
  SITE 1: note = Acetylation of the N-terminus
  SITE 6: note = D-alanine
ACIPEAPYCFADPYXCA

SEQ ID NO: 243 - protein, 17 aa, synthetic construct
  REGION 1..17: note = Synthetic Peptide
  SITE 3: note = X - X represents t-butyl-Alanine
  SITE 15: note = X - X represents Nle
  SITE 1: note = Acetylation of the N-terminus
  SITE 6: note = D-alanine
ACXPEAPYCFADPYXCA

SEQ ID NO: 244 - protein, 17 aa, synthetic construct
  REGION 1..17: note = Synthetic Peptide
  SITE 15: note = X - X represents Nle
  SITE 11: note = D-alanine
ACIEEGQYCFADPYXCA

SEQ ID NO: 245 - protein, 17 aa, synthetic construct
  REGION 1..17: note = Synthetic Peptide
  SITE 3: note = X - X represents t-butyl-Alanine
  SITE 15: note = X - X represents Nle
  SITE 1: note = Acetylation of the N-terminus
ACXEEGQYCFADPYXCA

SEQ ID NO: 246 - protein, 17 aa, synthetic construct
  REGION 1..17: note = Synthetic Peptide
  SITE 3: note = X - X represents L-Cyclohexyl glycine
  SITE 15: note = X - X represents Nle
  SITE 1: note = Acetylation of the N-terminus
ACXEEGQYCFADPYXCA

SEQ ID NO: 247 - protein, 17 aa, synthetic construct
  REGION 1..17: note = Synthetic Peptide
  SITE 3: note = X - X represents Aminocyclopentanecarboxylic acid
  SITE 15: note = X - X represents Nle
  SITE 1: note = Acetylation of the N-terminus
ACXEEGQYCFADPYXCA

SEQ ID NO: 248 - protein, 17 aa, synthetic construct
  REGION 1..17: note = Synthetic Peptide
  SITE 15: note = X - X represents Nle
  SITE 1: note = Acetylation of the N-terminus
ACIPEGQYCFADPYXCA

SEQ ID NO: 249 - protein, 17 aa, synthetic construct
  REGION 1..17: note = Synthetic Peptide
  SITE 15: note = X - X represents Nle
  SITE 1: note = Acetylation of the N-terminus
ACIQEGQYCFADPYXCA

SEQ ID NO: 250 - protein, 17 aa, synthetic construct
  REGION 1..17: note = Synthetic Peptide
  SITE 15: note = X - X represents Nle
  SITE 1: note = Acetylation of the N-terminus
ACIEPGQYCFADPYXCA

SEQ ID NO: 251 - protein, 17 aa, synthetic construct
  REGION 1..17: note = Synthetic Peptide
  SITE 15: note = X - X represents Nle
  SITE 1: note = Acetylation of the N-terminus
ACIEQGQYCFADPYXCA

SEQ ID NO: 252 - protein, 17 aa, synthetic construct
  REGION 1..17: note = Synthetic Peptide
  SITE 15: note = X - X represents Nle
  SITE 1: note = Acetylation of the N-terminus
  SITE 1: note = D-phenylalanine
ACIEEFQYCFADPYXCA

SEQ ID NO: 253 - protein, 17 aa, synthetic construct
  REGION 1..17: note = Synthetic Peptide
  SITE 15: note = X - X represents Nle
  SITE 1: note = Acetylation of the N-terminus
  SITE 1: note = D-alanine
ACIEEAQYCFADPYXCA

SEQ ID NO: 254 - protein, 17 aa, synthetic construct
  REGION 1..17: note = Synthetic Peptide
  SITE 6: note = X - X represents Aminocyclopentanecarboxylic acid
  SITE 15: note = X - X represents Nle
  SITE 1: note = Acetylation of the N-terminus
ACIEEXQYCFADPYXCA

SEQ ID NO: 255 - protein, 17 aa, synthetic construct
  REGION 1..17: note = Synthetic Peptide
  SITE 15: note = X - X represents Nle
  SITE 1: note = Acetylation of the N-terminus
ACIEEGAYCFADPYXCA

SEQ ID NO: 256 - protein, 17 aa, synthetic construct
  REGION 1..17: note = Synthetic Peptide
  SITE 7: note = X - X represents Aib
  SITE 15: note = X - X represents Nle
  SITE 1: note = Acetylation of the N-terminus
ACIEEGXYCFADPYXCA

SEQ ID NO: 257 - protein, 17 aa, synthetic construct
  REGION 1..17: note = Synthetic Peptide
  SITE 7: note = X - X represents Aminocyclopentanecarboxylic acid
  SITE 15: note = X - X represents Nle
  SITE 1: note = Acetylation of the N-terminus
ACIEEGXYCFADPYXCA

SEQ ID NO: 258 - protein, 17 aa, synthetic construct
  REGION 1..17: note = Synthetic Peptide
  SITE 8: note = X - X represents 4-methyl phenylalanine
  SITE 15: note = X - X represents Nle
  SITE 1: note = Acetylation of the N-terminus
ACIEEGQXCFADPYXCA

SEQ ID NO: 259 - protein, 17 aa, synthetic construct
  REGION 1..17: note = Synthetic Peptide
  SITE 8: note = X - X represnets 1-naphthylalanine
  SITE 15: note = X - X represnets Nle
  SITE 1: note = Acetylation of the N-terminus
ACIEEGQXCFADPYXCA

SEQ ID NO: 260 - protein, 17 aa, synthetic construct
  REGION 1..17: note = Synthetic Peptide
  SITE 10: note = X - X represents 2-naphthylalanine
  SITE 15: note = X - X represents Nle
  SITE 1: note = Acetylation of the N-terminus
ACIEEGQYCXADPYXCA

SEQ ID NO: 261 - protein, 17 aa, synthetic construct
  REGION 1..17: note = Synthetic Peptide
  SITE 10: note = X - X represents nitro phenylalanine
  SITE 15: note = X - X represents Nle
  SITE 1: note = Acetylation of the N-terminus
ACIEEGQYCXADPYXCA

SEQ ID NO: 262 - protein, 17 aa, synthetic construct
  REGION 1..17: note = Synthetic Peptide
  SITE 10: note = X - X represents 4-bromo phenylalanine
  SITE 15: note = X - X represents Nle
  SITE 1: note = Acetylation of the N-terminus
ACIEEGQYCXADPYXCA

SEQ ID NO: 263 - protein, 17 aa, synthetic construct
  REGION 1..17: note = Synthetic Peptide
  SITE 11: note = X - X represents Abu
  SITE 15: note = X - X represents Nle
  SITE 1: note = Acetylation of the N-terminus
ACIEEGQYCFXDPYXCA

SEQ ID NO: 264 - protein, 17 aa, synthetic construct
  REGION 1..17: note = Synthetic Peptide
  SITE 15: note = X - X represents Aminoheptanoic acid
  SITE 1: note = Acetylation of the N-terminus
ACIEEGQYCFADPYXCA

SEQ ID NO: 265 - protein, 17 aa, synthetic construct
  REGION 1..17: note = Synthetic Peptide
  SITE 1: note = Acetylation of the N-terminus
ACIEEGQYCFADPYMCA

SEQ ID NO: 266 - protein, 15 aa, synthetic construct
  REGION 1..15: note = Synthetic Peptide
  SITE 2: note = X - X represents Isoleucine, t-butyl-Alanine or L-Cyclohexyl glycine
  SITE 3: note = X - X represents Glutamic acid, Proline, Aspartic acid, Lysine, Aad, hydroxyproline or oxazolidine-4-carboxylic acid
  SITE 4: note = X - X represents Glutamic acid, Lysine or Aad
  SITE 5: note = X - X represents Glysine, D-Lysine, D-Alanine, L-Alanine, D-Phenylalanine, D-Glutamic acid, D-Glutamine, D-Leucine, D-Serine or D-Tryptophan
  SITE 6: note = X - X represents Glutamine, Lysine, Alanine, Proline, 5,5-Dimethyl-L-Proline, octahydroindolecarboxylic acid, oxazolidine-4-carboxylic acid, hydroxyproline, Aib or Aminocyclopentanecarboxylic acid
  SITE 7: note = X - X represents Tyrosine, Phenylalanine, 3-methyl phenylalanine , 4-methyl phenylalanine , 4-fluorophenylalanine, 2-naphthylalanine, 4-methoxy phenylalanine or 4,4-Biphenylalanine
  SITE 9: note = X - X represents Phenylalanine, Lysine, 4-methyl phenylalanine , 2-fluorophenylalanine , 4-fluorophenylalanine , 4-pyridylalanine, 4,4-Biphenylalanine , 4-t-butyl-Alanine, nitro phenylalanine or 4-bromo phenylalanine
  SITE 10: note = X - X represents Alanine or Lysine
  SITE 12: note = X - X represents Proline or Lysine
  SITE 13: note = X - X represents Tyrosine or Lysine
  SITE 14: note = X - X represents Methionine, Lysine, Nle, homoleucine or Aminoheptanoic acid
CXXXXXXCXXDXXXC

SEQ ID NO: 267 - protein, 15 aa, synthetic construct
  REGION 1..15: note = Synthetic Peptide
  SITE 2: note = X - X represents Isoleucine or t-butyl-Alanine
  SITE 3: note = X - X represents Lysine, Glutamic acid or Proline
  SITE 4: note = X - X represents Glutamic acid or D-Lysine
  SITE 5: note = X - X represents Glycine, D-Lysine, D-Phenylalanine or D-Alanine
  SITE 6: note = X - X represents Glutamine, Lysine or Proline
  SITE 7: note = X - X represents Tyrosine or 4-methyl phenylalanine
  SITE 9: note = X - X represents Phenylalanine or 4-fluorophenylalanine
  SITE 14: note = X - X represents Methionine or Nle
CXXXXXXCXADPYXC

SEQ ID NO: 268 - protein, 15 aa, synthetic construct
  MOD_RES 4: note = PEG12 side chain modification
CIKEGQYCFADPYMC

SEQ ID NO: 269 - protein, 15 aa, synthetic construct
  MOD_RES 5: note = PEG12 side chain modification
CIEKGQYCFADPYMC

SEQ ID NO: 270 - protein, 15 aa, synthetic construct
  MOD_RES 7: note = PEG12 side chain modification
CIEEGKYCFADPYMC

SEQ ID NO: 271 - protein, 15 aa, synthetic construct
  MOD_RES 10: note = PEG12 side chain modification
CIEEGQYCKADPYMC

SEQ ID NO: 272 - protein, 15 aa, synthetic construct
  MOD_RES 11: note = PEG12 side chain modification
CIEEGQYCFKDPYMC

SEQ ID NO: 273 - protein, 15 aa, synthetic construct
  MOD_RES 12: note = PEG12 side chain modification
CIEEGQYCFAKPYMC

SEQ ID NO: 274 - protein, 15 aa, synthetic construct
  MOD_RES 13: note = PEG12 side chain modification
CIEEGQYCFADKYMC

SEQ ID NO: 275 - protein, 15 aa, synthetic construct
  MOD_RES 14: note = PEG12 side chain modification
CIEEGQYCFADPKMC

SEQ ID NO: 276 - protein, 15 aa, synthetic construct
  MOD_RES 15: note = PEG12 side chain modification
CIEEGQYCFADPYKC

SEQ ID NO: 277 - protein, 15 aa, synthetic construct
  SITE 5: note = D-lysine
  MOD_RES 5: note = PEG12Fl side chain modification
  SITE 14: note = X represents Nle
CIEEKQYCFADPYXC

SEQ ID NO: 278 - protein, 15 aa, synthetic construct
CIEEGAYCFADPYXC

SEQ ID NO: 279 - protein, 15 aa, synthetic construct
CIEEAQYCFADPYXC

SEQ ID NO: 280 - protein, 15 aa, synthetic construct
  SITE 2: note = X represents L-Cyclohexyl glycine
  SITE 14: note = X represents Nle
CXEEGQYCFADPYXC

SEQ ID NO: 281 - protein, 15 aa, synthetic construct
  SITE 14: note = X represents Nle
CIPEGQYCFADPYXC

SEQ ID NO: 282 - protein, 15 aa, synthetic construct
  SITE 14: note = X represents Nle
CIDEGQYCFADPYXC

SEQ ID NO: 283 - protein, 15 aa, synthetic construct
  SITE 3: note = X represents Aad
  SITE 14: note = X represents Nle
CIXEGQYCFADPYXC

SEQ ID NO: 284 - protein, 15 aa, synthetic construct
  SITE 4: note = X represents Aad
  SITE 14: note = X represents Nle
CIEXGQYCFADPYXC

SEQ ID NO: 285 - protein, 15 aa, synthetic construct
  SITE 5: note = D-lysine
  SITE 14: note = X represents Nle
CIEEKQYCFADPYXC

SEQ ID NO: 286 - protein, 15 aa, synthetic construct
  SITE 5: note = D-phenylalanine
  SITE 14: note = X represents Nle
CIEEFQYCFADPYXC

SEQ ID NO: 287 - protein, 15 aa, synthetic construct
  SITE 5: note = D-glutamic acid
  SITE 14: note = X represents Nle
CIEEEQYCFADPYXC

SEQ ID NO: 288 - protein, 15 aa, synthetic construct
  SITE 5: note = D-glutamine
  SITE 14: note = X represents Nle
CIEEQQYCFADPYXC

SEQ ID NO: 289 - protein, 15 aa, synthetic construct
  SITE 5: note = D-leucine
  SITE 14: note = X represents Nle
CIEELQYCFADPYXC

SEQ ID NO: 290 - protein, 15 aa, synthetic construct
  SITE 5: note = D-serine
  SITE 14: note = X represents Nle
CIEESQYCFADPYXC

SEQ ID NO: 291 - protein, 15 aa, synthetic construct
  SITE 14: note = X represents Nle
CIEEGPYCFADPYXC

SEQ ID NO: 292 - protein, 15 aa, synthetic construct
  SITE 14: note = X represents Nle
CIEEGQFCFADPYXC

SEQ ID NO: 293 - protein, 15 aa, synthetic construct
  SITE 7: note = X represents 3-methyl phenylalanine
  SITE 14: note = X represents Nle
CIEEGQXCFADPYXC

SEQ ID NO: 294 - protein, 15 aa, synthetic construct
  SITE 7: note = X represents 4-methyl phenylalanine
  SITE 14: note = X represents Nle
CIEEGQXCFADPYXC

SEQ ID NO: 295 - protein, 15 aa, synthetic construct
  SITE 7: note = X represents 4-fluoro phenylalanine
  SITE 14: note = X represents Nle
CIEEGQXCFADPYXC

SEQ ID NO: 296 - protein, 15 aa, synthetic construct
  SITE 9: note = X represents 4-Methyl phenylalanine
  SITE 14: note = X represents Nle
CIEEGQYCXADPYXC

SEQ ID NO: 297 - protein, 15 aa, synthetic construct
  SITE 9: note = X represents 4-fluorophenylalanine
  SITE 14: note = X represents Nle
CIEEGQYCXADPYXC

SEQ ID NO: 298 - protein, 15 aa, synthetic construct
  SITE 2: note = X represents t-Butyl Alanine
  SITE 14: note = X represents Nle
CXEEGQYCFADPYXC

SEQ ID NO: 299 - protein, 15 aa, synthetic construct
  SITE 9: note = X represents 2-fluoro phenylalanine
  SITE 14: note = X represents Nle
CIEEGQYCXADPYXC

SEQ ID NO: 300 - protein, 15 aa, synthetic construct
  SITE 14: note = X represents homoleucine
CIEEGQYCFADPYXC

SEQ ID NO: 301 - protein, 15 aa, synthetic construct
  SITE 14: note = X represents Aminoheptanoic acid
CIEEGQYCFADPYXC

SEQ ID NO: 302 - protein, 15 aa, synthetic construct
  SITE 5: note = D-phenylalanine
  SITE 14: note = X represents Nle
CIPEFQYCFADPYXC

SEQ ID NO: 303 - protein, 15 aa, synthetic construct
  SITE 5: note = D-phenylalanine
  SITE 14: note = X represents Nle
CIPEFPYCFADPYXC

SEQ ID NO: 304 - protein, 15 aa, synthetic construct
  SITE 5: note = D-phenylalanine
  SITE 14: note = X represents Nle
CIEEFPYCFADPYXC

SEQ ID NO: 305 - protein, 15 aa, synthetic construct
  SITE 14: note = X represents Nle
CIPEGPYCFADPYXC

SEQ ID NO: 306 - protein, 15 aa, synthetic construct
  SITE 3: note = X represents t-Butyl Alanine
  SITE 5: note = D-phenylalanine
  SITE 7: note = X represents 4-methyl phenylalanine
  SITE 9: note = X represents 4-fluorophenylalanine
  SITE 14: note = X represents Nle
CXPEFPXCXADPYXC

SEQ ID NO: 307 - protein, 15 aa, synthetic construct
  SITE 3: note = X represents t-Butyl Alanine
  SITE 5: note = D-phenylalanine
  SITE 7: note = X represents 4-methyl phenylalanine
  SITE 9: note = X represents 4-fluorophenylalanine
  SITE 14: note = X represents Nle
CXPEFQXCXADPYXC

SEQ ID NO: 308 - protein, 15 aa, synthetic construct
  SITE 3: note = X represents t-Butyl Alanine
  SITE 5: note = D-phenylalanine
  SITE 7: note = X represents 4-methyl phenylalanine
  SITE 9: note = X represents 4-fluorophenylalanine
  SITE 14: note = X represents Nle
CXEEFPXCXADPYXC

SEQ ID NO: 309 - protein, 15 aa, synthetic construct
  SITE 3: note = X represents hydroxyproline
  SITE 14: note = X represents Nle
CIXEGQYCFADPYXC

SEQ ID NO: 310 - protein, 15 aa, synthetic construct
  SITE 5: note = D-Tryptophan
  SITE 14: note = X represents Nle
CIEEWQYCFADPYXC

SEQ ID NO: 311 - protein, 15 aa, synthetic construct
  SITE 7: note = X represents 2-naphthylalanine
  SITE 14: note = X represents Nle
CIEEGQXCFADPYXC

SEQ ID NO: 312 - protein, 15 aa, synthetic construct
  SITE 7: note = X represents methoxy phenylalanine
  SITE 14: note = X represents Nle
CIEEGQXCFADPYXC

SEQ ID NO: 313 - protein, 15 aa, synthetic construct
  SITE 7: note = X represents 4,4-Biphenylalanine
  SITE 14: note = X represents Nle
CIEEGQXCFADPYXC

SEQ ID NO: 314 - protein, 15 aa, synthetic construct
  SITE 9: note = X represents 4-pyridylalanine
  SITE 14: note = X represents Nle
CIEEGQYCXADPYXC

SEQ ID NO: 315 - protein, 15 aa, synthetic construct
  SITE 9: note = X represents 4,4-Biphenylalanine
  SITE 14: note = X represents Nle
CIEEGQYCXADPYXC

SEQ ID NO: 316 - protein, 15 aa, synthetic construct
  SITE 9: note = X represents 4-t-Butyl Phenylalanine
  SITE 14: note = X represents Nle
CIEEGQYCXADPYXC

SEQ ID NO: 317 - protein, 15 aa, synthetic construct
  SITE 6: note = X represents 5,5-Dimethyl-L-Proline
  SITE 14: note = X represents Nle
CIEEGXYCFADPYXC

SEQ ID NO: 318 - protein, 15 aa, synthetic construct
  SITE 6: note = X represents octahydroindolecarboxylic acid
  SITE 14: note = X represents Nle
CIEEGXYCFADPYXC

SEQ ID NO: 319 - protein, 15 aa, synthetic construct
  SITE 2: note = X represents oxazolidine-4-carboxylic acid
  SITE 14: note = X represents Nle
CIXEGQYCFADPYXC

SEQ ID NO: 320 - protein, 15 aa, synthetic construct
  SITE 6: note = X represents oxazolidine-4-carboxylic acid
  SITE 14: note = X represents Nle
CIEEGXYCFADPYXC

SEQ ID NO: 321 - protein, 15 aa, synthetic construct
  SITE 14: note = X represents Nle
CIPEGPYCFADPYXC

SEQ ID NO: 322 - protein, 15 aa, synthetic construct
  SITE 6: note = X represents hydroxyproline
  SITE 14: note = X represents Nle
CIEEGXYCFADPYXC

SEQ ID NO: 323 - protein, 15 aa, synthetic construct
  SITE 5: note = D-alanine
  SITE 14: note = X represents Nle
CIPEAPYCFADPYXC

SEQ ID NO: 324 - protein, 15 aa, synthetic construct
  SITE 2: note = X represents t-Butyl Alanine
  SITE 14: note = X represents Nle
CXEEGQYCFADPYXC

SEQ ID NO: 325 - protein, 15 aa, synthetic construct
  SITE 14: note = X represents Nle
CIPEGQYCFADPYXC

SEQ ID NO: 326 - protein, 15 aa, synthetic construct
  SITE 5: note = D-phenylalanine
  SITE 14: note = X represents Nle
CIEEFQYCFADPYXC

SEQ ID NO: 327 - protein, 15 aa, synthetic construct
  SITE 6: note = X represents aminoisobutyric acid
  SITE 14: note = X represents Nle
CIEEGXYCFADPYXC

SEQ ID NO: 328 - protein, 15 aa, synthetic construct
  SITE 6: note = X represents Aminocyclopentanecarboxylic acid
  SITE 14: note = X represents Nle
CIEEGXYCFADPYXC

SEQ ID NO: 329 - protein, 15 aa, synthetic construct
  SITE 9: note = X represents nitro phenylalanine
  SITE 14: note = X represents Nle
CIEEGQYCXADPYXC

SEQ ID NO: 330 - protein, 15 aa, synthetic construct
  SITE 9: note = X represents 4-bromo phenylalanine
  SITE 14: note = X represents Nle
CIEEGQYCXADPYXC

SEQ ID NO: 331 - protein, 15 aa, synthetic construct
CIEEGQYCFADPYMC

SEQ ID NO: 332 - protein, 15 aa, synthetic construct
CIEEGQYCFADPYXC

SEQ ID NO: 333 - protein, 15 aa, synthetic construct
  SITE 14: note = X represents Nle
CIEEGQYCFADPYXC

SEQ ID NO: 334 - protein, 15 aa, synthetic construct
  SITE 2: note = X represents t-Butyl Alanine
  SITE 5: note = D-alanine
  SITE 14: note = X represents Nle
CXPEAPYCFADPYXC

SEQ ID NO: 335 - protein, 15 aa, synthetic construct
  MOD_RES 4: note = PEG12 side chain modification
CIKEGQYCFADPYMC

SEQ ID NO: 336 - protein, 15 aa, synthetic construct
  MOD_RES 4: note = PEG12 side chain modification
CIEKGQYCFADPYMC

SEQ ID NO: 337 - protein, 15 aa, synthetic construct
  MOD_RES 6: note = PEG12 side chain modification
CIEEGKYCFADPYMC

SEQ ID NO: 338 - protein, 15 aa, synthetic construct
  SITE 4: note = D-lysine
  MOD_RES 4: note = PEG12Fl side chain modification
  SITE 14: note = X - X represents Nle
CIEEKQYCFADPYXC

SEQ ID NO: 339 - protein, 15 aa, synthetic construct
  SITE 14: note = X - X represents Nle
CIPEGQYCFADPYXC

SEQ ID NO: 340 - protein, 15 aa, synthetic construct
  SITE 5: note = D-phenylalanine
  SITE 14: note = X - X represents Nle
CIEEFQYCFADPYXC

SEQ ID NO: 341 - protein, 15 aa, synthetic construct
  SITE 14: note = X - X represents Nle
CIEEGPYCFADPYXC

SEQ ID NO: 342 - protein, 15 aa, synthetic construct
  SITE 2: note = X - X represents t-Butyl Alanine
  SITE 5: note = D-Phenylalanine
  SITE 7: note = X - X represents 4-methyl phenylalanine
  SITE 9: note = X - X represents 4-fluorophenylalanine
  SITE 14: note = X - X represents Nle
CXPEFPXCXADPYXC

SEQ ID NO: 343 - protein, 15 aa, synthetic construct
CIEEGQYCFADPYMC

SEQ ID NO: 344 - protein, 15 aa, synthetic construct
  SITE 14: note = X - X represents Nle
CIEEGQYCFADPYXC

SEQ ID NO: 345 - protein, 15 aa, synthetic construct
  SITE 14: note = X - X represents Nle
CIEEGQYCFADPYXC

SEQ ID NO: 346 - protein, 15 aa, synthetic construct
  SITE 2: note = X - X represents t-Butyl Alanine
  SITE 5: note = D-alanine
  SITE 14: note = X - X represents Nle
CXPEAPYCFADPYXC